SEQUENCE LISTING (127 sequences)

SEQ ID NO: 1 - DNA, 300 bp, Dirofilaria immitis
  source 1..300: mol_type = genomic DNA
aacataaacatattgaactgaatcctgcaaacagttctcttataacgtgaaccataactaaatttagagaaaatatgaaaaagaaaaataagttgcttttgctcgtgcaccaactctaatacccaggaaatcaagaagtgataatgagtaatgtcatcattagattcagtaattggtgacactatcaatattattattattatacttaaaaatacgacgaccacttatcgtaacttaaagcatgcataatacgactgtcatcatattacatttcttcaagttcgtattggacaagtgatt

SEQ ID NO: 2 - DNA, 300 bp, Dirofilaria immitis
  source 1..300: mol_type = genomic DNA
gacaagcgttgacgggagagacgatataataataaagaaggcattgggtatcagaaggcacaatccaattataaatgccaaggcaaaatgaataaaatttatgctgacgatttgatcaattacgaagaatttccgatcggctcgaatctttgtttgtatgtgcactactgttaacttaatctttgttttatatacttttgcgtgtcatatataatatattcatgtcaactgatacgttatgatgtttttttgtaaattaagttgatcggaaacctgaagtctatttcaaatttaagaaat

SEQ ID NO: 3 - DNA, 300 bp, Dirofilaria immitis
  source 1..300: mol_type = genomic DNA
ttttaggaaaatggtgactgtagagagatattatcggaacgacaaggtccacttcgaacgggtcttttattgtcgacggattgtgaaccaagttttggcattcataatgacaggtagctatttttccatcatcccatttttgtattagtgcaagcaagtcatgagtcgaaagaaaatctcaaaagaaaaaaatgaaatttcaggttcaaaggactgcgtccattattcgcactggttgatgagaacgtacagattccagagcggcaatgctgcacagtatcttttgtttcacttctgaat

SEQ ID NO: 4 - DNA, 300 bp, Dirofilaria immitis
  source 1..300: mol_type = genomic DNA
tcgattaaaaattatcatcgataaaattctaaaatttattttagtaaaattattattattttgatgaataagttaacaaaaaaattttaataactttttgattcgccaaaaatctaattcgttaaaaagtcgttccaaacagatatcgcttgttcgatgaaaatgtccggttgttagaaaatcataaattggttcaaataattttccagaacgttcgaaaaaatattcccttgtatcggataaataaccattacaattttccactcgtgttgcatgtgtttctcgacaaaaatcagctaa

SEQ ID NO: 5 - DNA, 300 bp, Dirofilaria immitis
  source 1..300: mol_type = genomic DNA
tcaacagaaatcgagattccaaaaagtttcctacaaatacttaattatcaatggatatttagttttgttatctgttatcataagttctgcttcttacacgattaaaaatgtccaagaattttttactattcaaatgagggaaataaaaaaccaatgccaataatatccagaaactacatacatctttcttttttcgaagctcatctattccggccgaaaacaatgaagaacattaaaattcttaaaagatagtcttagccttttccttgaccactatcttaactgtcagcgctaaaatgt

SEQ ID NO: 6 - DNA, 300 bp, Dirofilaria immitis
  source 1..300: mol_type = genomic DNA
aatagtcgtctcattactttttgacttttataattcgagaatcttatgtagtccttcactttacccttcttctgtcgaactaagaattacagcattattttcgaatttaatgtgtaaaagacaatagcagattttgtaattttgtgttaacctcactttatatttcgcttcatatcgtgacagagaattactatttcagagagtattacttgtcaccagagaatctccagaaagatttttatttacgtcggaaaatggacaaaaatggtttcttatcattagcactgatagctagtttcc

SEQ ID NO: 7 - DNA, 300 bp, Dirofilaria immitis
  source 1..300: mol_type = genomic DNA
tatctcttgttgtgtgttctgcattgtatcaaagtgggtaaattttgctttagacgttgacttattgtcttttttaagttatattctagtccatgtttttctctttgcaaatatttttttccgccgcctatgattcattgttttgtttgtaactctctattaagttgcttttagtttgaattgtatcaaaatttcaaacatttaaaatacgcactagcactattttttcttatctcaattaagcgaatcccggaacaagatttaatcgatttccgaatcacaattaaatcactggaaaac

SEQ ID NO: 8 - DNA, 300 bp, Dirofilaria immitis
  source 1..300: mol_type = genomic DNA
attttccttaacaaatcattttcaaacgaaaaaacattaaaaagtgttaaaataaaatggtgatattgataagaaattaattcaacctgcatatcaattcttgtagcggccattttcttagcaagttctatagcagctcgatccatatcaccttcttgctctaatgtcaattccggttccggaattttttttattttgccattcttcatcttttttttattttttactgatatagctatagaccctttctcccgtgcatgcctgtaggcctgttctgatatacaggcttgtgaaccactg

SEQ ID NO: 9 - DNA, 300 bp, Dirofilaria immitis
  source 1..300: mol_type = genomic DNA
ttctggggtagttatacggaaaattagacaatgaagagaatcaaaaaacatgcgattttcaaacagaggaactttggtacttttgcctcgacttactttattttaaaacccatacaaaataaatgtttcatttgattgatattgtcgtactaataattagagcttcaacattaggattttaataaccttcaatttatttcagaatttaagaaacttacgtatggatggagaaaatataaagaatggcgatgacaaataagatttgctatgaaaaaactaatgccacaagatccgaatgca

SEQ ID NO: 10 - DNA, 300 bp, Dirofilaria immitis
  source 1..300: mol_type = genomic DNA
tttatgaacaaaaataataaaaattaggataacagatatcaatttcttttagctataaatatacgcttcgattgaaaaaagctttcaaattataattaaggcatacgttacgatatagacaattaagtcgacattaattatttgaaatattttaaatttttttctctttctttttttctattctcttccaaagtgtcaaatagttatgaaattgtcagaagctaaaatgataatattattcaagtttattacctaatcttttatcacctcatttcttatcatttatctgaaaatctaatc

SEQ ID NO: 11 - DNA, 300 bp, Dirofilaria immitis
  source 1..300: mol_type = genomic DNA
atgttgaatttttaatgaaactttttcggtgcataagcattacagatctgtaagctgtgcaaaccctgtttctttgtaaattgaaacaaagatcatttattgtttccagcgtcgatttgacctggataaatgtggtaccaaaagtagatgacgagaggtaagtgcaaacaaaatgcacaaaaatgattttgatgcactcaaatcatttttaagttttgtgcaattttccattttatagtttcgtgatcggttgttattcatcaacttgattttgtttgttttttgtgacttatatttcat

SEQ ID NO: 12 - DNA, 300 bp, Dirofilaria immitis
  source 1..300: mol_type = genomic DNA
tttgacactttcagataccttacaaactcatctccagcacccaatttacaatatcgctgcctaaataaagaatttattcggatatgagactgtagttttcattccgtaccaatcatagtagaacagatctatagcatggtgtcctactaaagttgtgactggctattaagtatgtgggtgtttttacgtgtgcgtgggtgtttgtgcgtgtgtgcgtgtgcgtttctgcacatattttcgtgcgcggtgtctgtgtgtgtccgtttgtatatgccgagtgtagctgtgtgtatgttcttg

SEQ ID NO: 13 - DNA, 300 bp, Dirofilaria immitis
  source 1..300: mol_type = genomic DNA
cactcataatatacctgtcaacaaactcagaaatctgaataaaatgacgcaaaaatgacaaaaacattttatcaaccttttcttcatcactcccccgcatttccaattttcttccaaactgtttttgtcgtgctacaaagtcatcagccacttcattttcttcaagatggttcgagacgccattcttggattcaccccttatttcaactgtttccgaagtcccagcagttgaagctgaacctagcatttatatcaccacccgatgtcaaaaaatgacagcggtcagagaatacgacttcc

SEQ ID NO: 14 - DNA, 300 bp, Dirofilaria immitis
  source 1..300: mol_type = genomic DNA
gctaggtcaacagttggtttatttggacttatacgatattaaacataatatcgcctcatatacacagaaatatcaaaaaaacgaacacagctaaatcgaagaatacgaacaaatgttttaaaaattatattaaatcttttaatgctctctacaatgtcgtatcttcccttttgtctgtatttctcctttcgttccaccactgctatttctcatgcctttgaactatggttctcgttgcgtcgaattgtcctcgaaactgttgtttctgtcgaattacgtcgaactgctggactttgtcgg

SEQ ID NO: 15 - DNA, 300 bp, Dirofilaria immitis
  source 1..300: mol_type = genomic DNA
atatctcacttctgacataaattgaagtggcactgatttgaatgaaatgataaataaaataaagacgacaaggtagtggaaaaaaaaagaggagaaaacaccgtttagttttggatgcaagctcgaatctgagttttcttgcaaaccgtacactgatcaattttcttacacaaacataagaaaaaaagaagtgattttactgtagctgtatcgtataattcaaatcatatatatatatgtttcaataatctatacatttatgtatatttttttttgaatggaacagtgaatgattttaaa

SEQ ID NO: 16 - DNA, 300 bp, Dirofilaria immitis
  source 1..300: mol_type = genomic DNA
acaaatgccatcgggagagaaatatcgttggcgtactgatcacattggcggtatcacttctttgaaaactccagctggtattgtgtatcatttcatgcaatacgctatttttgatcgaatatgtcgacggcgtagtgtttcattttccaacgcatcttacgttgcgtgtatggatgatgacggacaattattggaatatcaaacaccggatcgattgcattccgtaaccttgaaacgtgacatatatgggagagtagtgcaaataacttcagatggcgaaaatattttcttcgaatatgg

SEQ ID NO: 17 - DNA, 300 bp, Dirofilaria immitis
  source 1..300: mol_type = genomic DNA
ataatatatatttccattgataatatttttcatattatgtgatgtttgaaattttctgcaattgctacattccgattaaaaacttttattatccgtactggagaattttgcttttttttgacggtttgttcaataagttgtcaatatattgtctgccttagtaaaacctttctaatctatccgttcgaattggaagttgaaagttcagcatcattcttttagtgaggtgtttaagttgttcaatagatattatttagaacgatctcaattaaaatcttctgaatgattttatgtttttat

SEQ ID NO: 18 - DNA, 300 bp, Dirofilaria immitis
  source 1..300: mol_type = genomic DNA
gcagcacattgcacacagtaaactgcaaactgaattaagagatattgggttgaattatttctaatttaaaaggatataataaatgactttgatgattgttgattttaaggtatctcggaagactccatcagtctcagtgctctagcaatcgctataggtactaaaagaaaagaaaagatgtctcgttattcactttgaaatgtacatatcaaatcattttgtcgtatgaaattaagtatattatgtctaatcgtatcattcgaaatgaatttactgtcactgttagaactatttaggcag

SEQ ID NO: 19 - DNA, 300 bp, Dirofilaria immitis
  source 1..300: mol_type = genomic DNA
agagttcaatcgccaagttgttctttttctcgctcgcagagatcaaaacggtgttggctatacactcattcatcaggctgtgatagacatctcttagaatttcagtgcttttctggatgaaaacattatttctcaaacatgacacttaaggacaatagtgcgtgacttctttgttaacgtacacgagaaaacaaaacagatgatgcttgttatcttggtgataaatgtgtattcagaataatgttatatatctttgcgtgacaaatatcatttcgttatacttcggatacgcctttttat

SEQ ID NO: 20 - DNA, 300 bp, Dirofilaria immitis
  source 1..300: mol_type = genomic DNA
aactttacttgaacttttttggtgttcaattttgaatattataccaaccattcagaagactgtatatagaaatgaaccttcaagaattaatcgaaatttttattaaaatcttttatttgaatatttcattatttaaactcattactatttgcagtatattattagatctaatgtagaaaaaaaaatcagatggcaaaaataatatcataggtttgtttttaaaattcattgcaaaattcagtgcgccgttccagtcgctcgtaattaccctatccctgagctttacaaaaagaatgcttt

SEQ ID NO: 21 - DNA, 300 bp, Dirofilaria immitis
  source 1..300: mol_type = genomic DNA
aggtatctagatagcataataaattactacacaaaccgatggaaacgcaagtttggcgttgcgtgttgatacaaaatattagagccaaggatggtatcacatgtaaaactgcaattttgctatttgtttaaagcaaataagaaataaatatttcgttcttattctttaatttatttcatcagatggctttgttataccataattgtaaatctgtcatatcttaattgcgcaatagcccaagattcttgtatattcttacatttcacaatttattttcttatttctagttttagaattata

SEQ ID NO: 22 - DNA, 300 bp, Dirofilaria immitis
  source 1..300: mol_type = genomic DNA
aatagctactcacagcttaagttaactaatggattcttgaatttatttaagcgtgtagttaagcgattaatatgatggatgcccagaatcgctttgtcttatagttttgtctcgacagaaaggatgcattgttgtcttgaatttgttcaagggaaaattaaataggtttctttcaatgactcctattaaatttttttgaatttaggcttgcattgcgtgttctgatccactattagcacgtacgggtatcgcagtgccatgtgatgcagcactatgcaaaaaccacctccatgtcacttg

SEQ ID NO: 23 - DNA, 300 bp, Dirofilaria immitis
  source 1..300: mol_type = genomic DNA
tctgttgtaagtttcacaatccagttaatttaagctcagcttatttgaaattttcaacaaaattacgaaaattactttctcggttcatttttttcaaccaccaaatatttagcataattggcctgaaatcgtcaaagtttacaaacttttgttcagcaatcttctcttactcttacaataaacatgattaacttgtcgtcataccaatctcgtttatagcaaattcttttcaaaaaaacattgctacaaattttatatcgcatcatttcaacacgcataattatttttcatatatgaaaa

SEQ ID NO: 24 - DNA, 300 bp, Dirofilaria immitis
  source 1..300: mol_type = genomic DNA
ttcacaatccagttaatttaagctcagcttatttgaaattttcaacaaaattacgaaaattactttctcggttcatttttttcaaccaccaaatatttagcataattggcctgaaatcgtcaaagtttacaaacttttattcagcaatctcctcttactcttacaataaacatgattaacttgtcgtcataccaatctcgtttatagcaaattcttttcaaaaaaacattgctacaaattttatatcgcatcatttcaacacgcataattatttttcatatatgaaaaaccatattataa

SEQ ID NO: 25 - DNA, 300 bp, Dirofilaria immitis
  source 1..300: mol_type = genomic DNA
attaactctgaacccaaagactgttggttaaaataaagatctattttagttatacatctaacattaaaggttttcgtacggaaacaagtaggtttgataattttcatgtaactgtaaagaacacctgtgaaagggatcagtaaaatttgggggatgtagcacggaaatatgaagctgagtgttttgtacccaaaagtttttcaaatctgcgaaataacgagaggtgtaatgatcgtttttaaccaaattttttgattctaatccttcccacagttttgaaattcagtaagcatttctttt

SEQ ID NO: 26 - DNA, 300 bp, Dirofilaria immitis
  source 1..300: mol_type = genomic DNA
ttgcaacaaatcaataataaaagacttgcggctaacaatatatttgattcttttttaccgttattattatgacaggtaataatagtattacaagcatatttgtaggtgtcaattttttcaattcaaattttcttaattcattatttcttcctttccttaataaatagtctttccatttaagaattaactttttgaaatctttaatgagaagacacaaaagattccggataattttgcatcatcttttctatttcgcgttagtattttatgttttcaacagatttttatgatttaactata

SEQ ID NO: 27 - DNA, 300 bp, Dirofilaria immitis
  source 1..300: mol_type = genomic DNA
gataaaatgggttcttgtcaagctcatttggcatatcttcgtcttctatatttatatcctttaatatcttctcttttttcaaattttccttcccgacgttttccatatcgacctctttcttcataaatttatcttcctcatttgcctcattttttgacttttcatccgtttcatccttatttttctttttttcatctcctattttaccttttcctttatcaacttctatcttaactttctcaatgttttttttattttctttcatctttttgttttcttctattgacatactataacaaa

SEQ ID NO: 28 - DNA, 300 bp, Dirofilaria immitis
  source 1..300: mol_type = genomic DNA
ttttacgaacaattatttcataaaagattcgtatttttgattagtttttaagaatttttttttattatttttagccaacaaatatatttttcaaaattgttaaatttgaaattataaatttcaactaaaaaaaagcaaaaagctaagccaatagaaataacatacatgtgtaatataaaatataaagtattcgaaatgaaaatcaaagtttcataacaaaaaacaaaaaatattctaaccttttagatttcatcaaaacttcactaaaaagttaaatttaaattttcaaattgttataca

SEQ ID NO: 29 - DNA, 300 bp, Dirofilaria immitis
  source 1..300: mol_type = genomic DNA
cgaacaattatttcataaaagattcgtatttttgattagtttttaagaatttttttttattatttttagccaacaaatatatttttcaaaattgttaaatttgaaattataaatttcaactaaaaaaaagcaaaaagctaagccattagagataacatacatgtgtaatataaaatataaagtattcgaaatgaaaatcaaagtttcataacaaaaaacaaaaaatattctaaccttttagatttcatcaaaacttcactaaaaagttaaatttaaattttcaaattgttatacaatgat

SEQ ID NO: 30 - DNA, 300 bp, Dirofilaria immitis
  source 1..300: mol_type = genomic DNA
tcaaagacaaaatgaagaacttaacaaaaaaaaggccaataaataaaggctatttcgtgaaaaatctaaaaaaaaaaagatctgttcctttcgaatcaagtgattcttcctactacattcgtgttgtaattcttacttgtatacagtccccagtttttcgacgataaaaaacatttcgataagtgagtttgaattaattgaattttaaaagatcataaaaataaaatcaaaataaaaagaccaaaattaagtctgataattccagaaaacacaataataaatatacaaataataaaaact

SEQ ID NO: 31 - DNA, 300 bp, Dirofilaria immitis
  source 1..300: mol_type = genomic DNA
aaataattcactaatttctcatcatcaaattatttcgtacaatcgataaatcaacgattataatagcgaagagaatgaaaattaatgtggtgcacagtatacggaccccatatacaatgttcaacagagatgaacattttttttctattaaagttttctgttcggcgaaagaaagacactttctaacgatgctttcctcccaactccccttgcaatgatagaggatgcagccaagattcgtcgactcaagcagcatcactcaaccggccatcacttcgggacctttttccctgcctttta

SEQ ID NO: 32 - DNA, 300 bp, Dirofilaria immitis
  source 1..300: mol_type = genomic DNA
cattgcgaatgaccgctatggaatatcaattagcagatattaatcgtgaattaagcacattggtggaatttttacgaccaaatcgaatttcaaaaaatgctacacttgcaacatcagcaaccattgcaacatataacagtacttcgatgcgtaatgtaaaaaagaaatgtaatgcatctgaaagctgaaaattcatctgatatattgaagcaaaaggtaagattatttttaagatatcattcttgatgctctcataatttctacatcaaatttaatcaaacgattcatttatgttcattt

SEQ ID NO: 33 - DNA, 300 bp, Dirofilaria immitis
  source 1..300: mol_type = genomic DNA
ttcttgttgtacctatcatagatgataacttaagtaccaatagcaatagtgcaacgatgcaaggattctgattaatgattataaaagtttaaccaatcttcttcattccttctaatcaagagaaaaaaaaatgagaacatttttatgacatttgaagaaaggcaatttatcgctgaaaattctactgcgatatggaagtatcagatagagaaaataaatattaaaatatggatttcatacgaaaaatgataaaagataataatttacattttggtgctttactgatatgattggagtatt

SEQ ID NO: 34 - DNA, 300 bp, Dirofilaria immitis
  source 1..300: mol_type = genomic DNA
cgatattttttggacgaatcaaacctttttgggaaatcatttgatgtcacaagcatggtttgagaaatttttttccgaattagttctgctaaaaatactccaaatgagtctagtggaattaagctaagcaccttaagtaagttgagaaaaacgtttccatttgactaacaaggctagtatatcgacatgagacagaaatggttattacttcactcacttcatgaagcgaatacgaaatatctgttcactttagtttcaatctactattttaccaataaacgtgttcttttccggataaat

SEQ ID NO: 35 - DNA, 300 bp, Dirofilaria immitis
  source 1..300: mol_type = genomic DNA
tcttaattgattttcttaactcgaaacacttgtcttgattactgtgctgtactttatcttattaaattaaataatttccatgaccacttcataccattgaccatcaaactttgatgaagtttatgtgtgaagtgccaaacaatcattcatcccttcagtttaacttattgctggtcaaattcataaaaatgcaaattatcaagcagatagtaattcagtgaacgtagcgtattctcgaaatttctttccttgtatttaccttatatagaacaacgtatatttgtagcatatattcaatat

SEQ ID NO: 36 - DNA, 300 bp, Dirofilaria immitis
  source 1..300: mol_type = genomic DNA
tttctgagtttgcgttacagcgccaaatcttcacggagatagataaaatacttatcgtgaaattttggcgccatgatttaaaaaacacggagataaaaataaaatgcttatcggtgataatttagcgccataatatgaatgaattgaaaaaacaatttgagtagaaacatgacatagagttttcgttttctggctacgaaaatggatgaatttttctggaatcgaattcagtcaaagaaataggaacgttgttactaaatgatcgaaaagctttctaaaattaaatttatgacgtctaag

SEQ ID NO: 37 - DNA, 300 bp, Dirofilaria immitis
  source 1..300: mol_type = genomic DNA
atctaaatcttcgttttatagtggtaagacttccatttgctgcattcttgcaaattaagctgttgaaaatacttttttttttgatagatttccaatttaatcatattataagaagaattaatttcgaatagaatttttaaatcatttaaactttaagttttaaaactaatataagttatgcagatttcgcgaaaaagtctcatttgttaattcaattattccaaaatgtaataattttataaattcaaatttaaactactactaacttctgaagtcaggagccagtagcaacaacgtaat

SEQ ID NO: 38 - DNA, 300 bp, Dirofilaria immitis
  source 1..300: mol_type = genomic DNA
aactttacatttatattcaatttttttttattttgtttgtttttagaaatttgaaaatgggtactaatcagtgtcatttgcagcctcttagaccctctttataacgaccgattcgatgaaatacgtcatcaatatgccagtttattgttcgggtggagaatgttttcaaaagttgctgaagtgatgaagtatagtgagaatgcaccttattcagcaccattaagaagtaaatttttgctttggaatttgacaaagacaaagcaggaagttgacaacgatgttctgatgaaacggtttcga

SEQ ID NO: 39 - DNA, 300 bp, Dirofilaria immitis
  source 1..300: mol_type = genomic DNA
gtctattttggctgtcttctaataattcattttgtaaccttttgaaatatgataaatgtagaaattttttcttcctggtctataatagtttaataatgtgttgtagtaatagttttggtgccgttgaaatatttcaatgatatgctatcgcaaaattaggaattcaaatcaaggttacaagataattcaaaaacaaacaacgtaaaaatgaaataatttcttcttcttacttaccaacaggcatatcatcatcatcctcaaattcatgactatatttaacattgtcatatttgaataatc

SEQ ID NO: 40 - DNA, 300 bp, Dirofilaria immitis
  source 1..300: mol_type = genomic DNA
cgacgcaaaaatctttcaaattgtcacccagttctctaagtgattccaatgatgttggtaaacattctgcatgatgtaccgggtaatgaactaccaagttgttttttgcttttaatacaactcgcaaagattctgaaaaccatgaaattaagaaagattaaaataatctgaactctttttttcatttttccttgaacttagcaatatactgagttggataaaatttagaaacgaaatttcgcaaatttattcagtaaattcaggaaaactcggtttcggtattctaaatataaatagata

SEQ ID NO: 41 - DNA, 300 bp, Dirofilaria immitis
  source 1..300: mol_type = genomic DNA
gtttctttggtttatctcagtaagatttgggcggaaatttcagttatacttttcatttccatgtgctgttttaaatttcttccatattagtataattttcaaataattgtagcgtcactggtttatttaaggataacaggttggactgcagtggctgagaagtgtcttgccggtcaattgtttgttggtgatcaacttgtacgagttactgatatcgacatatataatacacggcaaattccattcgttttcagtactgcatcaaaaacgggattatcggtactttgtaaatcgcagtat

SEQ ID NO: 42 - DNA, 300 bp, Dirofilaria immitis
  source 1..300: mol_type = genomic DNA
gacccctgctcacaaggcagttcccacagacaatcacacatctaatcacacacatcaactcatccgacgtaggctatcaataaggaaaattgcattgctttatcgtctaactgtaataaacatctacataatgaaattatttcgccactatgacaactaatatcgcccaatgcaaatatttgtctcagagttattcccttttaacagctgttgaacgaatagataggacgtcatgtggatgatctacttgtttcaaaggttgaggtaacacatgaaacacatgaaaacggtaatttaaaa

SEQ ID NO: 43 - DNA, 300 bp, Dirofilaria immitis
  source 1..300: mol_type = genomic DNA
aaagaatggtcagcaagatgtggaaaatcgattactatagttgaagtatgaatcgaagaggtttttttaaattctaagagaacgaataatcggcaaagagaaagttgagtaaccttattttgccttgttttcagtcaatttataatatgcggttaattgtgttaaagaaagtacaaggtatgaaatctaagccaagaaataagagaaaacagctaatgattatttctgcattttttctttttcgacacaaacttggaaccagaatcaattgaactagtaatcagattttgattattgctt

SEQ ID NO: 44 - DNA, 300 bp, Dirofilaria immitis
  source 1..300: mol_type = genomic DNA
ttagattttgctgaagcattgttggttagatcgatgaaaatataattatgagagattttgttgaaattcagcaacaaaattattattcatgtcttcatgctgtcagttttgtttttatttcttctttgacatcggttatatttttgtcttccaacaatataaaaaaaaaattataatcaattggtaatcaaattaaaactctaattgttagctccctaaatcagctttaaaaaaataattgcttaattggtatttgctactattagcaaactgaaactatccttttctcgaatggtgaac

SEQ ID NO: 45 - DNA, 300 bp, Dirofilaria immitis
  source 1..300: mol_type = genomic DNA
atgagctgatatttgatatgcatattaaaaatagggtaaattacattaagttagatatcgttcggataaattaattagaaaaaatgtttaccaattagatcgcaatgatgtaaaatttcacgtatttttattcttaagatttatttgcaaaattcaaaaatatgtcttatgaaaaataatatttctgtgtaagaacaagggaccgattcacttgatttattcgcaaacaatcgaaattcaaaattagtaattttaaatattgctttattcaaaccataccaataataatttgagagattt

SEQ ID NO: 46 - DNA, 300 bp, Dirofilaria immitis
  source 1..300: mol_type = genomic DNA
attgattgattcaaataagaaatttaaattatttcccctttttttcaaaagatttaacaaatattatttatttgatctcctcgttcgttcttatctttttgattatcaatccatcctcctccatcatatagctaatttattttttgcatcgtaaatcaattgatgtatgattgatttcttgattataaaaagttagaagaattgaattgcttaaatttaattattgataatgaaatattattatatttcaaaatgatacgaagaaatatgacgatgataagagaaaatatgatatttatc

SEQ ID NO: 47 - DNA, 300 bp, Dirofilaria immitis
  source 1..300: mol_type = genomic DNA
tacgataagttattttattttacacatctccatccttgactagtgtccgtgccgactgtcggacttgaaccgacaacctactaattacaagtcagttgctctacccaattgagctaagccggccatctagaatgtgcgaccccgtcgtggtacatcttctataatcgtttggtattcaggactctcttctttcgtgggtggaggatcttgatacagttgactattaaaaatagggcctttgttagtctgttacaactcatagacaaaggcgacaattttagcttacatcttacgttatgc

SEQ ID NO: 48 - DNA, 300 bp, Dirofilaria immitis
  source 1..300: mol_type = genomic DNA
atggtagaaaattatatgaaaaaatatcatactaaaaatataacagattgttataaggtatggtttaagaatttacaacaattgattatttatgataaaaaaaaaaaaagtaaatcagtgaatcattaagatagttatgataagcagtttgtattcggtaaagcgaatgattagaggaattatgggacgaaacgtctataacctattctcaaacttttaatgagtatgacgtgtcttgcttgcttaaaattatttcaatgatcatttcactttaccagtatgatcatgattagacttgaa

SEQ ID NO: 49 - DNA, 300 bp, Dirofilaria immitis
  source 1..300: mol_type = genomic DNA
ttagtatcgatattatcacaaatgatatcactttcatcaatactggatacgattttattagtatcataattttgtggctcgcattccgaaagttttacacgtagaagattaacctgcaatatgatttattttatcattttcgaatatccaactttgaaataattcgaaaatgttgaaaaattttgaaaaattgttaacaaaatattacaaaaatatcaaatgaaattaaataactgtccatttcaaaaaaagaagaaaaattatgaaattaccaattaaaaacaggacttattaattaaa

SEQ ID NO: 50 - DNA, 300 bp, Dirofilaria immitis
  source 1..300: mol_type = genomic DNA
tgtggaaataaagtacaattaattgctgttcgcttaataatattattttcattcttggctttttttttctttccccgtgatattataaaatatagttttttaattttaacaaatcgtcataattatttaaaaaatactgaggtgagtaaatgtaattggttgctggaaaaaaagtgggtgatgagaggtgaatgaaagcagaatagtttatgattgcatcaaatttcctccttaatctgtgattaaaatcaaacaaaacccgaaaagtttcttcttcgcctttttcttctctttgtttca

SEQ ID NO: 51 - DNA, 300 bp, Dirofilaria immitis
  source 1..300: mol_type = genomic DNA
cgaaatccgccgcgtgcattactttgcgcttgttgattacgacgcatttgttcgtcgttgataaccttatcaatcatcatacgtccgttacgtatgcaatcaacatcgccagttaggctgaaatcaaatggatggcgatgatatcaaaaacaaaaataaggagtatttgctgaatcatttctttttctgtattattatcaaaattttctcctttccattgtttccttcttaatcaagtgaatgctcatttcattttgaaataatccaacgtaataattccccatattcccaattactttc

SEQ ID NO: 52 - DNA, 300 bp, Dirofilaria immitis
  source 1..300: mol_type = genomic DNA
agaaatattaaactttgaaaagatgtgacatgttctgtaacaaaagcccaaaatttcgactgctgcggcttgaagtaaaattttggaatatgctacatcagtagtgcaacagatggttcgataaatagtggtaagtgatgggaatcctaggaatagatgggaattgtatttcagatataaatttgatgcatattttcatagttgattatatctacgatcacacgttgaatattctaaaagcaaactgtaattaactaattgaatttgaaaatttccaagaattaaaattggtaacaaaaa

SEQ ID NO: 53 - DNA, 300 bp, Dirofilaria immitis
  source 1..300: mol_type = genomic DNA
attgtcaggaatgagaagcaagttttggatacttaagggatgaatggaacacatacatggcagaaaatgttagtaatcaaaccatttaaattacttagccactatgctaaactttctagaagtatggttgaacgtttaaaaaccttcgcaaaaattgtattagattatcttaatcttccctacatcaaaacagagaatttttgttctacgacgtgagtctgcatgtattaaggaagttcgtatcatgacgtaaatatcctgagtgattattgaattcagaaaatgagctttttcatttgg

SEQ ID NO: 54 - DNA, 300 bp, Dirofilaria immitis
  source 1..300: mol_type = genomic DNA
atatgagtgttacatgtgtacgttacatgtaaatattatatgttatatgtaaaaatgtcatgtatagcatctattcacgtgtacgtacacgtgtatatacatatacattgatacttaatacgtatacgcatgaatgaacagatattatatatttacgtacactagactcacatgtacctctgtatacgcatacatgtacagatatatgtttgacatacgtaaattcatatatgcttttatttatgcttatattaattgtcacatacatgccttatattttcgttgttataaacacataaa

SEQ ID NO: 55 - DNA, 300 bp, Dirofilaria immitis
  source 1..300: mol_type = genomic DNA
gaaaataaaattagctgaaaatatatgcgaggtaaagcacacagaagaattaacttaaggtaatatattgtaagaatttttatattcggcgcacctaataatttttagaccgcatatgcccagtatttgaaactggtagcgctgttcgtacttgctgttgtccatgttatgtatatgataccattcctaaatacttttgcggctgtggtttccagtgttgatgtgactggtatgatgcctaacactggatccttccatctgcggcattttgttgaaattcttattgatgtgagctgttta

SEQ ID NO: 56 - DNA, 300 bp, Dirofilaria immitis
  source 1..300: mol_type = genomic DNA
caactgtgaatcataaacattacttaaattaatgaagctagttaacgacaaatatatttttttatgtatcagtgctatcatataacataaaaacttactttcattaataaatgagctcaaatattgacttttgtccaaaatgctcaaaatgtcgtcataatatttgaaatgaagataatttcacgcttttcgaagcctcctctcacgtcttttaatcttcttttcttcttcttgctctaatggttctgcgaaaaaccacggtgcaataatcactttccataatttatacagtacataagc

SEQ ID NO: 57 - DNA, 300 bp, Dirofilaria immitis
  source 1..300: mol_type = genomic DNA
ctgcttaactcttttcatttttcagagaatcttctctaaaattgtgaattgatccaaaccaaagaatatggataatgtgattcgaattcctggaatttagattttgagagttttgaagtttttaaagagattgaatttctgtgaccttctggtatatttgatgtcatttcgggatgcgtatttttgccgaaaatttttggcctcactgcaatcttgttaaaagtcaaaaaaattcaatcgtagaatttcgggtttacctgatattactggaaatctctgatctttgttctagattgctgt

SEQ ID NO: 58 - DNA, 285 bp, Dirofilaria immitis
  source 1..285: mol_type = genomic DNA
ataaagaatttgcaactctgtatacctttttgcagtgcaaaagcggatgaattcttcactgcagtgtgacagattcctttgataaaattgcttcgttcttatgtaaacttggaaattctcggtagttatgcttttgctagttgaaaatgttctgctcttgtaaaacatgcaaaaagagattatctttgttctattatggaaagattcttttgaaattttgacgactgagaagacaaattttatcccaacttgtcatctgcaataaaaatttttcctgacctgttt

SEQ ID NO: 59 - DNA, 300 bp, Dirofilaria immitis
  source 1..300: mol_type = genomic DNA
aaaatcaaatcaatatgatcagataactcatacttatcttactgaaaattcctcattcaagggaaataaataattgcaattcttgattccgatcatggatgattttcaagcaaattaccaatgatatctatcgataacgattacagcatacagctataacttattattgattgaattgatgaaaataattttaccagaaatttatcaatgtttatctcattgcagtatacgatgtttagtgtgacaacactttttcttggaataattgtgcataaatcattgattgcatttagtattgga

SEQ ID NO: 60 - DNA, 300 bp, Dirofilaria immitis
  source 1..300: mol_type = genomic DNA
tcctgcccacattctttctactttagataatcaacaggagttagttgaaagagaagactaggaacagttgcaacttctgaatctttctgactttctttcgttttgtaaattatttatttgtataaatttaaaattcgaagagaaataatccaaggtccaacttctttttctgttagttcttgcgaatgctccatcaaaatgcaaaaatatgattagaattctgatggaaattaacaaaatcgattagataagaaaagtacaaaacagaaactaactttttctcccattttcatattatag

SEQ ID NO: 61 - DNA, 300 bp, Dirofilaria immitis
  source 1..300: mol_type = genomic DNA
tcattgctttaatactttttaacgagaattttctcgatcaaaataagatctgcaattgatatacgtcaataagcgaacattagctgtattacacgctaatattcacatatgatgaacgttgtaagcgtcatacatcaacatatatccatccgataaataatgaccactacacattgctaccaaccatcctatcccgccactatttgaaatgaactgagaaggagttatcgacacaggcttcctagcaaccaaacaaaagacgagacagatgaatagatagacagacagacgaacatacaa

SEQ ID NO: 62 - DNA, 300 bp, Dirofilaria immitis
  source 1..300: mol_type = genomic DNA
agattctggttattattgtatttctgatttatttaatcccaacttaaagattcattggctattgtttagcatctatatcaattttataaataaatagtaatacctgatgaaaagcaataaataattagatgcaaattttaattagatacagtttgatggaaaacattgaagccatgtacaactaatttatgcatgttgaattatgcatgcataattaatttatgcatgacagcaagtttggtataaaattaattttgtatgaagataaaattttataaataatgataataatgctggtaa

SEQ ID NO: 63 - DNA, 300 bp, Dirofilaria immitis
  source 1..300: mol_type = genomic DNA
attattgaaaagaataatgtagctaattagttgaagctgttaaaagtaaagctaaaaagatgatggaaattattcgtataaacattctttgtaaacaaacagtcatttctgtgaataaacaattataattataaacaatacttttcaagacaataaaaaaattaggaagcattgttgtgataatcaatagttgatagactgtcaatgtatttttatcagtcgtgctgctttttttccctttcttgactcatttattttattatttattgatagaatgtcaatattctagtcatttgttat

SEQ ID NO: 64 - DNA, 300 bp, Dirofilaria immitis
  source 1..300: mol_type = genomic DNA
atcttaacttgctttaaacaaataaattaaaacagcccaatgttccaagaaaaaaagataagttaaaagtggggtgtccaaaaatttatgaattgaattggacagttattcagatcctgaaaatacgcttctctgatcactgcaaatattcccgataaataagtgaacattaggttaatcttaattttcccttaactttccttagccttttttaaatttttggattattcaagcatttttattgcggtatcgtttttgtaaaaaaaaaagtataattcaacattcaggctcgacgttatg

SEQ ID NO: 65 - DNA, 300 bp, Dirofilaria immitis
  source 1..300: mol_type = genomic DNA
aattaataaaaagaaaggaatacgataaaatatctattttttgaaactaatcaaacatattcctcactgctcaccggatagttgctttctaattttacattaagaaatatatttttttttttcaataaggaaagttatgcagactaggagaattctactctgaagaagagataagcatgttagaattattaaaatctatggaaatatccttaaaagaatgcctatagtagctctgatttcgaaaaaaaaagcaaaaaacaaaataacaaattctgctcaattgaaataaaaaactttcct

SEQ ID NO: 66 - DNA, 300 bp, Dirofilaria immitis
  source 1..300: mol_type = genomic DNA
taaaatatctattttttgaaactaatcaaacatattcctcactgctcaccggatagttgctttctaattttacattaagaaatatatttttttttttcaataaggaaagttatgcagactaggagcattctactctgaagaagagataagtatgttagaattattaaaatctatggaaatatccttaaaagaatgcctatagtagctctgatttcgaaaaaaaaagcaaaaaacaaaataacaaattctgctcaattgaaataaaaaactttccttcaacttccagcatcactgctgtga

SEQ ID NO: 67 - DNA, 300 bp, Dirofilaria immitis
  source 1..300: mol_type = genomic DNA
aactgctaaaaaattgaaactagtgttagattgataagtgggcagattaaaaccaattgtgttattggcccgttaattagtgactctgaatagctatggcgaatcgtatagtgttgtaccgacgacgtatctatcaaatgtctgccttgttaaatttcgatgatagtttatgtgcctattatagttgtaacgagtaacggagaataaggtttcgactccggagagggagcctgagttgccacattcaaggaaggaagcagtcgcgaagattacccactcttagaatgaggaaagagtgac

SEQ ID NO: 68 - DNA, 300 bp, Dirofilaria immitis
  source 1..300: mol_type = genomic DNA
gaaaactaagaagtaagtgaaatttctaagttctttcccagaaaggttagatccaatatttgttttcattttagcatttttatccaatgaaaaatgtgcccaataaatacttgtatatagtattgcatttaaaaacttcagaaagcacaatgagatctaagctcagaaatatgacgaataccaatccttttcctagtcttaccgcttcttaacttttgtgtcgctttataaaaattaaaaataaaaagttgaacaatgggaattacatcattttcatctgaatggtttatttcctattct

SEQ ID NO: 69 - DNA, 300 bp, Dirofilaria immitis
  source 1..300: mol_type = genomic DNA
cttccctagctatgccttttcgtcacttaagcttcnnnnnnnnnntctagctacgtatcgttatcatttatgcttctttagctacgtttctccatcatttatgcttcctaagctacgtatcttcatcacttacgcttccctagctatgtcctttcgtcacttaagcttctttggctgcgtgtcttcatcattaatcttctttagctacgtatcgttatcatttacgcttccttagctacgtctttccatcatttatgcttcccaagctacgtattttcatcatttatgcttccttagata

SEQ ID NO: 70 - DNA, 300 bp, Dirofilaria immitis
  source 1..300: mol_type = genomic DNA
gatcttaaaattctatgaaacttcttctgcatggtattgtttccaacagaatataatgacaatagcaacagtattggttatataaaaatattgactgcagcaggattatatttcaagttcttttaatttcatttatttattctttcatttacttttactgtttttatgtttttcttctttaaaaaatatgatttctctcactgttctctttcatctatctatatttatttgataattgcttatatgataactagctaaagggaaataaactttcagtcatcatagcttcattttagtaaa

SEQ ID NO: 71 - DNA, 300 bp, Dirofilaria immitis
  source 1..300: mol_type = genomic DNA
ctatactaatcagtccactatccatttttaggttgcaaaagttgcaatgacggtttgatttcatcctccaatgcaattttgagtctcaatctcgagagatagatcgatcgcttttagcttgatttagcttggttaatgttgtgagggatattgggcagaaattctgtcaagcgttacttaatgaaatagtaaatgatcactgatatttattgttaatgatacttgagctctctagattatgaactggaaggttttcgatagaaataatcgatacatatattagaatcgacttcttttttc

SEQ ID NO: 72 - DNA, 300 bp, Dirofilaria immitis
  source 1..300: mol_type = genomic DNA
tcatctttttcacatttcatttaatcatcattttatcaattcctatttttaaacaaattcttttcaaatattctctctttccttctctttttgttttccgcttattcattctaatgatgaacagatgtagaaaatttgcattctattgctcactacaattttgagtagaatatatttaattatttgattcgagacagatggttatagcctttagcttcagcttctcgttcaaattaagtacttgtgacctttccaagtaccattaaagctttcctgcgtttcctaattagaaaaaaaagg

SEQ ID NO: 73 - DNA, 300 bp, Dirofilaria immitis
  source 1..300: mol_type = genomic DNA
gcattttaagttaaaagtatcacgctgcatgacacctcacgtttgctatctcaaattgagtaggttagaatctttttttggctactattcaaatattaataataaattgctgcaaacagatttcacaccggaaaaaaattaaatttttctagcaatgttttaactcccttattaaatatttatagaaaatcgactacttaaaaagaattgactaacatttctgaatctctgcagagatttatagatggattagcatcctacaagtttttatctttttgctatatttccattattttttta

SEQ ID NO: 74 - DNA, 300 bp, Dirofilaria immitis
  source 1..300: mol_type = genomic DNA
gataagacgtcttattttgtaataattcaaaaattaattaatatagaagtaagatcttgataataattaatatgctcaaatttcttaatgagaatatgttcaggatgaagatgaagtgaaagaaattgatagattgaggaagcaattgctaattgaaacagaacagctcgtttccaattctcttaaagatttactgaagaaaatttattatccacttgaagaagctattgatctcaaaattcatcagaaattaattcaacaaattgctgccttgttgaagtgtattagtatcttggataa

SEQ ID NO: 75 - DNA, 300 bp, Dirofilaria immitis
  source 1..300: mol_type = genomic DNA
accgcaaaatacctaaaaatttctataacaacgattaacacggcctcgaactggaagcatattaatccatgcgtggctcaaacttcaatcataaagacaagatctagagatcaacacaaaatggtgaattgttaccctatcgttgctaaagtttgagagaaaaaagtgctaaatcaagtagtacaccaaatttagttaatattaagaaatcaatttagtactgaatttaaacaaatgaaattttacgataaaataaaaaagtacctgatcaaacagcgtcctcccgttattcccattgct

SEQ ID NO: 76 - DNA, 300 bp, Dirofilaria immitis
  source 1..300: mol_type = genomic DNA
tataagactagtaaacagatcgtaatataataaatatcgattttattttaaattttcgaaaacttccaaatctatcgatatgaaattaaagatcaatttttaatttccataatatatttagattctatcccaacatcactcatctttatgtcaacttatttaattctcttattaacattatatttcttgtttacaatgataaattttatcaattttctaatatgatagaacatcttcatcatctgaagatatgcttttctcatctttgtaacaattcgtatcgcttctgattttactttc

SEQ ID NO: 77 - DNA, 300 bp, Dirofilaria immitis
  source 1..300: mol_type = genomic DNA
gttttattattgcttattgaatagtgataataacactttgatatgatattgttttgttgcgatcattgtattgattataaccttaattaaacgaggatattatgggaaatgtatttattacaaaattaaatatgaaaggttgaagtcttgacgaaactttcaaacacatttctcgaattttctctgcaaaaatatcgttacgatttttggaaattatgaagtccaagaattcaatcgagagttcgccatgtcactttggctagtttcgtttgtttttaatatttcaatcaaaagtcaatt

SEQ ID NO: 78 - DNA, 300 bp, Dirofilaria immitis
  source 1..300: mol_type = genomic DNA
ccttggatattgttcttgacatcgttgatcagaaggtcaccgtagtgttcggtgagcgagatggaattggactcaggtttattctccgtttttttcatgtttttgaattttagagagaaaataatgtttgtctgaatggttagcaaactaattagtttttaagttatcaggaactcgaagtatcttcttttgcacttctttaacctttttcatcaaattttttaacagtaacaagatttttttgagaattttcaaaatatttttgacttctgatgatatttgatgagaaaaccatcactg

SEQ ID NO: 79 - DNA, 300 bp, Dirofilaria immitis
  source 1..300: mol_type = genomic DNA
agagtattattatacatgatgatgatgatgatgatgatgatgatgatgatgatgatatgatgatgatgatgatgatgatgatatgatgatgatgatgataatgataatgatgatgatgatgattaattgcttatttttaatgattgataactttaaaagaaatcattgaaatttgatcgaataaaaattttcttaaaaaaagcatttgctatttatatagtaaacctataaaaaattacttatttttattactaatattcatttgattgtatgaaagagaagagaaaaaaaacctttgca

SEQ ID NO: 80 - DNA, 300 bp, Dirofilaria immitis
  source 1..300: mol_type = genomic DNA
tggtatcacagcactgggtttaatttcaacaatcggttgacgatcttttcgggatatgcctatacccagaaatgaacgtatgccaaacgatggtatgtttgatgcaacagacgacgtcaacttaaaatgtgtttttttttcaaaaattcaatatttttagtttaaaattgcacgtcagtaaaaattaattcataataaatctctttgatttcttcgttctccttttttttcagaaaaaattgaaattttacatacctgatttccaagagcatataaagcatcacttaaagcattctgcga

SEQ ID NO: 81 - DNA, 300 bp, Dirofilaria immitis
  source 1..300: mol_type = genomic DNA
tccttttcatgatttgtagctaaccaataagatgtgtatatgttcatatatttactctcccctgactcttttacactctcattctctcatttgttcatttagataagtaatatgcgcctttctcttcctgattctctcaatctttcatcccttcatctcctcaatctttctcccattctctcaatctttcctgcattgcattcattgatgaaacacgatagtattaataagcataatttgataaattgaaataattttttttnnnnnnnnnntcattctctcaatctttcctgcattgca

SEQ ID NO: 82 - DNA, 300 bp, Dirofilaria immitis
  source 1..300: mol_type = genomic DNA
tttgaattaacaaaatattaacaattacaactatttcggaatttaatttaagaataatttaattaatcaatttcctattttgtattttaaaaattaccacaataattatgtaatttttgggatatttgaaactttgaaaaaagtggtattgtatttgagaataaattaattaatgtaattcttgctgctcatcgttccataacttacaaatatttctcggtattttatttgagataattcttatcatttcttccatagctttcaatatatttataacttatttgtaatcactcttatcac

SEQ ID NO: 83 - DNA, 300 bp, Dirofilaria immitis
  source 1..300: mol_type = genomic DNA
ttgagatatcaaatcaagcgttgcatatttatagtacactggtgtagctgaaatcgcgaagagaacacgaaaatcagagaagtcaatggttcctttgtgttggatttcacatgaaagcatccttatgttgtacatgcgtgattacaatatgatacaagatgtaagctaaaaattgttttatctttgtctatgagatgtagttcatactctataataaagtcccaacccttaattctcatattcacaaccgtatcagaatccaacaccaaaccattataaagaatgttcttcgtcgaggcg

SEQ ID NO: 84 - DNA, 300 bp, Dirofilaria immitis
  source 1..300: mol_type = genomic DNA
ccactatcgcttacactttctttatcctgttcttcttcatctttcgttttggactttattttactgtcaggtgacaagcaaagtaacgatgttggactttgcgaagatgtggatggtacgctagaaaaaaaatgaggattggttaatatgtctaattattacatcgcttttttttaaatcttttctaaaattaaactgaataatcaacttatttgctattcagtttatcttattttttatcaacaaaattcgaggaaacaaatcgcttatcagaataattgttttgatcaacaaataaag

SEQ ID NO: 85 - DNA, 300 bp, Dirofilaria immitis
  source 1..300: mol_type = genomic DNA
caatcccacaaattcagtgtgtcggcgggtcagcgaagggaaagtttgaaccgagggtatgtacaaattgtgataattttgtgatgacgtagtaaatttcatagttttgcatgctttaatgttgatagtcgcacaatcctacgttgattaaatttagctattagatatcctactaaattatgttgttcataatttttgtttttaaaatgctccacttatattttcaggttgtgcagtgctacaataggggttatgacggcaatgatgtccaatgggagtgtaaagcggaaatgagcaatc

SEQ ID NO: 86 - DNA, 300 bp, Dirofilaria immitis
  source 1..300: mol_type = genomic DNA
tcagataaattgtatttgatgttaattcaaagaagaaaaaaataatcagtagaatatgaatcgaataatattcatacaaccagtttattcattattattcacttttaacgtctaaatgacgtagctacgctttttttctcgctttcaagcctttactgaccaagattaatgtacattctgttgaacaagattaatcgacattctatcgatcaagatcaagcttttactgatcaagattaataatgacattcttctgttgatcaagattaatcgacattccattgatcaagattaatcgac

SEQ ID NO: 87 - DNA, 300 bp, Dirofilaria immitis
  source 1..300: mol_type = genomic DNA
ctctctaaaacctattggtcactaaacttgcactgactaaaaactattggtcatcagacttgtgattcattgaaaagaccgttagccgctaaaattatgattcactaaaaaaaatctattgatcattaaatctgtaatcattgagaaactacaatcattggtcattaagtttgtgctctctaaaacctattggtcattaaactgactaaaaactattggtcactgaacctagagtctattaaaaaaaaaatcattgtatcaataaatttattgtttactatcaaatccattgattactga

SEQ ID NO: 88 - DNA, 300 bp, Dirofilaria immitis
  source 1..300: mol_type = genomic DNA
tctaaaacctattggtcactaaacttgcactgactaaaaactattggtcatcagacttgtgattcattgaaaagaccgttagccgctaaaattatgattcactaaaaaaaatctattgatcattaaatctgtaatcattgagaaactgcattcattggtcattaagtttgtgctctctaaaacctattggtcattaaactgactaaaaactattggtcactgaacctagagtctattaaaaaaaaaatcattgtatcaataaatttattgtttactatcaaatccattgattactgaata

SEQ ID NO: 89 - DNA, 300 bp, Dirofilaria immitis
  source 1..300: mol_type = genomic DNA
aaaatgtatcaaattcttcgatgccataaattatacagacttgattggcattttttctaactttcatcatgaaccattctatttctaaattgatccattacaaaatcaactttgtgatatcatcaatctcagtcataacgagaaataatgataatataaagcgactatcatttgaatttcctgaatattcaagatgtaattacatcttttttttaatgtaatcaaaatttcttgccatcaataatttttcaacatatgctttcatcgactgccttatgcagatcgtaatgatgacagcca

SEQ ID NO: 90 - DNA, 300 bp, Dirofilaria immitis
  source 1..300: mol_type = genomic DNA
attgattaaaaagaatcaacattaaatttttgatatagtcgagaaatccttcgtgataattcttttagaacaattctttacactaaacttgtatttacttgcttattatttgtctaaagatactaactatttgtcagtggaatttatgatcttggcattattgcatataacgctttcctaaaatctgaaatttttcagtattttaaaaactaagacgattattaaatattactcaaagcttagaactttgattatactaatcaaatcaaaaatttcatcagcgatttttgttgtgtcatt

SEQ ID NO: 91 - DNA, 300 bp, Dirofilaria immitis
  source 1..300: mol_type = genomic DNA
attttttccagcagaattgtcatcaaaaatcccatttttgatatcctcttcatcgaaacttgctcctgaatccagagaacaacgaagaatgtgtaaatctatttcagtagcctgctcattgtgcaattcagcgactttatttctgtgcttcaagctaacttcttcattatgccactcctcttctctcgctattttttcgctatctaattcaaaatcttcgtctgaaacggaatcaactcctgacgatgtactcgacactgataatattttcatgccgatttttctctcaaacgaatcttt

SEQ ID NO: 92 - DNA, 300 bp, Dirofilaria immitis
  source 1..300: mol_type = genomic DNA
gaatgaagagcaaaaaaatagtcacgaccacctgcaataaaaacagcatctccgtaaaaatgattgaattgattcccgaaatacgagtttatcaaattgagaattatgcaaattaattatcagcatgcagatttactgattttatatctctcataccgaaattaaggtgatgttttccatttctttgtttccacaatgtcttctttgtgaatcgttttggatcaactattaatccgatcgaatcaatcctccaaatatgagtttattcaacgtaacaaaacattgtccgagataatcaaa

SEQ ID NO: 93 - DNA, 300 bp, Dirofilaria immitis
  source 1..300: mol_type = genomic DNA
tggaaatttcgaaatcgaaaggatgaagaaaaaggatccttgatctatacattaaatatcaccatatcaactagcatggcaagtcaaagtaatgttatcatttaaataaaaaagatgaatagtaggactacaggttatattgttaaaagtcgacaaatttggagtaattgacagagatcaacgattaaatgtaatggatgatcttatcttcttttttcaactacgccaaaatgaaaataacaattgaatttgtcgaataagaaactaacattttgaaaataagattgaacatttataaat

SEQ ID NO: 94 - DNA, 300 bp, Dirofilaria immitis
  source 1..300: mol_type = genomic DNA
ggttggatcattatcgacagaactttagaagtttcttgataaggacgaaaagaagcagcaccattgctgatctaaacaaggaaaaaagaccttttttggaatattgaagtttttactgataggtgcgtgctgtgtactgtgggcataagtacaagcttcatgctccgcagcgtgaatacgtgctgcatgcatactatgcagtaaaggtgcgtgtcgtattgctcaataagtgtataaattgctgcttttcttgcatagttaaatattttgttttcattttttccgctattcaaaataaat

SEQ ID NO: 95 - DNA, 300 bp, Dirofilaria immitis
  source 1..300: mol_type = genomic DNA
gttgggatttcagactctcactcggtgtcgtttcacagtgatatctgaatcgaagtcacaagcaggtatgaatgcataacaactaatatccattgcagaaacaaggcaaaactgagaagctcgagcaatatagctatagaagctggtaccacagatgacattacatggtatttccatttcagcttcacaaacattgtaaatagcttgcttcgatgattcaatatctcgttctacgatattcttaaagtaatttttatttatttgaagtatagattacatccatgttctatctatcatttc

SEQ ID NO: 96 - DNA, 300 bp, Dirofilaria immitis
  source 1..300: mol_type = genomic DNA
tgttctgaacatctctttttgattatcttttttaattcctccattattttcgtttttttcgttgtgaattaatattgtttgtctttgattcagatgatattttcggatcgtaaatagatggcatcggcataagcgtattgagaagcattcaatggtgcactcttgcttcttttttttttgaaatctttctcgataatcaaataagtgcaggatgccaatcattaacaatttcgttccactttttcagttcttattcttataacaccacatctcatttgcaattttgtcgccaatgatttt

SEQ ID NO: 97 - DNA, 300 bp, Dirofilaria immitis
  source 1..300: mol_type = genomic DNA
ttttttcgaggtcactctggaaaaataaatcatattttaaaaagacataaaataaaaaatatgtatatataagaaaatttttactctgaatttcttaagaaaattctcgattctgttttccataaattccggaatatgttgtccctgaattaagaattcgattccttgcacaccattatttcgtctagttcctgtgtgaacaatgtaacctggaaatgaacacataaactgtaatattttgagcttaaaataattatgaggatgcgaaactgaagatattcataaatgtttaaaaaaaaa

SEQ ID NO: 98 - DNA, 300 bp, Dirofilaria immitis
  source 1..300: mol_type = genomic DNA
gtccatgcattgcttttcggaagttagtgtagattcagtgaatatttaataccagtctctttctaattcaaaagagcctcccatttcttttttcagtttcagtctctgaatcagagcgtgtaatctaccactccattgccgaaaacagctcgatgtatttcctgctacgtagtgtttagaattggcgtatgccacttgctcattattcgcgcatgaagtgtaactgtgaatagaatgatactactgttagaagagaatgcgttcactttatttaacattatactgattcatttcttcttt

SEQ ID NO: 99 - DNA, 300 bp, Dirofilaria immitis
  source 1..300: mol_type = genomic DNA
agtgaacgagaaaaaacagaagaagagatagcacatcaagatcgtgagaaattaattagacaagaaaaagctcgtcttacacaaatatatcaggttttctttttcttgctttcgaaagttatttgaattatctcatttctttgaattttataagaaataatttaatttttttttgaaattttgcctattgagctctaaattttgtaaaaagttttctaggatgatgttagcaaagcaaaaaagaaatccaaaagtgatggtaacaaacaggaagattttatagtgaggtacgataatacg

SEQ ID NO: 100 - DNA, 300 bp, Dirofilaria immitis
  source 1..300: mol_type = genomic DNA
tagacaatatcatccttcctttttttttgctcaatttctctgctcattgctttgatgataatggtaggtggtataatgaaacgaatagataattgatgttcgcaaacatttgctgttaaatttcagtaaagaaattgacctttttgctttgtgttggatgtttagcttcattttcttcttgttcattgtcatattcattctctcaaaacttcttgcttagcgatgctaatataaatactggaagaatgcctttgctttgttttagttgtaaatcatcaccaaggtatttttttgcaaaat

SEQ ID NO: 101 - DNA, 300 bp, Dirofilaria immitis
  source 1..300: mol_type = genomic DNA
aagatgaaactaaaaaaaattatttcgaaaaaaagaaaataaaattaatgaaataaaagcaaaaatgaacaaaccgtattaattttaaacaataaacaatatcgaaatcgaaaaatggactattattgatgaactatattttcaaaatgtgaaaggtcaaagtttgtttcaattatgataaatacaatttaaaataagattaagctaacaaataagttgagcaaattgatgaaacaaacaaatcagaatatattacagaaaatgatataacatgaaaatatattagaccaattattttta

SEQ ID NO: 102 - DNA, 300 bp, Dirofilaria immitis
  source 1..300: mol_type = genomic DNA
ttgaagttttcagataaactttgataaaaaattgttctatgaattctcaaatttcaattagtgatacttatttcgaaggtaattatgcctgattgaatcttcaatatcaacaaaatgaaaattttagtatgattgttaactcatacacctctaattaaaggtattttctttatcccatgaaatgaaaatttattaagaacttagaaagctacggtatgcctttgatgcaaaagaaagattcattttcattaaatcatgtttaaaaaaaagagcaaagagcaaaaggtgatgaaagttttt

SEQ ID NO: 103 - DNA, 300 bp, Dirofilaria immitis
  source 1..300: mol_type = genomic DNA
ttctatacgaaatatttgtctgccataaatctactcaggaactcgatacatcaaaacataagtacgcttgctctttatttttcgtttgaaaaataaatagatcattttcgcacttacatttcaatttcaattgctttattcatatctttctgtttttacttactggtatttaacagtcgttgttcacaatttaatgatctatgaaacaccatttaattgtatttggactaacttttcgacaagcaaaagattaaaattgtcttcagatacagttataaatttacattgaagataaatgaa

SEQ ID NO: 104 - DNA, 300 bp, Dirofilaria immitis
  source 1..300: mol_type = genomic DNA
taacgatctgtatatcaatggaataatattcagttcatgttgtactcgatatgagatagaattacaattttggaacaagataatctcaacagctattttcaagaatagttaaattaggataccattcaaagaaactttaaaaaatgatttccatacattaatgctttttgtgttttcgctctcgaccagaatccaggaattgtccattatcatcaatttgattaacttttatctttattctaattcttcaacatttctctaattgatattagtttcaatattttaataagtaaaaattta

SEQ ID NO: 105 - DNA, 300 bp, Dirofilaria immitis
  source 1..300: mol_type = genomic DNA
ataatgtgttattgatcaaaggatttttagttacctaccagatggaaaaaaagcaagtttacgaaaacagaagttagcatcaactttcatccatggttacaccgtatataatccaatcgactcatactttatgttgatctgattttatagcagataactagttaccttgctcagcagcagctaaatcctttctatttgcttaataacagaaatatttttcattaacaaagaaattatactccgtgtttgacatttcattttaatttcgttccaaaaatgaaaaaagcttcgtccggaaat

SEQ ID NO: 106 - DNA, 300 bp, Dirofilaria immitis
  source 1..300: mol_type = genomic DNA
attattttgtagtttttcattttttagttcaattttcctttgcttattttaaatatgccattctttattcagactcatagcgaatgcatatgttcattaatttttttagttacagttacaaattctcaatttctctttaatcatttttttttccaaaaatagtctgagcactcaaccattcattcaacaattgcagctttttttattggagccttgtcaaattatcaattcgtttccatgtttattattgaaataataaacggtatttaggataacgaagttcgcttagcttctttgact

SEQ ID NO: 107 - DNA, 300 bp, Dirofilaria immitis
  source 1..300: mol_type = genomic DNA
aaaaattcaggtaatgagatcagtaattttttttggtcactttgctgtttcttatcagctcattgttatccatatcaaatgagcgaaagtgtgtatcacatattggcagagtgtaatctatgaagattttgcgtatcaaagtaattatgagagaactgataattttattttaaagtagtagaaaactcgaattaagctaataaataatcggttgatatccatgaaatgaattactaatgaaatggataattgagtaataacaaatgatattcatgaagaaaggcaggttttttttaatag

SEQ ID NO: 108 - DNA, 300 bp, Dirofilaria immitis
  source 1..300: mol_type = genomic DNA
tatacttaaaacaagaaatacaattaatgccaatagcagagtgaaacttctgaaaaataatgagttgaaactggtaaaattaacattttattagaaatttcagaaacttatgactcctcatggcactatcacaaaatgtttgaaaaaaattgacagctcgcgtcgattgcaaaaatcatgattcctgatatttagtatcgaacatgtgacaaataatataaagacctaaccataaagcactgaaacaactcgcggaaacaaaaaattaatttgcataaacacggaatacgatcagaaaat

SEQ ID NO: 109 - DNA, 300 bp, Dirofilaria immitis
  source 1..300: mol_type = genomic DNA
gaatttttttagaaggcttgaagtcgagaatattagagactatatcgaagacttaaataatcctggtaatcttctgtatgaatcaaaattacctcgaacagaaccattcagcacatcacgagataattcatggaatgaaactagccaatcagagcgttgtaaaagaagaaagttatgaaatgaccttaaaatcaatttaaagcatgtcctcgccatataagcgttgaaaagttaggatagaatcaattatcaaaaaaatatgttaactagatcttatcaatcaaaacatcagaaggaaaa

SEQ ID NO: 110 - DNA, 300 bp, Dirofilaria immitis
  source 1..300: mol_type = genomic DNA
atatgataatagtgaaacaattccatcacaataaatattatcgattaggagataaattaacattgatgcctcaattttggtcaacaatatatatttgctattagcatttttattaaatcgtttttatctgacttgacataaattgaaatagaaaaaattgaatctgttccttgttagattttcttctaaaaattcttgaaatacaaataatttcttaaatttcaatatttctacataatgtattgcgacaaaaatgctaatgattggcttattattatttcgaataattttttaatcaaa

SEQ ID NO: 111 - DNA, 300 bp, Dirofilaria immitis
  source 1..300: mol_type = genomic DNA
agctcgaagatcggacaaaatttgttcagcttgttgccttgaggctttagtctgaaaagacacttaaaagtataaacaaattatattcaaaaaatcttattttgcatttgcgtcttaatttttgctttttgcaaagttttttccgagcaagtttttctatcttcgaaaagattatatcaattaaaatttcaatttaagcaatcattgcctcttcgagtttctgtttcagcaaataaatatcaccaccacgacgctgtcggaagaaagaaacgcctttcccaatttctcgtctcaactttt

SEQ ID NO: 112 - DNA, 300 bp, Dirofilaria immitis
  source 1..300: mol_type = genomic DNA
taagaaagctgggagattttccaaaaacactatttcccacgatttgttgttttctatgatcaattcttaatcaaactctgaaattctcaaattttcgatttctatccaacttctacatatttttttagaaaattcatatttagcaaagctgagtgtagaaataattcatacttgcaattcatttttcttaaattttcgaatttcttaaaaaagtatttcaaattacctaccaattttgattggaaaattcgtggatgctaaaaattcaaatcaaaatagttaaacagtattcctaattgt

SEQ ID NO: 113 - DNA, 300 bp, Dirofilaria immitis
  source 1..300: mol_type = genomic DNA
aatttaaaaaacacatcgacattttgcggtacggtaatgattgtttacagtaactaaatgtgtcctacggtagtaatactcgtgtacgtaatgaatgagtatagtgaccggatatttccttcactagtaggcaatattaagaagtattttcattttcatattctatctaaaataaaccgataaaatggtttttgaattattactttttcattgttattttttgatcctaaattgtaaaatactgtaataatttagctaatttctatgattctattcaatatgcttaaattaaaattctaa

SEQ ID NO: 114 - DNA, 300 bp, Dirofilaria immitis
  source 1..300: mol_type = genomic DNA
tcgtatttgttgtatgtaatatagaaatattgtttaaattcaatatgtagaaaaaatttctannnnnnnnnnaattaattacatattaactcgtatttgttgtatgtaatatagaaatattgtttaaattcaatatgtagaaaaaatttccataataaagacgaacagcatttataattatcaatgataagttgaaattaattcatcaatgataagttgaaattaatttatttgaaataatttctttgaaattcgaatatagacgagaatttttttttttttgctaatcgtttatcaaat

SEQ ID NO: 115 - DNA, 300 bp, Dirofilaria immitis
  source 1..300: mol_type = genomic DNA
tctagcaatataaattacaagaatatgccgtccaagtatttcagaatttattattaatttggataataatacattgtaaatactgcgtattctggattattatgcactgcataataacatgcaatttcgtctacatatcgcgaataaacgccaaaagatttctcgataaaagaaaatataagaattcgtaaatgaatgttgtgtcagagatatgtgttaattcataagtcaagatgttgtaaatcgatccatattagtaatcatatttacgtgctcgtaaataaaagcggtgattcttgt

SEQ ID NO: 116 - DNA, 300 bp, Dirofilaria immitis
  source 1..300: mol_type = genomic DNA
atcgaaaaaagatgatctgatgacggaaggcgaaatgtctgcagaagctaagatgacggaagaaaaaagtgaagaaatgaaagaagaagctggtaaaactcagaaggaatgtaaaactggagaatcgaaaaaagatgatctgatgacggagggcgaaatgtctaaagaagctaagatgtcggaagaaaaaagtgaagaaatgaaagaagaagctgataaaactcagaaggaatgtaaaacggaagaatcgaaaaaagacgatctgacgacagaaggcgaaaaatctgaagtagatgagcc

SEQ ID NO: 117 - DNA, 300 bp, Dirofilaria immitis
  source 1..300: mol_type = genomic DNA
actaatgataagaaacggagccgacgattttaggaaatgaataataacgacattgacaaccattgttagaaaattgatagtactgataataaaagctagttatagaaaattgataataataataaaattgctggtagcaaatgtctagaagtgataataaaattaatgatagcaaatggattagcaatgataattaaactgatgatagcgaatggattagtaatgataataaaattgatgatagcaaatgactaataatggtaataaaagttaatgctagtgataacttgtattttaagt

SEQ ID NO: 118 - DNA, 300 bp, Dirofilaria immitis
  source 1..300: mol_type = genomic DNA
acagtttatagttacaatattctccggtgactaactgtattttacaacttataattatagattacaaaatatattatagtagttttataattacagtattcttaagtgaataactatactttacagcttacagttacagtagttttctatgtttttgaatattaattttacatggtttttcctagtttcagtttcaaaattttcagatattttatgtgttaaagcaaattatattcgagatataaaaagtactggtcatatcttacaattctcatccttctatattggaaagaattgagt

SEQ ID NO: 119 - DNA, 300 bp, Dirofilaria immitis
  source 1..300: mol_type = genomic DNA
gtattgggaccgcgtatcgggaaatctgaaagaagtctttaacagtattttaaatgaataattcaaatcgttacttcttaatatattaatttatgcgtatatatgcagtacatagcattgcttaaattcttatttttccgcggttaaaaccctatgtaagataagggaggtgattgtatctgcgccgtactccttgttttaatctacctgcttgttgtatatcctccacatattgtaactgcagcttcacatttgcatatatagtaagggcatcgttgtctccagaagagatatattatc

SEQ ID NO: 120 - DNA, 300 bp, Dirofilaria immitis
  source 1..300: mol_type = genomic DNA
gctgcccgaatgttacaattaggacgaaagtaaaagtagttgactgtaggtatgacgataaaggaaaaatttgtatcttaagactttacaatttctaaatattacgtgttttatcgtgctaacatcacgaattccatattcacaaaaaaaattttgtagaactccatctggtttggatgaatttgctacagttgaactggatgatggaacgaaattgcaaacatctcttattgttagtattttctaaattctgtgaaattttgcaacggcattcatgtttaattattaatttggagaaag

SEQ ID NO: 121 - DNA, 300 bp, Dirofilaria immitis
  source 1..300: mol_type = genomic DNA
aaataagcaaatccgaaagtattacatatacggactaaatattgccattcattcgggagtataccattgcaaccattggtatttcatttgatcgagaaaactagtttttgtagtttgggataaagagaaatggagagaggaactttcatgatcaatttctttacgtactgaaattcatttctatggatgttctttttctatttcattctcctcagcaaatacagtccgaacagtcatcaaataagtctaaaaggcatgaataatataaacatcagcaactttttaaatgaatgcttatta

SEQ ID NO: 122 - DNA, 300 bp, Dirofilaria immitis
  source 1..300: mol_type = genomic DNA
atttctataaacatctcttgcattgattaatttaacatgttgcaataaatatttcttacttttgaatgtatcatttactagaaaaaacttcaatcgaggaaataagttttaaaataaattcatatttgaattcatgtcagttcaaaaattctattactataatacatgtctcttggttgtatctttttttcttttgaaataatacaatcaaacggtttcctaaattttcatagacatcatattttaaaaaaaaatgcatttgaaaattttcgaaaatcaatgaacttaattgatgaaaaa

SEQ ID NO: 123 - DNA, 300 bp, Dirofilaria immitis
  source 1..300: mol_type = genomic DNA
gcatgtgtatgtagtatttctttgtaaacaacatatctaatctgtctgtccctttaacattatagaatagtcagttagtccgctatttattttaataacaaaatatctcacttaacttccatttctttcctaaataattttgtttcgctagatctttcctataattttcaaattttcaaaaatgaattaatcttttatttatatatgtgtatgtatgtgtatgtatgtatgtgtacgttgcatatatgtatatgtatgtgtgtatgtgtgtatatgtatatgtatatgtgtgtatgtgtg

SEQ ID NO: 124 - DNA, 300 bp, Dirofilaria immitis
  source 1..300: mol_type = genomic DNA
tatgcataatgtgcgaccagccaataatgtcttcaaaccataattatgcagaaataaattttttccagaaataatttttttttttttacatatacttccgatctgtgagaaaatacatttgaagtgaagtgtgaagcaatgctactttttcaaacaacattgtgaaaatggattaaaacgcaccaatggagcaagagatcgtaagtttcgttccgcatgtcctgtggcaacgtgtaaaccatccgttaacgatatatgatgtaaaagccgacacacccaaattaaaatccattataaaca

SEQ ID NO: 125 - DNA, 300 bp, Dirofilaria immitis
  source 1..300: mol_type = genomic DNA
aaatggatcgtattcacttcgtaagaacttagtgaacgaaaaatcaaaccatcacaataactttactttttttctttttttactaaacacactatcctatgaaaacaaaatgtccaaatagattcatatgataatgaactgtgaagttatccaatctatcagttctcgaagagggaataaataaaaacattaagcaacccaccgatcttcgctgaccatctccttcttcattagcaagaagcaaatcttgtggtgatatttctgcaaccatctgcaaaataaagcacgaaaaattaagga

SEQ ID NO: 126 - DNA, 300 bp, Dirofilaria immitis
  source 1..300: mol_type = genomic DNA
tttgatatgcaatcaactaaccaaatcagaattcaatgcattctgataaatttcttcaatatcgtgcatcaattcgacatcatattttgacagtgatgctacctttttagccgtatttcggaaaaatatgaattcaaccagctgcgtcccaaaatttaaggctgtagcaagtccagcaacaaccagccctacaactgaaaattctaaaaactggttcacgtgcttatcattaataatttcaacactatcactatctccacatgaacttgatcgattataatttagtagaactgaaaaaaa

SEQ ID NO: 127 - DNA, 300 bp, Dirofilaria immitis
  source 1..300: mol_type = genomic DNA
acaaattcgttttaatattggattacattgaaattgctgaaataaagtggaaatattgaaaagcattttacaatatttgttaacaacattatatttaaagaatatacaccttggtttaaatggtaaaataatctcaagaattttcattaggttaatttttttttatttatttatattcacaaaaaattgtaaaagaaaacaaaaacaacaataataacggtgacaacaacaacaataataataacaaaactatttgttgtgattttgcagcattgatgtagtggggatcttttggagcga

(19) United States
(12) Patent Application Publication
(10) Pub. No.: US 2024/0240252 A1
(43) Pub. Date: Jul. 18, 2024

(54) MARKERS TO PREDICT MACROCYCLIC LACTONE DRUG RESISTANCE IN DIROFILARIA IMMITIS, THE CAUSATIVE AGENT OF HEARTWORM DISEASE

(71) Applicants: Elanco US Inc., Indianapolis, IN (US); McGill University, Montreal (CA)

(72) Inventors: Roger Prichard, Quebec (CA); Catherine Bourguinat, Quebec (CA); Timothy G. Geary, Quebec (CA)

(21) Appl. No.: 17/877,830

(22) Filed: Jul. 29, 2022

Related U.S. Application Data
(62) Division of application No. 15/887,164, filed on Feb. 2, 2018, now Pat. No. 11,414,703.
Application No. 15/887,164 is a continuation of application No. 14/896,736, filed on Dec. 8, 2015, now Pat. No. 10,000,811.
(60) Provisional application No. 61/839,545, filed on Jun. 26, 2013.

(51) Int. Cl. C12Q 1/6883; C12Q 1/6888

(52) U.S. Cl. CPC C12Q 1/6883; C12Q 1/6888; C12Q 2600/124; C12Q 2600/136; C12Q 2600/156

(57) ABSTRACT

Disclosed are nucleic acid molecules from the genome of Dirofilaria spp. nematodes that contain single nucleotide polymorphisms related to reduced responsiveness of the nematodes to macrocyclic lactones. In one example, the species of Dirofilaria is Dirofilaria immitis (the agent of heartworm in animals). Also disclosed are methods for determining the responsiveness of Dirofilaria spp. nematodes to macrocyclic lactones, methods for selecting a treatment to treat an animal infected with Dirofilaria spp. nematode, and kits for determining the responsiveness of Dirofilaria spp. nematodes to macrocyclic lactones.

The present application is a divisional application of U.S. patent application Ser. No. 15/887,164, filed Feb. 2, 2018, which is a continuation application of U.S. patent application Ser. No. 14/896,736, filed Dec. 8, 2015, which is the U.S. National Stage filing under 35 U.S.C. § 371 of International Application No. PCT/US14/44000, filed Jun. 25, 2014, which claims benefit of priority to U.S. Provisional Application 61/839,545, filed Jun. 26, 2013; all of which are incorporated herein by reference in their entirety.

### REFERENCE TO SEQUENCE LISTING SUBMITTED ELECTRONICALLY

Incorporated by reference in its entirety is a computer-readable nucleotide/amino acid sequence listing submitted concurrently herewith and identified as follows: as a 8 kilobytes xml file named “78045-366383_ST26.xml”, created on Apr. 13, 2023.

### FIELD OF THE INVENTION

Disclosed are genetics related to macrocyclic lactone (ML) endectocide resistance in nematode parasites (e.g., Dirofilaria immitis). Single nucleotide polymorphisms within the genome of D. immitis are disclosed that, singly or in combination, correlate with reduced responsiveness of the parasites to MLs. Also disclosed are methods for detection of these parasites, methods for treatment of these parasites, and methods and kits for determination of responsiveness of these parasites to MLs.

### BACKGROUND OF THE INVENTION

Dirofilariasis is a parasitic disease of animals and occasionally in humans, which may result from infection by a species of Dirofilaria such as D. immitis, D. repens, D. tenuis, D. ursi, D. subdermata, D. lutrae, D. striata and D. spectans.

Dirofilaria immitis (heartworm) is a parasitic nematode that commonly infects dogs, foxes, wolves, coyotes, and cats. Heartworms may cause serious vascular damage and may be fatal, especially in highly active animals.

The life cycle of D. immitis is well known (reviewed in McCall et al., Adv. Parasitol. 66:193-285, 2008). In brief, a mosquito may become infected when it draws blood from an infected host (e.g. a dog). In the mosquito, microfilariae (mf) develop to the infective larval stage. When the infected mosquito feeds, it may transmit larvae to a new host (e.g. another dog). In the new host, the larvae continue to mature for eight to ten weeks, after which time they move to the right side of the lungs and the pulmonary artery, where they become adult. Adult worms mate and females produce eggs, which develop in utero into the long thin embryos (microfilariae) that are released into the bloodstream. A mosquito that takes in the circulating mf when it draws blood from the infected host starts the cycle again.

D. immitis may be found wherever its vector, the mosquito, is found. Generally, D. immitis may be found on a world-wide basis, but are very common in areas with mild and warm climates.

Macrocyclic lactones (MLs) are often prescribed as therapeutics or prophylactics in the management of D. immitis in veterinary applications. Example MLs include ivermectin (IVM), milbemycin oxime (MO), moxidectin (MOX) and selamectin (SLM). However, resistance to MLs is common in a variety of parasitic nematodes and appears to be developing in D. immitis. A number of tests have been described for the detection of anthelmintic resistance in nematodes of livestock and horses, including, faecal egg count reduction test, the egg hatch test, microagar larval development test and molecular tests based on benzimidazole resistance (reviewed in Coles et al., Veterinary Parasitology 136:167-185, 2006). Prichard et al. (European patent EP 0979278) describes a P-glycoprotein sequence in Haemonchus contortus which may be useful for the diagnosis of ML resistance in parasitic nematodes. However, there remains a need for methods to detect D. immitis (heartworms) that are resistant to a ML.

### SUMMARY OF THE INVENTION

Genetic variations (e.g., SNPs) have been discovered in the genomes of Dirofilaria spp. nematodes that relate to reduced responsiveness of the nematodes to macrocyclic lactones. In one example, the nematode is Dirofilaria immitis (the agent of heartworm in animals). In one example, the macrocyclic lactones are ivermectin, selamectin, milbemycin oxime or moxidectin.

Methods for determining the responsiveness of a Dirofilaria spp. nematode to a macrocyclic lactone are disclosed. In one example, the method involves determining the genotype of the nematode at a polymorphic site in a nucleic acid molecule that includes one or more of SEQ ID NOs: 1-127 from the nematode. In one example, the nucleic acid molecule possesses at least 80% sequence identity to one or more of SEQ TD NOs: 1-127. In other examples, the nucleic acid molecule possesses at least 90% or at least 95% sequence identity to one or more of SEQ ID NOs: 1-127. In one example, the nucleic acid molecule includes a fragment having a length of at least 100 nucleotides of one or more of SEQ ID NOs: 1-127 and includes the polymorphic site. In another example, the nucleic acid molecule includes a fragment having a length of at least 50 nucleotides of one or more of SEQ ID NOs: 1-127 and includes the polymorphic site. In one example, the nucleic acid molecule includes a fragment having a length of at least 100 nucleotides and that possesses at least 95% sequence identity to one or more of SEQ ID NOs: 1-127 and includes the polymorphic site.

In one embodiment of the method, the presence of an alternative nucleotide at the polymorphic site in the nucleic acid molecules indicates that the nematode is likely to be resistant to the macrocyclic lactone. In one embodiment, the method may include isolating the nucleic acid molecule from the nematode, and optionally purifying the nucleic acids prior to determining the genotype of the nematode. In one embodiment of the method, the genotype of the nematode is determined by DNA sequencing, hybridization-based methods including with allele specific oligonucleotides, microarray analysis, enzyme-based methods, single strand conformational polymorphism (SSCP), high resolution melt (HRM) or approaches based on PCR, RT-PCR, or qRT-PCR.

Isolated nucleic acid molecules comprising one or more of SEQ ID NOs: 1-127 are disclosed. In one example, the nucleic acid molecule possesses at least 80% sequence identity to one or more of SEQ ID NOs: 1-127. In other examples, the nucleic acid molecule possesses at least 90% or at least 95% sequence identity to one or more of SEQ ID NOs: 1-127. In one example, the nucleic acid molecule includes a fragment having a length of at least 100 nucleotides of one or more of SEQ ID NOs: 1-127 and includes the polymorphic site. In another example, the nucleic acid molecule includes a fragment having a length of at least 50 nucleotides of one or more of SEQ ID NOs: 1-127 and includes the polymorphic site. In one example, the nucleic acid molecule includes a fragment having a length of at least 100 nucleotides and that possesses at least 95% sequence identity to one or more of SEQ ID NOs: 1-127 and includes the polymorphic site.

Kits for determining the responsiveness of a Dirofilaria spp. nematode to a macrocyclic lactone are disclosed. In one example, the kit contains a probe capable of determining the genotype of the nematode at a polymorphic site of one or more of SEQ ID NOs: 1-127. The probe may be an oligonucleotide, a primer or an aptamer. Using the kit, the genotype of the nematode may be determined, for example, by DNA sequencing, hybridization-based methods including using allele specific oligonucleotides, microarray analysis, enzyme-based methods, single strand conformational polymorphism (SSCP), high resolution melt (HRM) or approaches based on PCR, RT-PCR, or qRT-PCR.

Methods for selecting a treatment to treat an animal infected with a Dirofilaria spp. nematode are disclosed. In one example, the method involves determining the genotype of the nematode at a polymorphic site in a nucleic acid molecule that includes one or more of SEQ ID NOs: 1-127 and selecting the treatment based on the genotype of the nematode. In one example, the nucleic acid molecule possesses at least 80% sequence identity to one or more of SEQ ID NOs: 1-127. In other examples, the nucleic acid molecule possesses at least 90% or at least 95% sequence identity to one or more of SEQ ID NOs: 1-127. In one example, the nucleic acid molecule includes a fragment having a length of at least 100 nucleotides of one or more of SEQ ID NOs: 1-127 and includes the polymorphic site. In another example, the nucleic acid molecule includes a fragment having a length of at least 50 nucleotides of one or more of SEQ ID NOs: 1-127 and includes the polymorphic site. In one example, the nucleic acid molecule includes a fragment having a length of at least 100 nucleotides and that possesses at least 95% sequence identity to one or more of SEQ ID NOs: 1-127 and includes the polymorphic site.

In one embodiment, the method involves treating the animal with one or more alternative agents when an alternative nucleotide is found at the polymorphic site. Alternative agents may include one or more of an arsenic-based therapy, diethylcarbamazine, and antibiotics. In one embodiment, the method may include isolating the nucleic acid molecule from the nematode, and optionally purifying the nucleic acids prior to determining the genotype of the nematode. In one embodiment of the method, the genotype of the nematode is determined by DNA sequencing, hybridization-based methods including with allele specific oligonucleotides, microarray analysis, enzyme-based methods, single strand conformational polymorphism (SSCP), high resolution melt (HRM) or approaches based on PCR, RT-PCR, or qRT-PCR.

### BRIEF DESCRIPTION OF THE DRA WINGS

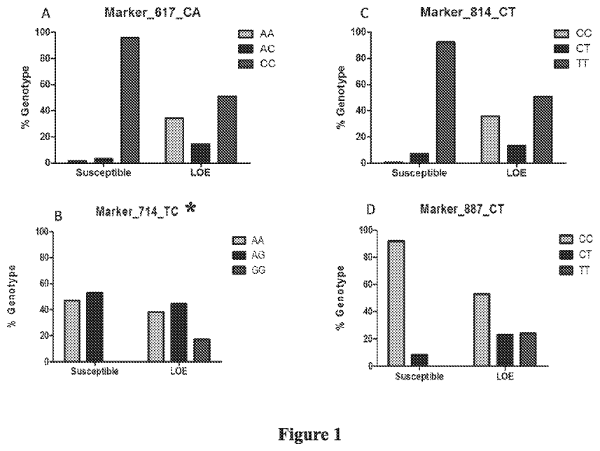

FIGS. 1-28 illustrate the genotype frequencies for the SNP within each of the indicated markers, for susceptible and LOE isolates. The graphs are representative of markers that are also designated as SEQ ID NOs: 1-109 within the application. For markers designated with an asterisk(*), the genotype indicated shows analysis of the reverse complement of the sequences shown as SEQ ID NOs: 1-109 within the application.

FIG. 1 illustrates the genotype frequencies for the SNP within Marker 617 (SEQ ID NO: 1), Marker 714 (SEQ ID NO: 2), Marker 814 (SEQ ID NO: 3), and Marker 887 (SEQ ID NO: 4).

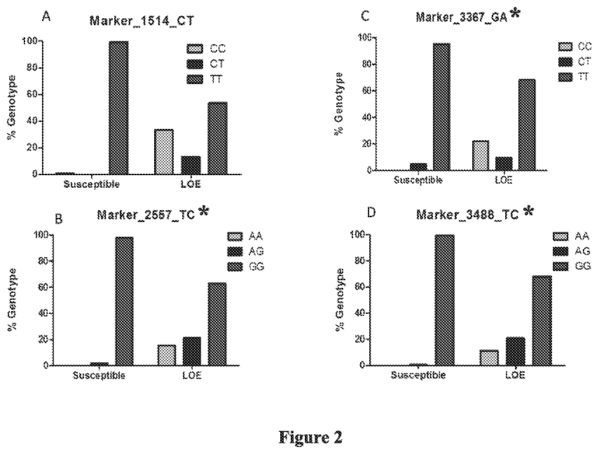

FIG. 2 illustrates the genotype frequencies for the SNP within Marker 1514 (SEQ ID NO: 5), Marker 2557 (SEQ ID NO: 6), Marker 3367 (SEQ ID NO: 7), and Marker 3488 (SEQ ID NO: 8).

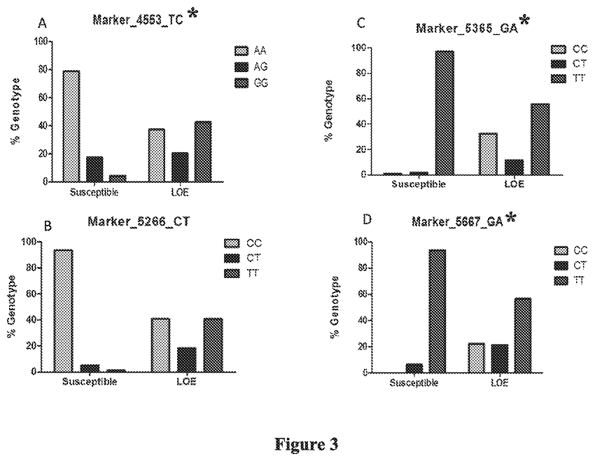

FIG. 3 illustrates the genotype frequencies for the SNP within Marker 4553 (SEQ ID NO: 9), Marker 5266 (SEQ ID NO: 10), Marker 5365 (SEQ ID NO: 11) and Marker 5667 (SEQ ID NO: 12).

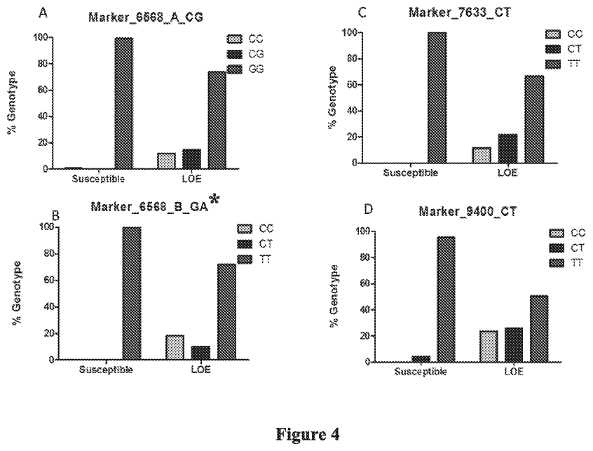

FIG. 4 illustrates the genotype frequencies for the SNP within Marker 6568_A (SEQ ID NO: 13), Marker 6568_B (SEQ ID NO: 14), Marker 7633 (SEQ ID NO: 15), and Marker 9400 (SEQ ID NO: 16).

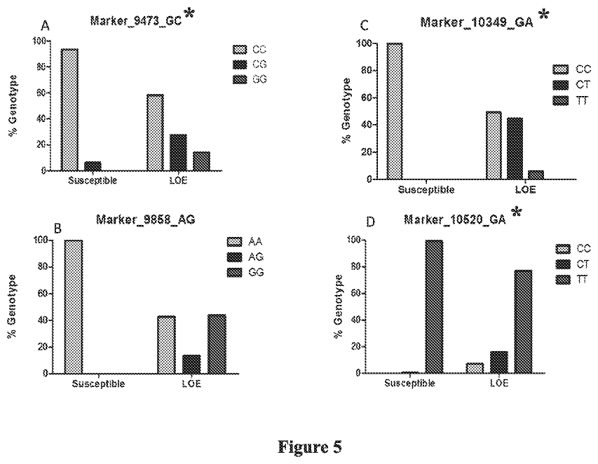

FIG. 5 illustrates the genotype frequencies for the SNP within Marker 9473 (SEQ ID NO: 17), Marker 9858 (SEQ ID NO: 18), Marker 10349 (SEQ ID NO: 19), and Marker 10520 (SEQ ID NO: 20).

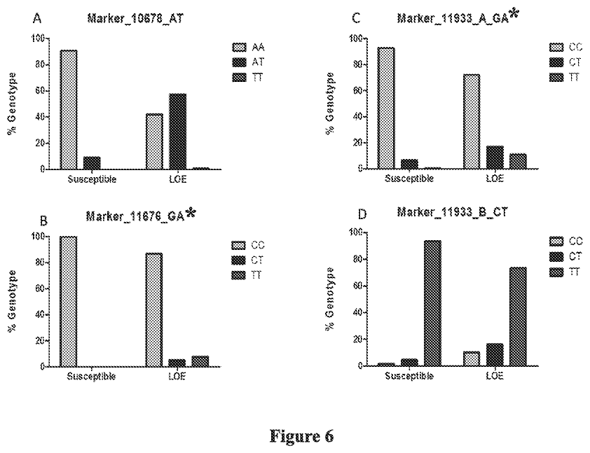

FIG. 6 illustrates the genotype frequencies for the SNP within Marker 10678 (SEQ ID NO: 21), Marker 11676 (SEQ ID NO: 22), Marker 11933_A (SEQ ID NO: 23), and Marker 11933_B (SEQ ID NO: 24).

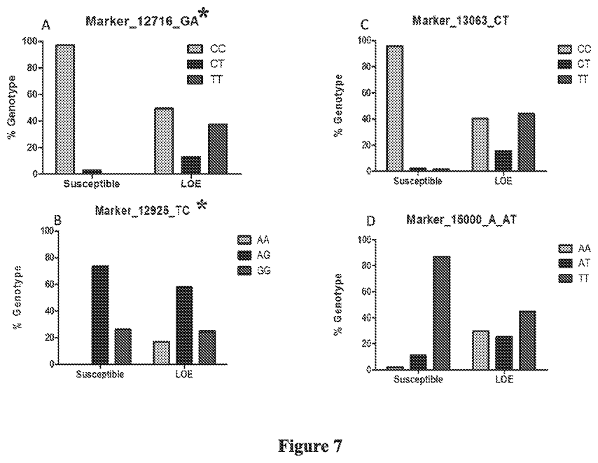

FIG. 7 illustrates the genotype frequencies for the SNP within Marker 12716 (SEQ ID NO: 25), Marker 12925 (SEQ ID NO: 26), Marker 13063 (SEQ ID NO: 27), and Marker 15000_A (SEQ ID NO: 28).

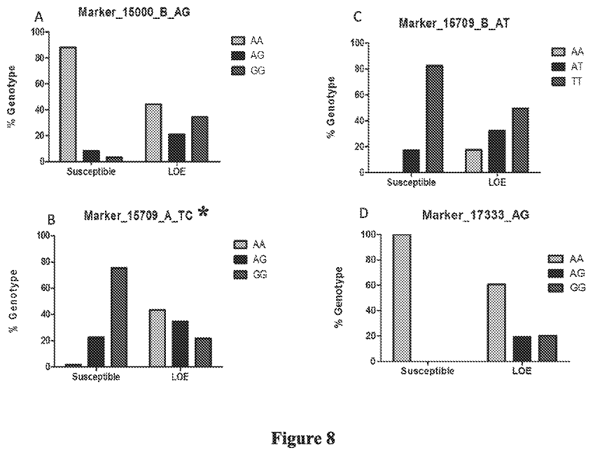

FIG. 8 illustrates the genotype frequencies for the SNP within Marker 15000_B (SEQ ID NO: 29), Marker 15709_A (SEQ ID NO: 30), Marker 15709_B (SEQ ID NO: 31), Marker 17333 (SEQ ID NO: 32).

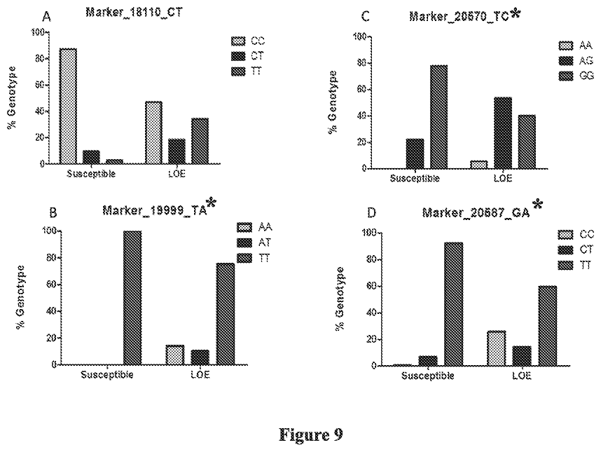

FIG. 9 illustrates the genotype frequencies for the SNP within Marker 18110 (SEQ ID NO: 33), Marker 19999 (SEQ ID NO: 34), Marker 20570 (SEQ ID NO: 35), and Marker 20587 (SEQ ID NO: 36).

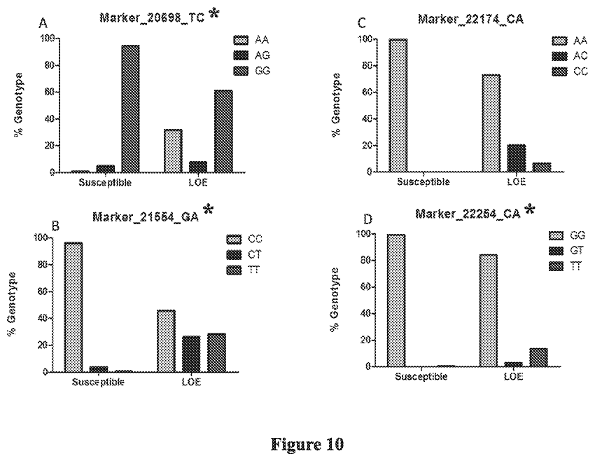

FIG. 10 illustrates the genotype frequencies for the SNP within Marker 20698 (SEQ ID NO: 37), Marker 21554 (SEQ ID NO: 38), Marker 22174 (SEQ ID NO: 39), and Marker 22254 (SEQ ID NO: 40).

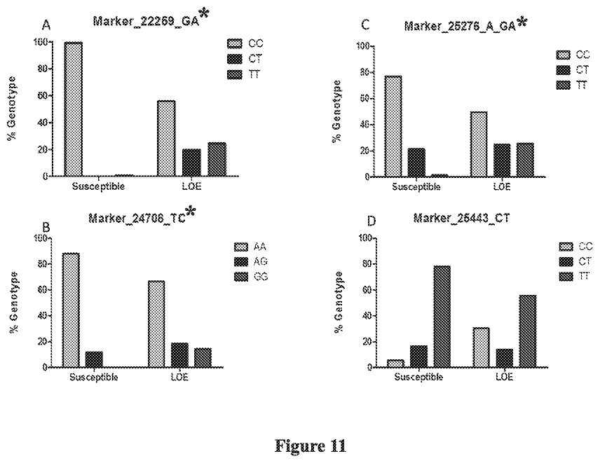

FIG. 11 illustrates the genotype frequencies for the SNP within Marker 22259 (SEQ ID NO: 41), Marker 24708 (SEQ ID NO: 42), Marker 25276_A (SEQ ID NO: 43), and Marker 25443 (SEQ ID NO: 44).

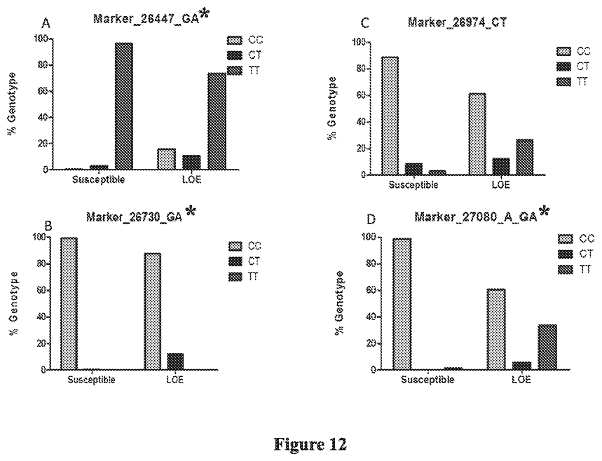

FIG. 12 illustrates the genotype frequencies for the SNP within Marker 26447 (SEQ ID NO: 45), Marker 26730 (SEQ ID NO: 46), Marker 26974 (SEQ ID NO: 47), and Marker 27080_A (SEQ ID NO: 48).

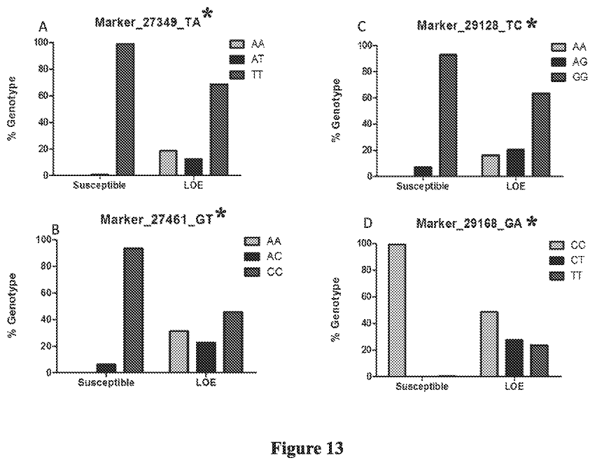

FIG. 13 illustrates the genotype frequencies for the SNP within Marker 27349 (SEQ ID NO: 49), Marker 27461 (SEQ ID NO: 50), Marker 29128 (SEQ ID NO: 51), and Marker 29168 (SEQ ID NO: 52).

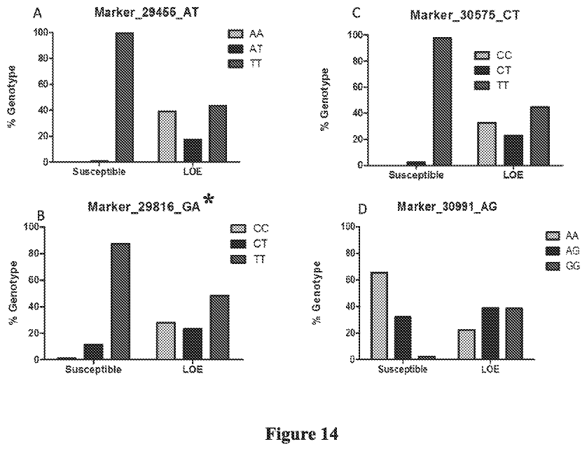

FIG. 14 illustrates the genotype frequencies for the SNP within Marker 29455 (SEQ ID NO: 53), Marker 29816 (SEQ ID NO: 54), Marker 30575 (SEQ ID NO: 55), and Marker 30991 (SEQ ID NO: 56).

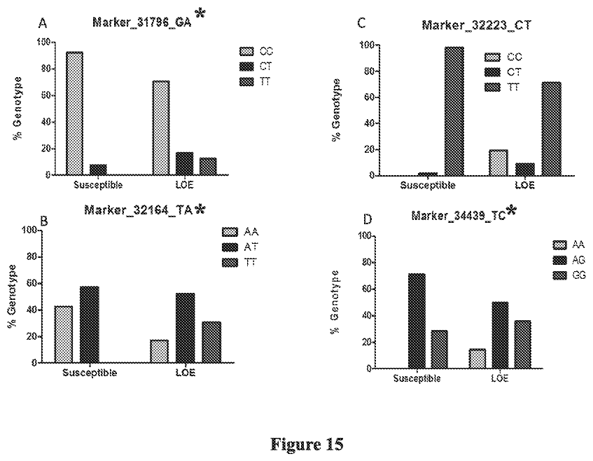

FIG. 15 illustrates the genotype frequencies for the SNP within Marker 31796 (SEQ ID NO: 57), Marker 32164 (SEQ ID NO: 58), Marker 32223 (SEQ ID NO: 59), and Marker 34439 (SEQ ID NO: 60).

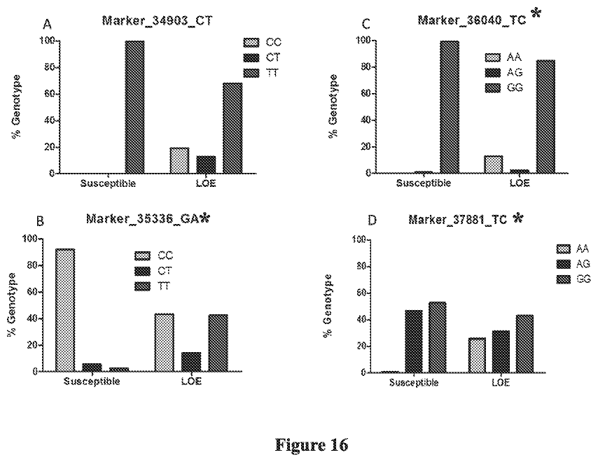

FIG. 16 illustrates the genotype frequencies for the SNP within Marker 34903 (SEQ ID NO: 61), Marker 35336 (SEQ ID NO: 62), Marker 36040 (SEQ ID NO: 63), and Marker 37881 (SEQ ID NO: 64).

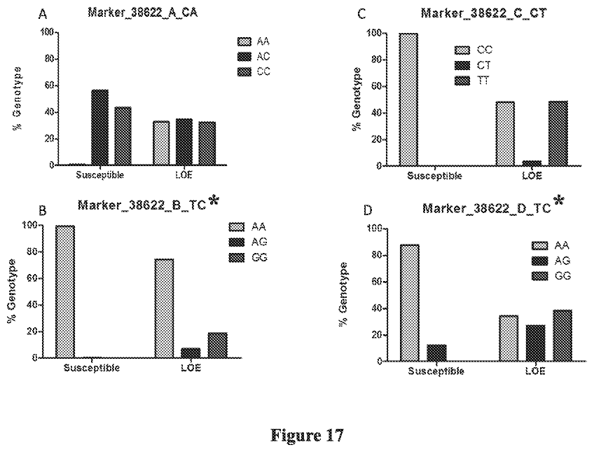

FIG. 17 illustrates the genotype frequencies for the SNP within Marker 38662_A (SEQ ID NO: 65), Marker 38662_B (SEQ ID NO: 66), Marker 38622_C (SEQ ID NO: 67), and Marker 38622_D (SEQ ID NO: 68).

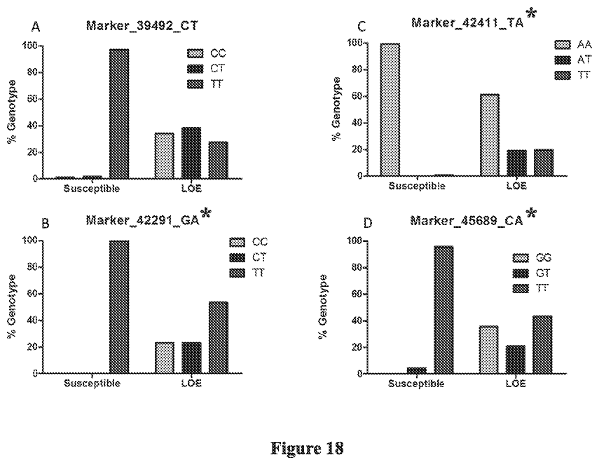

FIG. 18 illustrates the genotype frequencies for the SNP within Marker 39492 (SEQ ID NO: 69), Marker 42291 (SEQ ID NO: 70), Marker 42411 (SEQ ID NO: 71), and Marker 45689 (SEQ ID NO: 72).

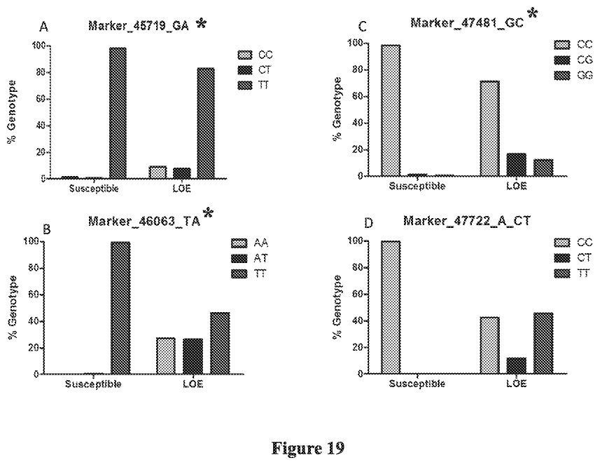

FIG. 19 illustrates the genotype frequencies for the SNP within Marker 45719 (SEQ ID NO: 73), Marker 46063 (SEQ ID NO: 74), Marker 47481 (SEQ ID NO: 75), and Marker 47722_A (SEQ ID NO: 76).

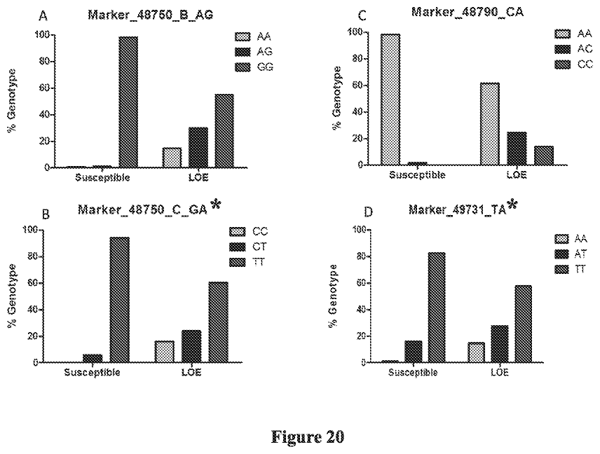

FIG. 20 illustrates the genotype frequencies for the SNP within Marker 48750_B (SEQ ID NO: 77), Marker 48750_C (SEQ ID NO: 78), Marker 48790 (SEQ ID NO: 79), and Marker 49731 (SEQ ID NO: 80).

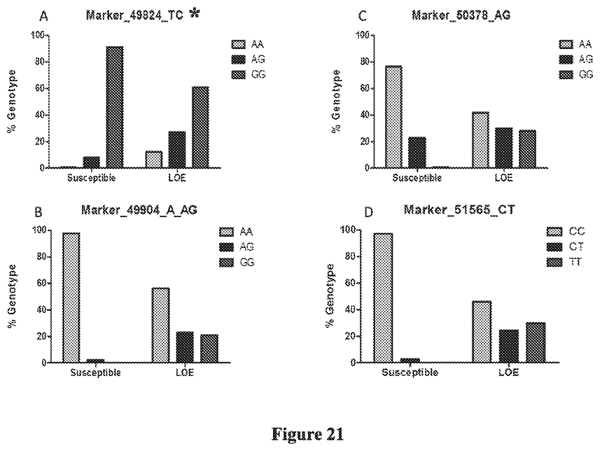

FIG. 21 illustrates the genotype frequencies for the SNP within Marker 49824 (SEQ ID NO: 81), Marker 49904_A (SEQ ID NO: 82), Marker 50378 (SEQ ID NO: 83), and Marker 51565 (SEQ ID NO: 84).

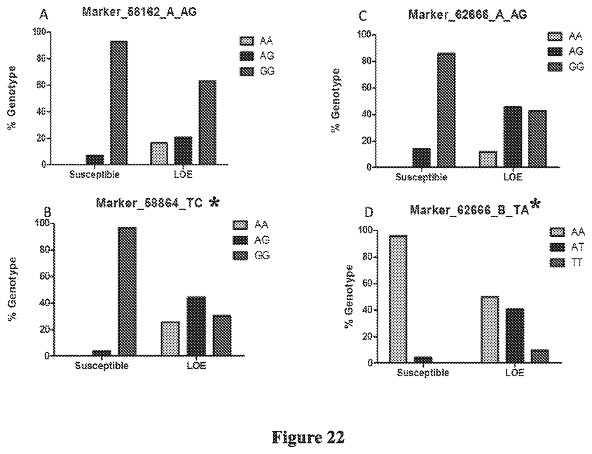

FIG. 22 illustrates the genotype frequencies for the SNP within Marker 58162_A (SEQ ID NO: 85), Marker 58864 (SEQ ID NO: 86), Marker 62666_A (SEQ ID NO: 87), and Marker 62666_B (SEQ ID NO: 88).

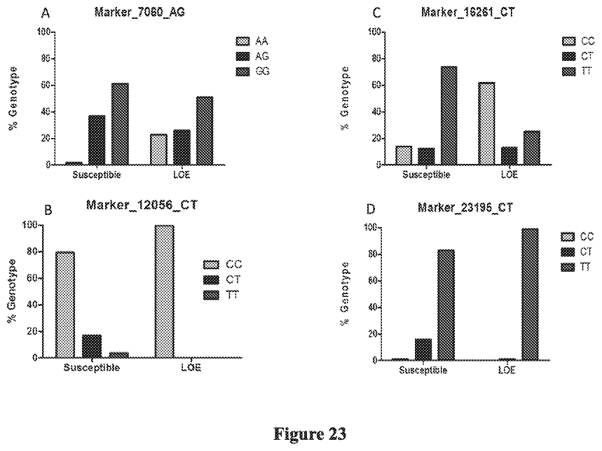

FIG. 23 illustrates the genotype frequencies for the SNP within Marker 7060 (SEQ ID NO: 89), Marker 12056 (SEQ ID NO: 90), Marker 16261 (SEQ ID NO: 91), and Marker 23195 (SEQ ID NO: 92).

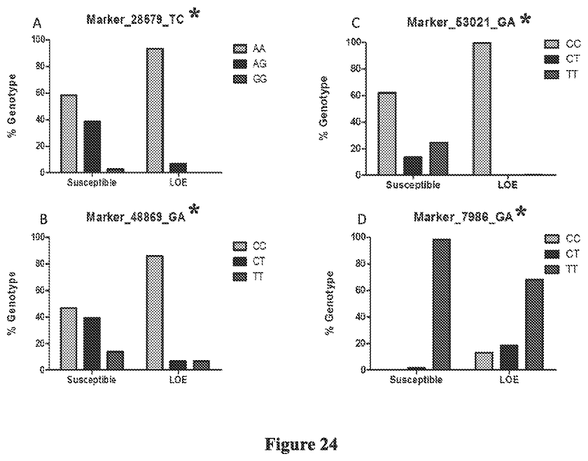

FIG. 24 illustrates the genotype frequencies for the SNP within Marker 28579 (SEQ ID NO: 93), Marker 48869 (SEQ ID NO: 94), Marker 53021 (SEQ ID NO: 95), and Marker 7986 (SEQ ID NO: 96).

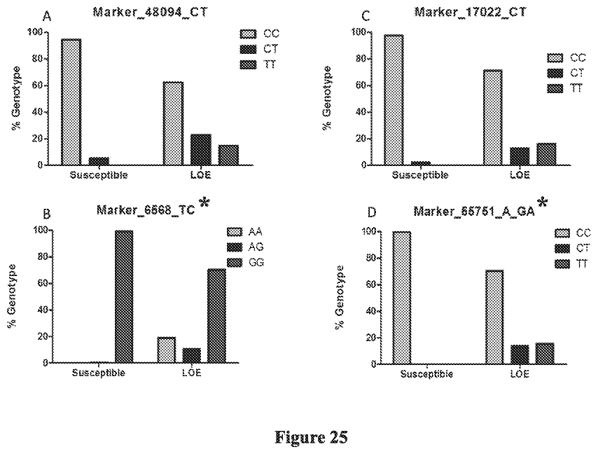

FIG. 25 illustrates the genotype frequencies for the SNP within Marker 48094 (SEQ ID NO: 97), Marker 6568 (SEQ ID NO: 98), Marker 17022 (SEQ ID NO: 99), and Marker 55751_A (SEQ ID NO: 100).

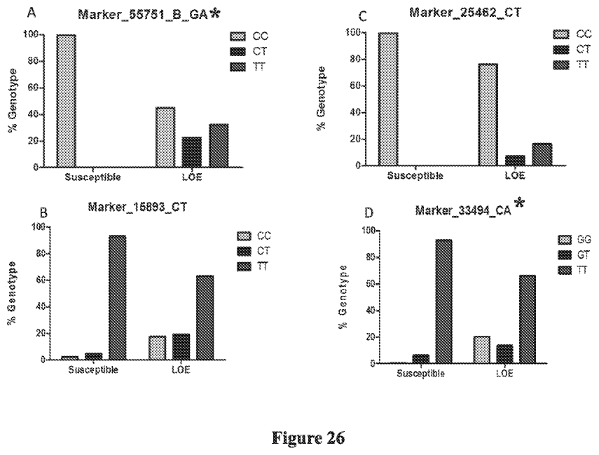

FIG. 26 illustrates the genotype frequencies for the SNP within Marker 55751_B (SEQ ID NO: 101), Marker 15893 (SEQ ID NO: 102), Marker 25462 (SEQ ID NO: 103), and Marker 33494 (SEQ ID NO: 104).

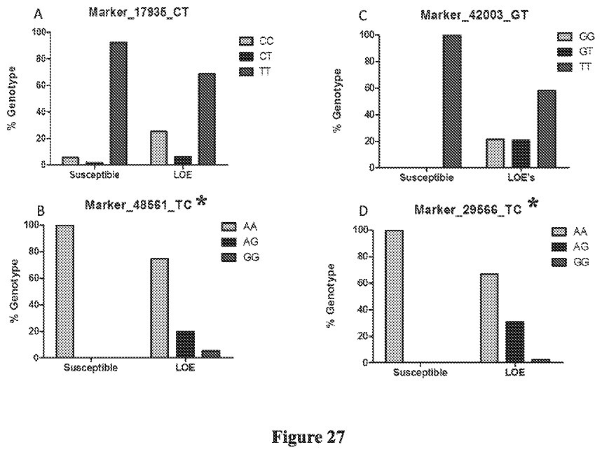

FIG. 27 illustrates the genotype frequencies for the SNP within Marker 17935 (SEQ ID NO: 105), Marker 48561 (SEQ ID NO: 106), Marker 42003 (SEQ ID NO: 107), and Marker 29566 (SEQ ID NO: 108).

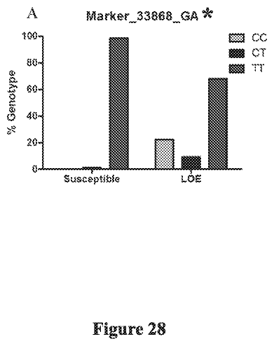

FIG. 28 illustrates the genotype frequencies for the SNP within Marker 33868 (SEQ ID NO: 109).

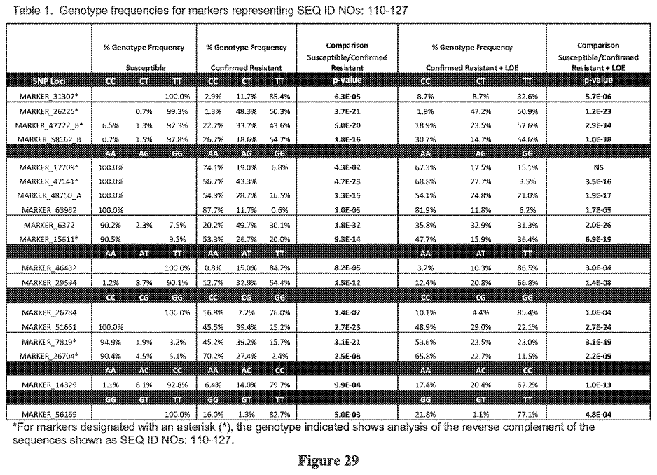

FIG. 29 presents Table 1 which displays genotype frequencies for markers representing SEQ ID NOs: 110-127.

### DETAILED DESCRIPTION OF THE INVENTION

### Definitions

Herein, “macrocyclic lactones” or “MLs” means products, or chemical derivatives thereof of soil microorganisms that belong to the genus Streptomyces including, but not necessarily limited to, avermectins and milbemycins. These molecules are used to treat species of endo- and ectoparasites in a wide range of hosts. Avermectins in use include, without limitation, ivermectin, abamectin, doramectin, eprinomectin and selamectin. Available milbemycins include, without limitation, milbemycin oxime and moxidectin. Macrocyclic lactones have a potent, broad antiparasitic spectrum at low dose levels. They are active against many immature nematodes (including hypobiotic larvae) and arthropods. A single therapeutic dose may persist in concentrations sufficient to be effective against incumbent nematode infections for prolonged periods after treatment.

Macrocyclic lactone (ML) heartworm preventatives were developed for the treatment of dogs and cats, which were not already infected, to prevent establishment of adult infections by targeting the developing L3/L4 stages. Macrocyclic lactones also have effects on the microfilarial stage (L1). Macrocyclic lactone endectocides such as ivermectin (IVM), milbemycin oxime (MO), moxidectin (MOX) and selamectin (SLM) are used during the transmission season for chemoprophylaxis for heartworm in dogs and cats.

Herein, “responsiveness” means that a nematode responds following exposure to a macrocyclic lactone (ML). In embodiments of the invention, a nematode may respond by being sensitive or resistant to a ML. Sensitivity or sensitive to a ML means that the macrocyclic lactone adversely affects the exposed D. immitis nematode. For example, a ML may be lethal or sub-lethal to the D. immitis nematode, shorten its life-span or inhibit its ability to reproduce. Resistance is the reduction in effectiveness of a drug, herein MLs, in curing a disease or improving symptoms (e.g., eradicating heartworm organisms from a dog). AD. immitis nematode may be ML resistant if the drug meant to neutralize it is ineffective, less effective or has reduced effectiveness. AD. immitis nematode may also be ML resistant if the drug, at a specific dose that is meant to neutralize it, has reduced effect. In embodiments of the invention, responsiveness of a nematode to a macrocyclic lactone may be determined in vivo or in vitro.

Herein, “loss of efficacy” or “LOE” means that there is at least a perceived decrease in responsiveness of nematodes to MLs. The perceived decrease in responsiveness may be perceived or may be actual. In one example, the decrease in responsiveness of nematodes to MLs may be real, in which case the nematodes may be said to be resistant to MLs. In another example, the decrease in responsiveness of nematodes to MLs may be perceived and not real. For example, in the case where a dog infected with heartworm is treated with MLs, for the purpose of eliminating heartworm from the dog, the dog owner may not be compliant in properly administering the MLs to the dog. In such a case, the heartworm infection may not be eliminated from the dog because sufficient doses of MLs were not administered, for example. The dog owner, or other observer, may mistakenly believe that MLs were compliantly administered to the dog (e.g., the owner believes s/he administered MLs as directed but, in reality, missed administrations, administered inadequate dosages, etc.) and, because the heartworms were not eliminated from the dog, the heartworm parasites are resistant to MLs. In at least some of these cases, heartworms are not eliminated from the dog because of the lack of compliance. In these cases, continued presence of heartworm may not be due to ML resistance of the heartworm organisms (i.e., the decrease in responsiveness of the heartworm parasites is perceived and not real). In cases of LOE, generally there is no confirmation that the heartworm infection is actually resistant to MLs.

Herein, “resistant” or “confirmed resistant” generally means that the heartworm organisms were shown to have at least reduced responsiveness to MLs. In one example, dogs infected with heartworm are treated with MLs, using a regime known to normally rid dogs of heartworm infection (i.e., compliance of the ML treatment is not in question), but the treatment does not rid the dog of heartworm organisms. Such heartworm organisms, which would normally be eliminated from the dogs by the compliant treatment, are not eliminated because of their reduced responsiveness to ML. Such heartworm organisms are said to be resistant to the MLs.

In one example, a D. immitis nematode may be said to be resistant to a ML if less than about 93%, less than about 91%, less than about 89%, less than about 87%, less than about 85%, less than about 83%, less than about 81%, less than about 79%, less than about 77%, less than about 75%, less than about 73%, less than about 71%, less than about 69%, less than about 67%, less than about 65%, less than about, 63%, less than about 61%, less than about 59%, less than about 57%, less than about 55%, less than about 53%, less than about 51%, less than about 49%, less than about 47%, less than about 45%, less than about 43%, less than about 41%, less than about 39%, less than about 37%, less than about 35%, less than about 33%, less than about 31%, less than about 29%, less than about 27%, less than about 25%, less than about 23%, less than about 21%, less than about 19%, less than about 17%, less than about 15%, less than about 13%, less than about 11%, less than about 9%, less than about 7%, less than about 5%, less than about 3%, less than about 1% or if 0% of nematodes died following exposure to a LD95 (a lethal dose or concentration of a drug that should have killed 95% of D. immitis nematodes) dose or concentration of a macrocyclic lactone.

In another embodiment, a D. immitis nematode may be said to be sensitive to a macrocyclic lactone if at most about 5%, at most about 4%, at most about 3%, at most about 2%, at most about 1% or if 0% of nematodes survived following exposure to a LD95 (a lethal dose or concentration of a drug that should have killed 95% of D. immitis nematodes) dose or concentration of a macrocyclic lactone.

Herein, “nucleic acid”, “nucleotide sequence” or “nucleic acid molecule” may refer to a polymer of DNA and/or RNA which may be single or double stranded and optionally containing synthetic, non-natural or altered nucleotide bases capable of incorporation into DNA or RNA polymers. “Nucleic acids”, “nucleic acid sequences” or “nucleic acid molecules” may encompass genes, cDNA, DNA (e.g. genomic DNA) and RNA encoded by a gene. Nucleic acids or nucleic acid sequences may comprise at least 3, at least 10, at least 100, at least 1000, at least 5000, or at least 10000 nucleotides or base pairs.

“Nucleic acids”, “nucleic acid sequences” or “nucleic acid molecules” may be modified by any chemical and/or biological means known in the art including, but not limited to, reaction with any known chemicals such as alkylating agents, browning sugars, etc.; conjugation to a linking group (e.g. PEG); methylation; oxidation; ionizing radiation; or the action of chemical carcinogens. Such nucleic acid modifications may occur during synthesis or processing or following treatment with chemical reagents known in the art.

Herein, an “isolated nucleic acid molecule” may refer to a nucleic acid molecule that does not occur in nature as part of a larger polynucleotide sequence; and/or may be substantially free from any other nucleic acid molecules or other contaminants that are found in its natural environment. As used herein, an “isolated nucleic acid molecule” may also encompass recombinantly or synthetically produced nucleic acid molecules.

Herein, the term “identity” or “identical” refers to sequence similarity between two or more polynucleotide molecules, at one position in within molecules, or at more than one position within the molecules. Identity can be determined by comparing each position in the aligned sequences. A degree of identity between nucleic acid sequences is a function of the number of identical or matching nucleic acids at positions shared by the sequences, for example, over a specified region. Optimal alignment of sequences for comparisons of identity may be conducted using a variety of algorithms, as are known in the art. In one example, sequence identity may be determined using the well-known and publicly available BLAST algorithm (e.g. BLASTn and BLASTp). In another embodiment, the person skilled in the art can readily and properly align any given sequence and deduce sequence identity/homology by mere visual inspection.

Herein, “single nucleotide polymorphisms” or “SNPs” refer to genetic variations (or non-identity) at specific locations in a genome (i.e., polymorphic site). Generally, at a specific position in a genome, the identity of a nucleotide may be invariant or constant. At some positions in a genome, however, the identity of a nucleotide may not be invariant. At such positions, there may be a nucleotide present at the position at a relative higher frequency than other nucleotides, when the genomes of different individuals within a population are analyzed. The nucleotide most commonly found at such a position may be referred to as the wild-type nucleotide at this position. However, there may be one or more other nucleotides found at this position at relatively lower frequencies. These nucleotides may be referred to as alternative nucleotides. The frequencies by which the alternative nucleotides are found may vary. In one example, the SNPs described herein may play a role in responsiveness of nematodes to MLs. In one example, the SNPs may identify or tag a region of a genome that may play a role in responsiveness of nematodes to MLs (i.e., the SNP itself is not directly involved in the altered responsiveness to MLs but may be genetically linked to genetic changes that are involved in altered responsiveness). In one example, presence of one or more of the disclosed SNPs may indicate that the parasite whose genome contains the one or more SNPs is less responsive to MLs compared to parasites that do not have the SNPs.

As used herein, the term “polymorphic site” may refer to a region/specific location in a nucleic acid at which two or more alternative nucleotide sequences are observed in a significant number of nucleic acid samples from a population of individuals. A polymorphic site that is one nucleotide in length may be referred to herein as a “single nucleotide polymorphism” or a “SNP.”

Herein, “marker” or “markers” generally refer to nucleic acid sequences that can contain one or more SNPs. These nucleic acid sequences can be of different lengths.

Herein, “genotype” refers to the genetic constitution of a cell, an organism, or an individual (i.e. the specific allele makeup of the individual) usually with reference to a specific character under consideration. In the context of this application, genotype generally refers to identity of nucleotides at positions of SNPs. In one example, aGG genotype may mean that at a specific position of a gene (e.g., a polymorphic site) which has two alleles, the nucleotide at the same location in each allele is G (guanine). Alleles are alternative DNA sequences at the same physical locus, which may or may not directly result in different phenotypic traits, but generally within the context of this application, correlate with decreased responsiveness of parasites to MLs. In any particular diploid organism, with two copies of each chromosome, the genotype for each gene comprises the pair of alleles present at that locus, which are the same in homozygotes and different in heterozygotes.

Suitable approaches for use in determining genotype are known in the art and may include, without limitation, PCR, RT PCR, qRT PCR, SSCP and hybridization with allele specific oligonucleotides. Other approaches may include nucleic acid hybridization to DNA microarrays or beads, restriction fragment length polymorphism (RFLP), terminal restriction fragment length polymorphism (t-RFLP), amplified fragment length polymorphism (AFLP), and multiplex ligation-dependent probe amplification (MLPA).

Herein, “consists essentially of” or “consisting essentially of” means that the nucleic acid sequence may include one or more nucleotide bases, including within the sequence or at one or both ends of the sequence, but that the additional nucleotide bases do not materially affect the function of the nucleic acid sequence.

### Genomes and SNPs

In one aspect, the invention relates to isolated nucleic acid molecules possessing at least 80% sequence identity to SEQ ID NOs: 1-127, over their entire length, and comprising the alternative nucleotides at the location of the SNP (i.e., polymorphic site), the alternative nucleotides indicated by the underlined nucleotide in SEQ ID NOs: 1-127, as disclosed in this application. The alternative nucleotides generally have a lower frequency of occurrence at the indicated positions within the sequences, as shown in FIGS. 1-29. In one embodiment of the invention, the genome of a nematode parasite, or a population of parasites, may contain one or more of the alternative nucleotides at the polymorphic sites shown in SEQ ID NOs: 1-127. The presence of these alternative nucleotides generally correlates with reduced sensitivity of the parasites to MLs as compared to parasites that do not contain the alternative nucleotides.

In another aspect, the invention relates to isolated nucleic acid molecules comprising, consisting of, or consisting essentially of the sequence depicted in SEQ ID NOs: 1-127.

A nucleic acid molecule of the invention may comprise a sequence corresponding to that of SEQ ID NOs: 1-127 over their length, and containing the alternative nucleotide at the SNP site (i.e., polymorphic site). In embodiments of the invention, the nucleic acid sequence may be at least about 80%, at least about 81%, at least about 82%, at least about 83%, at least about 84%, at least about 85%, at least about 86%, at least about 87%, at least about 88%, at least about 89%, at least about 90%, at least about 91%, at least about 91%, at least about 92%, at least about 93%, at least about 94%, at least about 95%, at least about 96%, at least about 97%, at least about 98%, at least about 99% or 100% identical to SEQ ID NOs: 1-127, but that was isolated from a nematode having the alternative nucleotide at the position in each sequence shown by the underlined nucleotide as disclosed in this application.

In other embodiments, the nucleic acid molecule of the invention may comprise a part of, or fragment of, SEQ ID NOs: 1-127 that also contains the polymorphic site and the alternative nucleotide at the polymorphic site. In various examples, the fragment of SEQ ID NOs: 1-127 may be 5, 20, 15, 20, 25, 30, 35, 40, 45, 50, 60, 70, 80, 90, 100, 120, 140, 160, 180, 200, 220, 240, 260, 280, 300 or more nucleotides in length.

A nucleic acid molecule of the invention may be derived from a D. immitis nematode containing one or more of SEQ ID NOs: 1-127 as disclosed in this application. As used herein, “derived from” may refer to a nucleic acid molecule that was isolated from a natural source, e.g. a Dirofilaria immitis nematode. It may also refer to a nucleic acid molecule that is man-made, e.g. recombinantly or synthesized on the basis of a nucleic acid molecule isolated from a D. immitis nematode.

### Detection of SNPs

SNPs may be detected by any method that can determine the identity of a nucleotide at a specific position in a genome (e.g., polymorphic site) and that allows for comparison of the identities of nucleotides at the specific genome position between individuals or populations of individuals. Differences in the identities of nucleotides at a specific position may be indicative of a SNP.

A variety of methods may be used to detect SNPs. In one example, hybridization-based methods can be used. Hybridization-based methods generally rely on hybridizing complementary DNA probes to the site containing the SNP. In one method, dynamic allele-specific hybridization (DASH) relies on differences in melting temperatures resulting from mismatched base pairing. By designing probes that differentially hybridize based on nucleotide changes in target genomes, SNPs can be detected.

In one example of a hybridization-based method, molecular beacons can be used. Molecular beacons are single-stranded nucleotide probes, with a fluorochrome at one end and a fluorochrome quenching molecule at the other end, that can form a stem-loop structure and place the fluorochrome and quenching molecule in close proximity to one another. In absence of hybridization of a molecular beacon to a genome region, the fluorochrome will be quenched, due to its close proximity to the quenching molecule. When the molecular beacon hybridizes to a genome region, the fluorochrome generally will not form a stem-loop structure. Under these conditions, the fluorochrome will fluoresce, due to the increased distance to the fluorochrome from the quenching molecule.

In one example of a hybridization-based method, oligonucleotide microarrays, which are high-density arrays containing hundreds of thousands of probes, are used for hybridization to SNPs. By comparing differential hybridization to redundant probes, it is possible to detect SNPs.

In one example of detecting SNPs, enzyme-based methods may be used. In one example of an enzyme-based method for detecting SNPs, restriction endonucleases are used to digest a genomic DNA. By determining the fragment lengths that result from the digest, it can be determined whether certain sites within a genome fail to be cleaved by the endonuclease due to a nucleotide change (e.g., alternative nucleotide) in the sequence recognized by the endonuclease.

In one example of an enzyme-based method for detecting SNPs, PCR (polymerase chain reaction)-based methods are used. In one example of this, two primer pairs are designed such that only one of them will function to amplify a site containing a SNP, depending on whether the SNP is present. The sizes of the amplified products are distinguishable, therefore informing which primer pair functions, and whether the SNP is present.

In one example of an enzyme-based method for detecting SNPs, nucleotide probes are designed to hybridize to a genomic site and produce a mismatch, whether or not a SNP is present at the specific genomic site. An endonuclease (e.g., Flap endonuclease) that cleaves one of the probes, depending on whether a mismatch exists, is used. Using fluorochromes and quenching molecules, attached to one or more of the probes, SNPs can be detected.

In one example of an enzyme-based method for detecting SNPs, primer extension is used. In this method, primers are hybridized to genome DNA immediately upstream of the SNP. DNA polymerase is then used to extend the primer in a mini-sequencing reaction. The sequencing reaction determines the presence of a SNP.

In one example of an enzyme-based method for detecting SNPs, the 5′-nuclease activity of Taq DNA polymerase is used. A TAQMAN™ assay is performed concurrently with a PCR reaction. The method is set up so the PCR reaction will extend through a site containing a SNP, and release a fluorochrome from a probe hybridizing to the SNP region, depending on whether the probe contains a mismatch due to presence of the SNP.

In one example of an enzyme-based method for detecting SNPs, DNA ligase is used to ligate two probes, one hybridizing to a SNP site in a genome, depending on whether the SNP is present, and a second probe hybridizing adjacent to the SNP site. If both probes hybridize to the genome without mismatches, ligase will connect the two probes, which can be measured.

Other methods of detecting SNPs exist, including for example, detection of single-stranded conformation polymorphisms, temperature gradient gel electrophoresis to detect duplex mismatches due to SNPs, denaturing high performance liquid chromatography to detect mismatched duplexes, high resolution melting analysis, use of mismatch-binding proteins, and others.

In one example of detecting SNPs, a biological sample comprising a D. immitis nematode may be obtained from a subject. The subject may be, without limitation, a dog, fox, wolf, coyote or cat. In the context of the invention, a biological sample may be any sample (e.g. bodily fluid, excrement, organ, tissue, etc) from a subject. The biological sample may be from a subject that is known to have, or is suspected of having, a D. immitis nematode infection. The D. immitis nematode may be isolated from the biological sample with standard separation methods and techniques.

A nucleic acid sample may be isolated or obtained from a D. immitis nematode prior to use. Methods of isolating nucleic acids from organisms and tissues are known. Such methods may include, but are not limited to, traditional DNA extraction, with proteinase K digestion followed by phenol chloroform extraction, sodium hydroxide extraction, and physical disruption, followed by purification, e.g. by cesium chloride centrifugation or high performance liquid chromatography (HPLC); or the use of commercial kits. A skilled person would appreciate that different approaches may be used to isolate a nucleic acid sample from an adult D. immitis nematode in comparison to a microfilaria. In an embodiment of the invention, the nucleic acid sample comprises genomic DNA.

The nucleic acid sequences of the nucleic acids from the parasite may be determined using any one of numerous methods known in the art. In some techniques, sequences of separate pieces of the genome are assembled into linear whole genome representations of the parasite using computer-based methods. In one example, massive parallel sequencing may be used. Massive parallel sequencing (also called “next-generation sequencing”) may encompass various high-throughput DNA sequencing methods. One such method is the HiSeq2000 system from ILLUMINA®.

Through comparison of sequences from separate parasites or parasite populations (e.g., comparison of a consensus or reference genome obtained from parasites sensitive to MLs with a consensus or reference genome obtained from parasites resistant to MLs), presumptive SNPs can be identified.

The presumptive SNPs can be analyzed further. In one example, high-throughput SNP analysis using multiplex PCR and MALDI-TOF mass spectrometry (SEQUENOM® analysis) was used. Generally, this system uses extension of an oligonucleotide primer or probe using chain terminating nucleotides to product different sized PCR products for each allele of a SNP. The different sized PCR products are analyzed using MALDI-TOF mass spectrometry.

### Disclosed SNPs

In one example, genetic markers from D. immitis include the sequences below (SEQ ID NOs: 1-109), where the underlined nucleotides (i.e., the polymorphic sites) indicate the nucleotide position within the fragment that correlates with resistance to MLs (i.e., the alternative nucleotide). In these sequences, the nucleotide at the underlined position is generally different than the nucleotide found at this position in organisms that are not resistant to MLs (wild-type). In the sequences below, the nucleotide underlined in the indicated sequence is the alternative nucleotide which correlates with resistance to MLs. In the heading for each sequence, the nucleotide change from wild-type to the alternative nucleotide (alternative correlates with ML resistance) at the polymorphic site is shown (e.g., C in wild-type and A in the alternative sequence is designated as C-+A). The genotype frequencies for each SNP at the polymorphic sites are shown in FIGS. 1-28. In FIGS. 1-28, for markers designated with an asterisk (*), the graph presents the genotypes of the reverse complement sequence, as compared to the nucleotide sequence presented in SEQ ID NOs: 1-109.

MARKER 617 (SEQ ID NO: 1): C→A
AACATAAACATATTGAACTGAATCCTGCAAACAGTTCTCTTATAACGTGAACCATAACTAAATTTAGAGAAAATATG
AAAAAGAAAAATAAGTTGCTTTTGCTCGTGCACCAACTCTAATACCCAGGAAATCAAGAAGTGATAATGAGTAATGT
CATCATTAGATTCAGTAATTGGTGACACTATCAATATTATTATTATTATACTTAAAAATACGACGACCACTTATCGT
AACTTAAAGCATGCATAATACGACTGTCATCATATTACATTTCTTCAAGTTCGTATTGGACAAGTGATT
MARKER 714 (SEQ ID NO: 2): C→T
GACAAGCGTTGACGGGAGAGACGATATAATAATAAAGAAGGCATTGGGTATCAGAAGGCACAATCCAATTATAAATG
CCAAGGCAAAATGAATAAAATTTATGCTGACGATTTGATCAATTACGAAGAATTTCCGATCGGCTCGAATCTTTGTT
TGTATGTGCACTACTGTTAACTTAATCTTTGTTTTATATACTTTTGCGTGTCATATATAATATATTCATGTCAACTG
ATACGTTATGATGTTTTTTTGTAAATTAAGTTGATCGGAAACCTGAAGTCTATTTCAAATTTAAGAAAT
MARKER 814 (SEQ ID NO: 3): T→C
TTTTAGGAAAATGGTGACTGTAGAGAGATATTATCGGAACGACAAGGTCCACTTCGAACGGGTCTTTTATTGTCGAC
GGATTGTGAACCAAGTTTTGGCATTCATAATGACAGGTAGCTATTTTTCCATCATCCCATTTTTGTATTAGTGCAAG
CAAGTCATGAGTCGAAAGAAAATCTCAAAAGAAAAAAATGAAATTTCAGGTTCAAAGGACTGCGTCCATTATTCGCA
CTGGTTGATGAGAACGTACAGATTCCAGAGCGGCAATGCTGCACAGTATCTTTTGTTTCACTTCTGAAT
MARKER 887 (SEQ ID NO: 4): C→T
TCGATTAAAAATTATCATCGATAAAATTCTAAAATTTATTTTAGTAAAATTATTATTATTTTGATGAATAAGTTAAC
AAAAAAATTTTAATAACTTTTTGATTCGCCAAAAATCTAATTCGTTAAAAAGTCGTTCCAAACAGATATCGCTTGTT
CGATGAAAATGTCCGGTTGTTAGAAAATCATAAATTGGTTCAAATAATTTTCCAGAACGTTCGAAAAAATATTCCCT
TGTATCGGATAAATAACCATTACAATTTTCCACTCGTGTTGCATGTGTTTCTCGACAAAAATCAGCTAA
MARKER 1514 (SEQ ID NO: 5): T→C
TCAACAGAAATCGAGATTCCAAAAAGTTTCCTACAAATACTTAATTATCAATGGATATTTAGTTTTGTTATCTGTTA
TCATAAGTTCTGCTTCTTACACGATTAAAAATGTCCAAGAATTTTTTACTATTCAAATGAGGGAAATAAAAAACCAA
TGCCAATAATATCCAGAAACTACATACATCTTTCTTTTTTCGAAGCTCATCTATTCCGGCCGAAAACAATGAAGAAC
ATTAAAATTCTTAAAAGATAGTCTTAGCCTTTTCCTTGACCACTATCTTAACTGTCAGCGCTAAAATGT
MARKER 2557 (SEQ ID NO: 6): T→C
AATAGTCGTCTCATTACTTTTTGACTTTTATAATTCGAGAATCTTATGTAGTCCTTCACTTTACCCTTCTTCTGTCG
AACTAAGAATTACAGCATTATTTTCGAATTTAATGTGTAAAAGACAATAGCAGATTTTGTAATTTTGTGTTAACCTC
ACTTTATATTTCGCTTCATATCGTGACAGAGAATTACTATTTCAGAGAGTATTACTTGTCACCAGAGAATCTCCAGA
AAGATTTTTATTTACGTCGGAAAATGGACAAAAATGGTTTCTTATCATTAGCACTGATAGCTAGTTTCC
MARKER 3367 (SEQ ID NO: 7): G→A
TATCTCTTGTTGTGTGTTCTGCATTGTATCAAAGTGGGTAAATTTTGCTTTAGACGTTGACTTATTGTCTTTTTTAA
GTTATATTCTAGTCCATGTTTTTCTCTTTGCAAATATTTTTTTCCGCCGCCTATGATTCATTGTTTTGTTTGTAACT
CTCTATTAAGTTGCTTTTAGTTTGAATTGTATCAAAATTTCAAACATTTAAAATACGCACTAGCACTATTTTTTCTT
ATCTCAATTAAGCGAATCCCGGAACAAGATTTAATCGATTTCCGAATCACAATTAAATCACTGGAAAAC
MARKER 3488 (SEQ ID NO: 8): T→C
ATTTTCCTTAACAAATCATTTTCAAACGAAAAAACATTAAAAAGTGTTAAAATAAAATGGTGATATTGATAAGAAAT
TAATTCAACCTGCATATCAATTCTTGTAGCGGCCATTTTCTTAGCAAGTTCTATAGCAGCTCGATCCATATCACCTT
CTTGCTCTAATGTCAATTCCGGTTCCGGAATTTTTTTTATTTTGCCATTCTTCATCTTTTTTTTATTTTTTACTGAT
ATAGCTATAGACCCTTTCTCCCGTGCATGCCTGTAGGCCTGTTCTGATATACAGGCTTGTGAACCACTG
MARKER 4553 (SEQ ID NO: 9): C→T
TTCTGGGGTAGTTATACGGAAAATTAGACAATGAAGAGAATCAAAAAACATGCGATTTTCAAACAGAGGAACTTTGG
TACTTTTGCCTCGACTTACTTTATTTTAAAACCCATACAAAATAAATGTTTCATTTGATTGATATTGTCGTACTAAT
AATTAGAGCTTCAACATTAGGATTTTAATAACCTTCAATTTATTTCAGAATTTAAGAAACTTACGTATGGATGGAGA
AAATATAAAGAATGGCGATGACAAATAAGATTTGCTATGAAAAAACTAATGCCACAAGATCCGAATGCA
MARKER 5266 (SEQ ID NO: 10): C→T
TTTATGAACAAAAATAATAAAAATTAGGATAACAGATATCAATTTCTTTTAGCTATAAATATACGCTTCGATTGAAA
AAAGCTTTCAAATTATAATTAAGGCATACGTTACGATATAGACAATTAAGTCGACATTAATTATTTGAAATATTTTA
AATTTTTTTCTCTTTCTTTTTTTCTATTCTCTTCCAAAGTGTCAAATAGTTATGAAATTGTCAGAAGCTAAAATGAT
AATATTATTCAAGTTTATTACCTAATCTTTTATCACCTCATTTCTTATCATTTATCTGAAAATCTAATC
MARKER 5365 (SEQ ID NO: 11): G→A
ATGTTGAATTTTTAATGAAACTTTTTCGGTGCATAAGCATTACAGATCTGTAAGCTGTGCAAACCCTGTTTCTTTGT
AAATTGAAACAAAGATCATTTATTGTTTCCAGCGTCGATTTGACCTGGATAAATGTGGTACCAAAAGTAGATGACGA
GAGGTAAGTGCAAACAAAATGCACAAAAATGATTTTGATGCACTCAAATCATTTTTAAGTTTTGTGCAATTTTCCAT
TTTATAGTTTCGTGATCGGTTGTTATTCATCAACTTGATTTTGTTTGTTTTTTGTGACTTATATTTCAT
MARKER 5667 (SEQ ID NO: 12): G→A
TTTGACACTTTCAGATACCTTACAAACTCATCTCCAGCACCCAATTTACAATATCGCTGCCTAAATAAAGAATTTAT
TCGGATATGAGACTGTAGTTTTCATTCCGTACCAATCATAGTAGAACAGATCTATAGCATGGTGTCCTACTAAAGTT
GTGACTGGCTATTAAGTATGTGGGTGTTTTTACGTGTGCGTGGGTGTTTGTGCGTGTGTGCGTGTGCGTTTCTGCAC
ATATTTTCGTGCGCGGTGTCTGTGTGTGTCCGTTTGTATATGCCGAGTGTAGCTGTGTGTATGTTCTTG
MARKER 6568 A (SEQ ID NO: 13): G→C
CACTCATAATATACCTGTCAACAAACTCAGAAATCTGAATAAAATGACGCAAAAATGACAAAAACATTTTATCAACC
TTTTCTTCATCACTCCCCCGCATTTCCAATTTTCTTCCAAACTGTTTTTGTCGTGCTACAAAGTCATCAGCCACTTC
ATTTTCTTCAAGATGGTTCGAGACGCCATTCTTGGATTCACCCCTTATTTCAACTGTTTCCGAAGTCCCAGCAGTTG
AAGCTGAACCTAGCATTTATATCACCACCCGATGTCAAAAAATGACAGCGGTCAGAGAATACGACTTCC
MARKER 6568 B (SEQ ID NO: 14): G→A
GCTAGGTCAACAGTTGGTTTATTTGGACTTATACGATATTAAACATAATATCGCCTCATATACACAGAAATATCAAA
AAAACGAACACAGCTAAATCGAAGAATACGAACAAATGTTTTAAAAATTATATTAAATCTTTTAATGCTCTCTACAA
TGTCGTATCTTCCCTTTTGTCTGTATTTCTCCTTTCGTTCCACCACTGCTATTTCTCATGCCTTTGAACTATGGTTC
TCGTTGCGTCGAATTGTCCTCGAAACTGTTGTTTCTGTCGAATTACGTCGAACTGCTGGACTTTGTCGG
MARKER 7633 (SEQ ID NO: 15): T→C
ATATCTCACTTCTGACATAAATTGAAGTGGCACTGATTTGAATGAAATGATAAATAAAATAAAGACGACAAGGTAGT
GGAAAAAAAAAGAGGAGAAAACACCGTTTAGTTTTGGATGCAAGCTCGAATCTGAGTTTTCTTGCAAACCGTACACT
GATCAATTTTCTTACACAAACATAAGAAAAAAAGAAGTGATTTTACTGTAGCTGTATCGTATAATTCAAATCATATA
TATATATGTTTCAATAATCTATACATTTATGTATATTTTTTTTTGAATGGAACAGTGAATGATTTTAAA
MARKER 9400 (SEQ ID NO: 16): T→C
ACAAATGCCATCGGGAGAGAAATATCGTTGGCGTACTGATCACATTGGCGGTATCACTTCTTTGAAAACTCCAGCTG
GTATTGTGTATCATTTCATGCAATACGCTATTTTTGATCGAATATGTCGACGGCGTAGTGTTTCATTTTCCAACGCA
TCTTACGTTGCGTGTATGGATGATGACGGACAATTATTGGAATATCAAACACCGGATCGATTGCATTCCGTAACCTT
GAAACGTGACATATATGGGAGAGTAGTGCAAATAACTTCAGATGGCGAAAATATTTTCTTCGAATATGG
MARKER 9473 (SEQ ID NO: 17): C→G
ATAATATATATTTCCATTGATAATATTTTTCATATTATGTGATGTTTGAAATTTTCTGCAATTGCTACATTCCGATT
AAAAACTTTTATTATCCGTACTGGAGAATTTTGCTTTTTTTTGACGGTTTGTTCAATAAGTTGTCAATATATTGTCT
GCCTTAGTAAAACCTTTCTAATCTATCCGTTCGAATTGGAAGTTGAAAGTTCAGCATCATTCTTTTAGTGAGGTGTT
TAAGTTGTTCAATAGATATTATTTAGAACGATCTCAATTAAAATCTTCTGAATGATTTTATGTTTTTAT
MARKER 9858 (SEQ ID NO: 18): A→G
GCAGCACATTGCACACAGTAAACTGCAAACTGAATTAAGAGATATTGGGTTGAATTATTTCTAATTTAAAAGGATAT
AATAAATGACTTTGATGATTGTTGATTTTAAGGTATCTCGGAAGACTCCATCAGTCTCAGTGCTCTAGCAATCGCTA
TAGGTACTAAAAGAAAAGAAAAGATGTCTCGTTATTCACTTTGAAATGTACATATCAAATCATTTTGTCGTATGAAA
TTAAGTATATTATGTCTAATCGTATCATTCGAAATGAATTTACTGTCACTGTTAGAACTATTTAGGCAG
MARKER 10349 (SEQ ID NO: 19): A→G
AGAGTTCAATCGCCAAGTTGTTCTTTTTCTCGCTCGCAGAGATCAAAACGGTGTTGGCTATACACTCATTCATCAGG
CTGTGATAGACATCTCTTAGAATTTCAGTGCTTTTCTGGATGAAAACATTATTTCTCAAACATGACACTTAAGGACA
ATAGTGCGTGACTTCTTTGTTAACGTACACGAGAAAACAAAACAGATGATGCTTGTTATCTTGGTGATAAATGTGTA
TTCAGAATAATGTTATATATCTTTGCGTGACAAATATCATTTCGTTATACTTCGGATACGCCTTTTTAT
MARKER 10520 (SEQ ID NO: 20): A→G
AACTTTACTTGAACTTTTTTGGTGTTCAATTTTGAATATTATACCAACCATTCAGAAGACTGTATATAGAAATGAAC
CTTCAAGAATTAATCGAAATTTTTATTAAAATCTTTTATTTGAATATTTCATTATTTAAACTCATTACTATTTGCAG
TATATTATTAGATCTAATGTAGAAAAAAAAATCAGATGGCAAAAATAATATCATAGGTTTGTTTTTAAAATTCATTG
CAAAATTCAGTGCGCCGTTCCAGTCGCTCGTAATTACCCTATCCCTGAGCTTTACAAAAAGAATGCTTT
MARKER 10678 (SEQ ID NO: 21): A→T
AGGTATCTAGATAGCATAATAAATTACTACACAAACCGATGGAAACGCAAGTTTGGCGTTGCGTGTTGATACAAAAT
ATTAGAGCCAAGGATGGTATCACATGTAAAACTGCAATTTTGCTATTTGTTTAAAGCAAATAAGAAATAAATATTTC
GTTCTTATTCTTTAATTTATTTCATCAGATGGCTTTGTTATACCATAATTGTAAATCTGTCATATCTTAATTGCGCA
ATAGCCCAAGATTCTTGTATATTCTTACATTTCACAATTTATTTTCTTATTTCTAGTTTTAGAATTATA
MARKER 11676 (SEQ ID NO: 22): A→G
AATAGCTACTCACAGCTTAAGTTAACTAATGGATTCTTGAATTTATTTAAGCGTGTAGTTAAGCGATTAATATGATG
GATGCCCAGAATCGCTTTGTCTTATAGTTTTGTCTCGACAGAAAGGATGCATTGTTGTCTTGAATTTGTTCAAGGGA
AAATTAAATAGGTTTCTTTCAATGACTCCTATTAAATTTTTTTGAATTTAGGCTTGCATTGCGTGTTCTGATCCACT
ATTAGCACGTACGGGTATCGCAGTGCCATGTGATGCAGCACTATGCAAAAACCACCTCCATGTCACTTG
MARKER 11933 A (SEQ ID NO: 23): A→G
TCTGTTGTAAGTTTCACAATCCAGTTAATTTAAGCTCAGCTTATTTGAAATTTTCAACAAAATTACGAAAATTACTT
TCTCGGTTCATTTTTTTCAACCACCAAATATTTAGCATAATTGGCCTGAAATCGTCAAAGTTTACAAACTTTTGTTC
AGCAATCTTCTCTTACTCTTACAATAAACATGATTAACTTGTCGTCATACCAATCTCGTTTATAGCAAATTCTTTTC
AAAAAAACATTGCTACAAATTTTATATCGCATCATTTCAACACGCATAATTATTTTTCATATATGAAAA
MARKER 11933 B (SEQ ID NO: 24): T→C
TTCACAATCCAGTTAATTTAAGCTCAGCTTATTTGAAATTTTCAACAAAATTACGAAAATTACTTTCTCGGTTCATT
TTTTTCAACCACCAAATATTTAGCATAATTGGCCTGAAATCGTCAAAGTTTACAAACTTTTATTCAGCAATCTCCTC
TTACTCTTACAATAAACATGATTAACTTGTCGTCATACCAATCTCGTTTATAGCAAATTCTTTTCAAAAAAACATTG
CTACAAATTTTATATCGCATCATTTCAACACGCATAATTATTTTTCATATATGAAAAACCATATTATAA
MARKER 12716 (SEQ ID NO: 25): A→G
ATTAACTCTGAACCCAAAGACTGTTGGTTAAAATAAAGATCTATTTTAGTTATACATCTAACATTAAAGGTTTTCGT
ACGGAAACAAGTAGGTTTGATAATTTTCATGTAACTGTAAAGAACACCTGTGAAAGGGATCAGTAAAATTTGGGGGA
TGTAGCACGGAAATATGAAGCTGAGTGTTTTGTACCCAAAAGTTTTTCAAATCTGCGAAATAACGAGAGGTGTAATG
ATCGTTTTTAACCAAATTTTTTGATTCTAATCCTTCCCACAGTTTTGAAATTCAGTAAGCATTTCTTTT
MARKER 12925 (SEQ ID NO: 26): T→C
TTGCAACAAATCAATAATAAAAGACTTGCGGCTAACAATATATTTGATTCTTTTTTACCGTTATTATTATGACAGGT
AATAATAGTATTACAAGCATATTTGTAGGTGTCAATTTTTTCAATTCAAATTTTCTTAATTCATTATTTCTTCCTTT
CCTTAATAAATAGTCTTTCCATTTAAGAATTAACTTTTTGAAATCTTTAATGAGAAGACACAAAAGATTCCGGATAA
TTTTGCATCATCTTTTCTATTTCGCGTTAGTATTTTATGTTTTCAACAGATTTTTATGATTTAACTATA
MARKER 13063 (SEQ ID NO: 27): C→T
GATAAAATGGGTTCTTGTCAAGCTCATTTGGCATATCTTCGTCTTCTATATTTATATCCTTTAATATCTTCTCTTTT
TTCAAATTTTCCTTCCCGACGTTTTCCATATCGACCTCTTTCTTCATAAATTTATCTTCCTCATTTGCCTCATTTTT
TGACTTTTCATCCGTTTCATCCTTATTTTTCTTTTTTTCATCTCCTATTTTACCTTTTCCTTTATCAACTTCTATCT
TAACTTTCTCAATGTTTTTTTTATTTTCTTTCATCTTTTTGTTTTCTTCTATTGACATACTATAACAAA
MARKER 15000 A (SEQ ID NO: 28): T→A
TTTTACGAACAATTATTTCATAAAAGATTCGTATTTTTGATTAGTTTTTAAGAATTTTTTTTTATTATTTTTAGCCA
ACAAATATATTTTTCAAAATTGTTAAATTTGAAATTATAAATTTCAACTAAAAAAAAGCAAAAAGCTAAGCCAATAG
AAATAACATACATGTGTAATATAAAATATAAAGTATTCGAAATGAAAATCAAAGTTTCATAACAAAAAACAAAAAAT
ATTCTAACCTTTTAGATTTCATCAAAACTTCACTAAAAAGTTAAATTTAAATTTTCAAATTGTTATACA
MARKER 15000 B (SEQ ID NO: 29): A→G
CGAACAATTATTTCATAAAAGATTCGTATTTTTGATTAGTTTTTAAGAATTTTTTTTTATTATTTTTAGCCAACAAA
TATATTTTTCAAAATTGTTAAATTTGAAATTATAAATTTCAACTAAAAAAAAGCAAAAAGCTAAGCCATTAGAGATA
ACATACATGTGTAATATAAAATATAAAGTATTCGAAATGAAAATCAAAGTTTCATAACAAAAAACAAAAAATATTCT
AACCTTTTAGATTTCATCAAAACTTCACTAAAAAGTTAAATTTAAATTTTCAAATTGTTATACAATGAT
MARKER 15709 A (SEQ ID NO: 30): T→C
TCAAAGACAAAATGAAGAACTTAACAAAAAAAAGGCCAATAAATAAAGGCTATTTCGTGAAAAATCTAAAAAAAAAA
AGATCTGTTCCTTTCGAATCAAGTGATTCTTCCTACTACATTCGTGTTGTAATTCTTACTTGTATACAGTCCCCAGT
TTTTCGACGATAAAAAACATTTCGATAAGTGAGTTTGAATTAATTGAATTTTAAAAGATCATAAAAATAAAATCAAA
ATAAAAAGACCAAAATTAAGTCTGATAATTCCAGAAAACACAATAATAAATATACAAATAATAAAAACT
MARKER 15709 B (SEQ ID NO: 31): T→A
AAATAATTCACTAATTTCTCATCATCAAATTATTTCGTACAATCGATAAATCAACGATTATAATAGCGAAGAGAATG
AAAATTAATGTGGTGCACAGTATACGGACCCCATATACAATGTTCAACAGAGATGAACATTTTTTTTCTATTAAAGT
TTTCTGTTCGGCGAAAGAAAGACACTTTCTAACGATGCTTTCCTCCCAACTCCCCTTGCAATGATAGAGGATGCAGC
CAAGATTCGTCGACTCAAGCAGCATCACTCAACCGGCCATCACTTCGGGACCTTTTTCCCTGCCTTTTA
MARKER 17333 (SEQ ID NO: 32): A→G
CATTGCGAATGACCGCTATGGAATATCAATTAGCAGATATTAATCGTGAATTAAGCACATTGGTGGAATTTTTACGA
CCAAATCGAATTTCAAAAAATGCTACACTTGCAACATCAGCAACCATTGCAACATATAACAGTACTTCGATGCGTAA
TGTAAAAAAGAAATGTAATGCATCTGAAAGCTGAAAATTCATCTGATATATTGAAGCAAAAGGTAAGATTATTTTTA
AGATATCATTCTTGATGCTCTCATAATTTCTACATCAAATTTAATCAAACGATTCATTTATGTTCATTT
MARKER 18110 (SEQ ID NO: 33): C→T
TTCTTGTTGTACCTATCATAGATGATAACTTAAGTACCAATAGCAATAGTGCAACGATGCAAGGATTCTGATTAATG
ATTATAAAAGTTTAACCAATCTTCTTCATTCCTTCTAATCAAGAGAAAAAAAAATGAGAACATTTTTATGACATTTG
AAGAAAGGCAATTTATCGCTGAAAATTCTACTGCGATATGGAAGTATCAGATAGAGAAAATAAATATTAAAATATGG
ATTTCATACGAAAAATGATAAAAGATAATAATTTACATTTTGGTGCTTTACTGATATGATTGGAGTATT
MARKER 19999 (SEQ ID NO: 34): T→A
CGATATTTTTTGGACGAATCAAACCTTTTTGGGAAATCATTTGATGTCACAAGCATGGTTTGAGAAATTTTTTTCCG
AATTAGTTCTGCTAAAAATACTCCAAATGAGTCTAGTGGAATTAAGCTAAGCACCTTAAGTAAGTTGAGAAAAACGT
TTCCATTTGACTAACAAGGCTAGTATATCGACATGAGACAGAAATGGTTATTACTTCACTCACTTCATGAAGCGAAT
ACGAAATATCTGTTCACTTTAGTTTCAATCTACTATTTTACCAATAAACGTGTTCTTTTCCGGATAAAT
MARKER 20570 (SEQ ID NO: 35): T→C
TCTTAATTGATTTTCTTAACTCGAAACACTTGTCTTGATTACTGTGCTGTACTTTATCTTATTAAATTAAATAATTT
CCATGACCACTTCATACCATTGACCATCAAACTTTGATGAAGTTTATGTGTGAAGTGCCAAACAATCATTCATCCCT
TCAGTTTAACTTATTGCTGGTCAAATTCATAAAAATGCAAATTATCAAGCAGATAGTAATTCAGTGAACGTAGCGTA
TTCTCGAAATTTCTTTCCTTGTATTTACCTTATATAGAACAACGTATATTTGTAGCATATATTCAATAT
MARKER 20587 (SEQ ID NO: 36): G→A
TTTCTGAGTTTGCGTTACAGCGCCAAATCTTCACGGAGATAGATAAAATACTTATCGTGAAATTTTGGCGCCATGAT
TTAAAAAACACGGAGATAAAAATAAAATGCTTATCGGTGATAATTTAGCGCCATAATATGAATGAATTGAAAAAACA
ATTTGAGTAGAAACATGACATAGAGTTTTCGTTTTCTGGCTACGAAAATGGATGAATTTTTCTGGAATCGAATTCAG
TCAAAGAAATAGGAACGTTGTTACTAAATGATCGAAAAGCTTTCTAAAATTAAATTTATGACGTCTAAG
MARKER 20698 (SEQ ID NO: 37): T→C
ATCTAAATCTTCGTTTTATAGTGGTAAGACTTCCATTTGCTGCATTCTTGCAAATTAAGCTGTTGAAAATACTTTTT
TTTTTGATAGATTTCCAATTTAATCATATTATAAGAAGAATTAATTTCGAATAGAATTTTTAAATCATTTAAACTTT
AAGTTTTAAAACTAATATAAGTTATGCAGATTTCGCGAAAAAGTCTCATTTGTTAATTCAATTATTCCAAAATGTAA
TAATTTTATAAATTCAAATTTAAACTACTACTAACTTCTGAAGTCAGGAGCCAGTAGCAACAACGTAAT
MARKER 21554 (SEQ ID NO: 38): A→G
AACTTTACATTTATATTCAATTTTTTTTTATTTTGTTTGTTTTTAGAAATTTGAAAATGGGTACTAATCAGTGTCAT
TTGCAGCCTCTTAGACCCTCTTTATAACGACCGATTCGATGAAATACGTCATCAATATGCCAGTTTATTGTTCGGGT
GGAGAATGTTTTCAAAAGTTGCTGAAGTGATGAAGTATAGTGAGAATGCACCTTATTCAGCACCATTAAGAAGTAAA
TTTTTGCTTTGGAATTTGACAAAGACAAAGCAGGAAGTTGACAACGATGTTCTGATGAAACGGTTTCGA
MARKER 22174 (SEQ ID NO: 39): A→C
GTCTATTTTGGCTGTCTTCTAATAATTCATTTTGTAACCTTTTGAAATATGATAAATGTAGAAATTTTTTCTTCCTG
GTCTATAATAGTTTAATAATGTGTTGTAGTAATAGTTTTGGTGCCGTTGAAATATTTCAATGATATGCTATCGCAAA
ATTAGGAATTCAAATCAAGGTTACAAGATAATTCAAAAACAAACAACGTAAAAATGAAATAATTTCTTCTTCTTACT
TACCAACAGGCATATCATCATCATCCTCAAATTCATGACTATATTTAACATTGTCATATTTGAATAATC
MARKER 22254 (SEQ ID NO: 40): C→A
CGACGCAAAAATCTTTCAAATTGTCACCCAGTTCTCTAAGTGATTCCAATGATGTTGGTAAACATTCTGCATGATGT
ACCGGGTAATGAACTACCAAGTTGTTTTTTGCTTTTAATACAACTCGCAAAGATTCTGAAAACCATGAAATTAAGAA
AGATTAAAATAATCTGAACTCTTTTTTTCATTTTTCCTTGAACTTAGCAATATACTGAGTTGGATAAAATTTAGAAA
CGAAATTTCGCAAATTTATTCAGTAAATTCAGGAAAACTCGGTTTCGGTATTCTAAATATAAATAGATA
MARKER 22259 (SEQ ID NO: 41): A→G
GTTTCTTTGGTTTATCTCAGTAAGATTTGGGCGGAAATTTCAGTTATACTTTTCATTTCCATGTGCTGTTTTAAATT
TCTTCCATATTAGTATAATTTTCAAATAATTGTAGCGTCACTGGTTTATTTAAGGATAACAGGTTGGACTGCAGTGG
CTGAGAAGTGTCTTGCCGGTCAATTGTTTGTTGGTGATCAACTTGTACGAGTTACTGATATCGACATATATAATACA
CGGCAAATTCCATTCGTTTTCAGTACTGCATCAAAAACGGGATTATCGGTACTTTGTAAATCGCAGTAT
MARKER 24708 (SEQ ID NO: 42): C→T
GACCCCTGCTCACAAGGCAGTTCCCACAGACAATCACACATCTAATCACACACATCAACTCATCCGACGTAGGCTAT
CAATAAGGAAAATTGCATTGCTTTATCGTCTAACTGTAATAAACATCTACATAATGAAATTATTTCGCCACTATGAC
AACTAATATCGCCCAATGCAAATATTTGTCTCAGAGTTATTCCCTTTTAACAGCTGTTGAACGAATAGATAGGACGT
CATGTGGATGATCTACTTGTTTCAAAGGTTGAGGTAACACATGAAACACATGAAAACGGTAATTTAAAA
MARKER 25276 A (SEQ ID NO: 43): A→G
AAAGAATGGTCAGCAAGATGTGGAAAATCGATTACTATAGTTGAAGTATGAATCGAAGAGGTTTTTTTAAATTCTAA
GAGAACGAATAATCGGCAAAGAGAAAGTTGAGTAACCTTATTTTGCCTTGTTTTCAGTCAATTTATAATATGCGGTT
AATTGTGTTAAAGAAAGTACAAGGTATGAAATCTAAGCCAAGAAATAAGAGAAAACAGCTAATGATTATTTCTGCAT
TTTTTCTTTTTCGACACAAACTTGGAACCAGAATCAATTGAACTAGTAATCAGATTTTGATTATTGCTT
MARKER 25443 (SEQ ID NO: 44): T→C
TTAGATTTTGCTGAAGCATTGTTGGTTAGATCGATGAAAATATAATTATGAGAGATTTTGTTGAAATTCAGCAACAA
AATTATTATTCATGTCTTCATGCTGTCAGTTTTGTTTTTATTTCTTCTTTGACATCGGTTATATTTTTGTCTTCCAA
CAATATAAAAAAAAAATTATAATCAATTGGTAATCAAATTAAAACTCTAATTGTTAGCTCCCTAAATCAGCTTTAAA
AAAATAATTGCTTAATTGGTATTTGCTACTATTAGCAAACTGAAACTATCCTTTTCTCGAATGGTGAAC
MARKER 26447 (SEQ ID NO: 45): G→A
ATGAGCTGATATTTGATATGCATATTAAAAATAGGGTAAATTACATTAAGTTAGATATCGTTCGGATAAATTAATTA
GAAAAAATGTTTACCAATTAGATCGCAATGATGTAAAATTTCACGTATTTTTATTCTTAAGATTTATTTGCAAAATT
CAAAAATATGTCTTATGAAAAATAATATTTCTGTGTAAGAACAAGGGACCGATTCACTTGATTTATTCGCAAACAAT
CGAAATTCAAAATTAGTAATTTTAAATATTGCTTTATTCAAACCATACCAATAATAATTTGAGAGATTT
MARKER 26730 (SEQ ID NO: 46): A→G
ATTGATTGATTCAAATAAGAAATTTAAATTATTTCCCCTTTTTTTCAAAAGATTTAACAAATATTATTTATTTGATC
TCCTCGTTCGTTCTTATCTTTTTGATTATCAATCCATCCTCCTCCATCATATAGCTAATTTATTTTTTGCATCGTAA
ATCAATTGATGTATGATTGATTTCTTGATTATAAAAAGTTAGAAGAATTGAATTGCTTAAATTTAATTATTGATAAT
GAAATATTATTATATTTCAAAATGATACGAAGAAATATGACGATGATAAGAGAAAATATGATATTTATC
MARKER 26974 (SEQ ID NO: 47): C→T
TACGATAAGTTATTTTATTTTACACATCTCCATCCTTGACTAGTGTCCGTGCCGACTGTCGGACTTGAACCGACAAC
CTACTAATTACAAGTCAGTTGCTCTACCCAATTGAGCTAAGCCGGCCATCTAGAATGTGCGACCCCGTCGTGGTACA
TCTTCTATAATCGTTTGGTATTCAGGACTCTCTTCTTTCGTGGGTGGAGGATCTTGATACAGTTGACTATTAAAAAT
AGGGCCTTTGTTAGTCTGTTACAACTCATAGACAAAGGCGACAATTTTAGCTTACATCTTACGTTATGC
MARKER 27080 A (SEQ ID NO: 48): A→G
ATGGTAGAAAATTATATGAAAAAATATCATACTAAAAATATAACAGATTGTTATAAGGTATGGTTTAAGAATTTACA
ACAATTGATTATTTATGATAAAAAAAAAAAAAGTAAATCAGTGAATCATTAAGATAGTTATGATAAGCAGTTTGTAT
TCGGTAAAGCGAATGATTAGAGGAATTATGGGACGAAACGTCTATAACCTATTCTCAAACTTTTAATGAGTATGACG
TGTCTTGCTTGCTTAAAATTATTTCAATGATCATTTCACTTTACCAGTATGATCATGATTAGACTTGAA
MARKER 27349 (SEQ ID NO: 49): T→A
TTAGTATCGATATTATCACAAATGATATCACTTTCATCAATACTGGATACGATTTTATTAGTATCATAATTTTGTGG
CTCGCATTCCGAAAGTTTTACACGTAGAAGATTAACCTGCAATATGATTTATTTTATCATTTTCGAATATCCAACTT
TGAAATAATTCGAAAATGTTGAAAAATTTTGAAAAATTGTTAACAAAATATTACAAAAATATCAAATGAAATTAAAT
AACTGTCCATTTCAAAAAAAGAAGAAAAATTATGAAATTACCAATTAAAAACAGGACTTATTAATTAAA
MARKER 27461 (SEQ ID NO: 50): G→T
TGTGGAAATAAAGTACAATTAATTGCTGTTCGCTTAATAATATTATTTTCATTCTTGGCTTTTTTTTTCTTTCCCCG
TGATATTATAAAATATAGTTTTTTAATTTTAACAAATCGTCATAATTATTTAAAAAATACTGAGGTGAGTAAATGTA
ATTGGTTGCTGGAAAAAAAGTGGGTGATGAGAGGTGAATGAAAGCAGAATAGTTTATGATTGCATCAAATTTCCTCC
TTAATCTGTGATTAAAATCAAACAAAACCCGAAAAGTTTCTTCTTCGCCTTTTTCTTCTCTTTGTTTCA
MARKER 29128 (SEQ ID NO: 51): T→C
CGAAATCCGCCGCGTGCATTACTTTGCGCTTGTTGATTACGACGCATTTGTTCGTCGTTGATAACCTTATCAATCAT
CATACGTCCGTTACGTATGCAATCAACATCGCCAGTTAGGCTGAAATCAAATGGATGGCGATGATATCAAAAACAAA
AATAAGGAGTATTTGCTGAATCATTTCTTTTTCTGTATTATTATCAAAATTTTCTCCTTTCCATTGTTTCCTTCTTA
ATCAAGTGAATGCTCATTTCATTTTGAAATAATCCAACGTAATAATTCCCCATATTCCCAATTACTTTC
MARKER 29168 (SEQ ID NO: 52): A→G
AGAAATATTAAACTTTGAAAAGATGTGACATGTTCTGTAACAAAAGCCCAAAATTTCGACTGCTGCGGCTTGAAGTA
AAATTTTGGAATATGCTACATCAGTAGTGCAACAGATGGTTCGATAAATAGTGGTAAGTGATGGGAATCCTAGGAAT
AGATGGGAATTGTATTTCAGATATAAATTTGATGCATATTTTCATAGTTGATTATATCTACGATCACACGTTGAATA
TTCTAAAAGCAAACTGTAATTAACTAATTGAATTTGAAAATTTCCAAGAATTAAAATTGGTAACAAAAA
MARKER 29455 (SEQ ID NO: 53): T→A
ATTGTCAGGAATGAGAAGCAAGTTTTGGATACTTAAGGGATGAATGGAACACATACATGGCAGAAAATGTTAGTAAT
CAAACCATTTAAATTACTTAGCCACTATGCTAAACTTTCTAGAAGTATGGTTGAACGTTTAAAAACCTTCGCAAAAA
TTGTATTAGATTATCTTAATCTTCCCTACATCAAAACAGAGAATTTTTGTTCTACGACGTGAGTCTGCATGTATTAA
GGAAGTTCGTATCATGACGTAAATATCCTGAGTGATTATTGAATTCAGAAAATGAGCTTTTTCATTTGG
MARKER 29816 (SEQ ID NO: 54): G→A
ATATGAGTGTTACATGTGTACGTTACATGTAAATATTATATGTTATATGTAAAAATGTCATGTATAGCATCTATTCA
CGTGTACGTACACGTGTATATACATATACATTGATACTTAATACGTATACGCATGAATGAACAGATATTATATATTT
ACGTACACTAGACTCACATGTACCTCTGTATACGCATACATGTACAGATATATGTTTGACATACGTAAATTCATATA
TGCTTTTATTTATGCTTATATTAATTGTCACATACATGCCTTATATTTTCGTTGTTATAAACACATAAA
MARKER 30575 (SEQ ID NO: 55): T→C
GAAAATAAAATTAGCTGAAAATATATGCGAGGTAAAGCACACAGAAGAATTAACTTAAGGTAATATATTGTAAGAAT
TTTTATATTCGGCGCACCTAATAATTTTTAGACCGCATATGCCCAGTATTTGAAACTGGTAGCGCTGTTCGTACTTG
CTGTTGTCCATGTTATGTATATGATACCATTCCTAAATACTTTTGCGGCTGTGGTTTCCAGTGTTGATGTGACTGGT
ATGATGCCTAACACTGGATCCTTCCATCTGCGGCATTTTGTTGAAATTCTTATTGATGTGAGCTGTTTA
MARKER 30991 (SEQ ID NO: 56): A→G
CAACTGTGAATCATAAACATTACTTAAATTAATGAAGCTAGTTAACGACAAATATATTTTTTTATGTATCAGTGCTA
TCATATAACATAAAAACTTACTTTCATTAATAAATGAGCTCAAATATTGACTTTTGTCCAAAATGCTCAAAATGTCG
TCATAATATTTGAAATGAAGATAATTTCACGCTTTTCGAAGCCTCCTCTCACGTCTTTTAATCTTCTTTTCTTCTTC
TTGCTCTAATGGTTCTGCGAAAAACCACGGTGCAATAATCACTTTCCATAATTTATACAGTACATAAGC
MARKER 31796 (SEQ ID NO: 57): A→G
CTGCTTAACTCTTTTCATTTTTCAGAGAATCTTCTCTAAAATTGTGAATTGATCCAAACCAAAGAATATGGATAATG
TGATTCGAATTCCTGGAATTTAGATTTTGAGAGTTTTGAAGTTTTTAAAGAGATTGAATTTCTGTGACCTTCTGGTA
TATTTGATGTCATTTCGGGATGCGTATTTTTGCCGAAAATTTTTGGCCTCACTGCAATCTTGTTAAAAGTCAAAAAA
ATTCAATCGTAGAATTTCGGGTTTACCTGATATTACTGGAAATCTCTGATCTTTGTTCTAGATTGCTGT
MARKER 32164 (SEQ ID NO: 58): A→T
ATAAAGAATTTGCAACTCTGTATACCTTTTTGCAGTGCAAAAGCGGATGAATTCTTCACTGCAGTGTGACAGATTCC
TTTGATAAAATTGCTTCGTTCTTATGTAAACTTGGAAATTCTCGGTAGTTATGCTTTTGCTAGTTGAAAATGTTCTG
CTCTTGTAAAACATGCAAAAAGAGATTATCTTTGTTCTATTATGGAAAGATTCTTTTGAAATTTTGACGACTGAGAA
GACAAATTTTATCCCAACTTGTCATCTGCAATAAAAATTTTTCCTGACCTGTTTCTTAACCTTCCAAGT
MARKER 32223 (SEQ ID NO: 59): T→C
AAAATCAAATCAATATGATCAGATAACTCATACTTATCTTACTGAAAATTCCTCATTCAAGGGAAATAAATAATTGC
AATTCTTGATTCCGATCATGGATGATTTTCAAGCAAATTACCAATGATATCTATCGATAACGATTACAGCATACAGC
TATAACTTATTATTGATTGAATTGATGAAAATAATTTTACCAGAAATTTATCAATGTTTATCTCATTGCAGTATACG
ATGTTTAGTGTGACAACACTTTTTCTTGGAATAATTGTGCATAAATCATTGATTGCATTTAGTATTGGA
MARKER 34439 (SEQ ID NO: 60): T→C
TCCTGCCCACATTCTTTCTACTTTAGATAATCAACAGGAGTTAGTTGAAAGAGAAGACTAGGAACAGTTGCAACTTC
TGAATCTTTCTGACTTTCTTTCGTTTTGTAAATTATTTATTTGTATAAATTTAAAATTCGAAGAGAAATAATCCAAG
GTCCAACTTCTTTTTCTGTTAGTTCTTGCGAATGCTCCATCAAAATGCAAAAATATGATTAGAATTCTGATGGAAAT
TAACAAAATCGATTAGATAAGAAAAGTACAAAACAGAAACTAACTTTTTCTCCCATTTTCATATTATAG
MARKER 34903 (SEQ ID NO: 61): T→C
TCATTGCTTTAATACTTTTTAACGAGAATTTTCTCGATCAAAATAAGATCTGCAATTGATATACGTCAATAAGCGAA
CATTAGCTGTATTACACGCTAATATTCACATATGATGAACGTTGTAAGCGTCATACATCAACATATATCCATCCGAT
AAATAATGACCACTACACATTGCTACCAACCATCCTATCCCGCCACTATTTGAAATGAACTGAGAAGGAGTTATCGA
CACAGGCTTCCTAGCAACCAAACAAAAGACGAGACAGATGAATAGATAGACAGACAGACGAACATACAA
MARKER 35336 (SEQ ID NO: 62): A→G
AGATTCTGGTTATTATTGTATTTCTGATTTATTTAATCCCAACTTAAAGATTCATTGGCTATTGTTTAGCATCTATA
TCAATTTTATAAATAAATAGTAATACCTGATGAAAAGCAATAAATAATTAGATGCAAATTTTAATTAGATACAGTTT
GATGGAAAACATTGAAGCCATGTACAACTAATTTATGCATGTTGAATTATGCATGCATAATTAATTTATGCATGACA
GCAAGTTTGGTATAAAATTAATTTTGTATGAAGATAAAATTTTATAAATAATGATAATAATGCTGGTAA
MARKER 36040 (SEQ ID NO: 63): T→C
ATTATTGAAAAGAATAATGTAGCTAATTAGTTGAAGCTGTTAAAAGTAAAGCTAAAAAGATGATGGAAATTATTCGT
ATAAACATTCTTTGTAAACAAACAGTCATTTCTGTGAATAAACAATTATAATTATAAACAATACTTTTCAAGACAAT
AAAAAAATTAGGAAGCATTGTTGTGATAATCAATAGTTGATAGACTGTCAATGTATTTTTATCAGTCGTGCTGCTTT
TTTTCCCTTTCTTGACTCATTTATTTTATTATTTATTGATAGAATGTCAATATTCTAGTCATTTGTTAT
MARKER 37881 (SEQ ID NO: 64): T→C
ATCTTAACTTGCTTTAAACAAATAAATTAAAACAGCCCAATGTTCCAAGAAAAAAAGATAAGTTAAAAGTGGGGTGT
CCAAAAATTTATGAATTGAATTGGACAGTTATTCAGATCCTGAAAATACGCTTCTCTGATCACTGCAAATATTCCCG
ATAAATAAGTGAACATTAGGTTAATCTTAATTTTCCCTTAACTTTCCTTAGCCTTTTTTAAATTTTTGGATTATTCA
AGCATTTTTATTGCGGTATCGTTTTTGTAAAAAAAAAAGTATAATTCAACATTCAGGCTCGACGTTATG
MARKER 38622 A (SEQ ID NO: 65): C→A
AATTAATAAAAAGAAAGGAATACGATAAAATATCTATTTTTTGAAACTAATCAAACATATTCCTCACTGCTCACCGG
ATAGTTGCTTTCTAATTTTACATTAAGAAATATATTTTTTTTTTTCAATAAGGAAAGTTATGCAGACTAGGAGAATT
CTACTCTGAAGAAGAGATAAGCATGTTAGAATTATTAAAATCTATGGAAATATCCTTAAAAGAATGCCTATAGTAGC
TCTGATTTCGAAAAAAAAAGCAAAAAACAAAATAACAAATTCTGCTCAATTGAAATAAAAAACTTTCCT
MARKER 38622 B (SEQ ID NO: 66): C→T
TAAAATATCTATTTTTTGAAACTAATCAAACATATTCCTCACTGCTCACCGGATAGTTGCTTTCTAATTTTACATTA
AGAAATATATTTTTTTTTTTCAATAAGGAAAGTTATGCAGACTAGGAGCATTCTACTCTGAAGAAGAGATAAGTATG
TTAGAATTATTAAAATCTATGGAAATATCCTTAAAAGAATGCCTATAGTAGCTCTGATTTCGAAAAAAAAAGCAAAA
AACAAAATAACAAATTCTGCTCAATTGAAATAAAAAACTTTCCTTCAACTTCCAGCATCACTGCTGTGA
MARKER 38622 C (SEQ ID NO: 67): C→T
AACTGCTAAAAAATTGAAACTAGTGTTAGATTGATAAGTGGGCAGATTAAAACCAATTGTGTTATTGGCCCGTTAAT
TAGTGACTCTGAATAGCTATGGCGAATCGTATAGTGTTGTACCGACGACGTATCTATCAAATGTCTGCCTTGTTAAA
TTTCGATGATAGTTTATGTGCCTATTATAGTTGTAACGAGTAACGGAGAATAAGGTTTCGACTCCGGAGAGGGAGCC
TGAGTTGCCACATTCAAGGAAGGAAGCAGTCGCGAAGATTACCCACTCTTAGAATGAGGAAAGAGTGAC
MARKER 38622 D (SEQ ID NO: 68): C→T
GAAAACTAAGAAGTAAGTGAAATTTCTAAGTTCTTTCCCAGAAAGGTTAGATCCAATATTTGTTTTCATTTTAGCAT
TTTTATCCAATGAAAAATGTGCCCAATAAATACTTGTATATAGTATTGCATTTAAAAACTTCAGAAAGCACAATGAG
ATCTAAGCTCAGAAATATGACGAATACCAATCCTTTTCCTAGTCTTACCGCTTCTTAACTTTTGTGTCGCTTTATAA
AAATTAAAAATAAAAAGTTGAACAATGGGAATTACATCATTTTCATCTGAATGGTTTATTTCCTATTCT
MARKER 39492 (SEQ ID NO: 69): T→C
CTTCCCTAGCTATGCCTTTTCGTCACTTAAGCTTCNNNNNNNNNNTCTAGCTACGTATCGTTATCATTTATGCTTCT
TTAGCTACGTTTCTCCATCATTTATGCTTCCTAAGCTACGTATCTTCATCACTTACGCTTCCCTAGCTATGTCCTTT
CGTCACTTAAGCTTCTTTGGCTGCGTGTCTTCATCATTAATCTTCTTTAGCTACGTATCGTTATCATTTACGCTTCC
TTAGCTACGTCTTTCCATCATTTATGCTTCCCAAGCTACGTATTTTCATCATTTATGCTTCCTTAGATA
MARKER 42291 (SEQ ID NO: 70): G→A
GATCTTAAAATTCTATGAAACTTCTTCTGCATGGTATTGTTTCCAACAGAATATAATGACAATAGCAACAGTATTGG
TTATATAAAAATATTGACTGCAGCAGGATTATATTTCAAGTTCTTTTAATTTCATTTATTTATTCTTTCATTTACTT
TTACTGTTTTTATGTTTTTCTTCTTTAAAAAATATGATTTCTCTCACTGTTCTCTTTCATCTATCTATATTTATTTG
ATAATTGCTTATATGATAACTAGCTAAAGGGAAATAAACTTTCAGTCATCATAGCTTCATTTTAGTAAA
MARKER 42411 (SEQ ID NO: 71): A→T
CTATACTAATCAGTCCACTATCCATTTTTAGGTTGCAAAAGTTGCAATGACGGTTTGATTTCATCCTCCAATGCAAT
TTTGAGTCTCAATCTCGAGAGATAGATCGATCGCTTTTAGCTTGATTTAGCTTGGTTAATGTTGTGAGGGATATTGG
GCAGAAATTCTGTCAAGCGTTACTTAATGAAATAGTAAATGATCACTGATATTTATTGTTAATGATACTTGAGCTCT
CTAGATTATGAACTGGAAGGTTTTCGATAGAAATAATCGATACATATATTAGAATCGACTTCTTTTTTC
MARKER 45689 (SEQ ID NO: 72): A→C
TCATCTTTTTCACATTTCATTTAATCATCATTTTATCAATTCCTATTTTTAAACAAATTCTTTTCAAATATTCTCTC
TTTCCTTCTCTTTTTGTTTTCCGCTTATTCATTCTAATGATGAACAGATGTAGAAAATTTGCATTCTATTGCTCACT
ACAATTTTGAGTAGAATATATTTAATTATTTGATTCGAGACAGATGGTTATAGCCTTTAGCTTCAGCTTCTCGTTCA
AATTAAGTACTTGTGACCTTTCCAAGTACCATTAAAGCTTTCCTGCGTTTCCTAATTAGAAAAAAAAGG
MARKER 45719 (SEQ ID NO: 73): G→A
GCATTTTAAGTTAAAAGTATCACGCTGCATGACACCTCACGTTTGCTATCTCAAATTGAGTAGGTTAGAATCTTTTT
TTGGCTACTATTCAAATATTAATAATAAATTGCTGCAAACAGATTTCACACCGGAAAAAAATTAAATTTTTCTAGCA
ATGTTTTAACTCCCTTATTAAATATTTATAGAAAATCGACTACTTAAAAAGAATTGACTAACATTTCTGAATCTCTG
CAGAGATTTATAGATGGATTAGCATCCTACAAGTTTTTATCTTTTTGCTATATTTCCATTATTTTTTTA
MARKER 46063 (SEQ ID NO: 74): T→A
GATAAGACGTCTTATTTTGTAATAATTCAAAAATTAATTAATATAGAAGTAAGATCTTGATAATAATTAATATGCTC
AAATTTCTTAATGAGAATATGTTCAGGATGAAGATGAAGTGAAAGAAATTGATAGATTGAGGAAGCAATTGCTAATT
GAAACAGAACAGCTCGTTTCCAATTCTCTTAAAGATTTACTGAAGAAAATTTATTATCCACTTGAAGAAGCTATTGA
TCTCAAAATTCATCAGAAATTAATTCAACAAATTGCTGCCTTGTTGAAGTGTATTAGTATCTTGGATAA
MARKER 47481 (SEQ ID NO: 75): C→G
ACCGCAAAATACCTAAAAATTTCTATAACAACGATTAACACGGCCTCGAACTGGAAGCATATTAATCCATGCGTGGC
TCAAACTTCAATCATAAAGACAAGATCTAGAGATCAACACAAAATGGTGAATTGTTACCCTATCGTTGCTAAAGTTT
GAGAGAAAAAAGTGCTAAATCAAGTAGTACACCAAATTTAGTTAATATTAAGAAATCAATTTAGTACTGAATTTAAA
CAAATGAAATTTTACGATAAAATAAAAAAGTACCTGATCAAACAGCGTCCTCCCGTTATTCCCATTGCT
MARKER 47722 A (SEQ ID NO: 76): C→T
TATAAGACTAGTAAACAGATCGTAATATAATAAATATCGATTTTATTTTAAATTTTCGAAAACTTCCAAATCTATCG
ATATGAAATTAAAGATCAATTTTTAATTTCCATAATATATTTAGATTCTATCCCAACATCACTCATCTTTATGTCAA
CTTATTTAATTCTCTTATTAACATTATATTTCTTGTTTACAATGATAAATTTTATCAATTTTCTAATATGATAGAAC
ATCTTCATCATCTGAAGATATGCTTTTCTCATCTTTGTAACAATTCGTATCGCTTCTGATTTTACTTTC
MARKER 48750 B (SEQ ID NO: 77): G→A
GTTTTATTATTGCTTATTGAATAGTGATAATAACACTTTGATATGATATTGTTTTGTTGCGATCATTGTATTGATTA
TAACCTTAATTAAACGAGGATATTATGGGAAATGTATTTATTACAAAATTAAATATGAAAGGTTGAAGTCTTGACGA
AACTTTCAAACACATTTCTCGAATTTTCTCTGCAAAAATATCGTTACGATTTTTGGAAATTATGAAGTCCAAGAATT
CAATCGAGAGTTCGCCATGTCACTTTGGCTAGTTTCGTTTGTTTTTAATATTTCAATCAAAAGTCAATT
MARKER 48750 C (SEQ ID NO: 78): G→A
CCTTGGATATTGTTCTTGACATCGTTGATCAGAAGGTCACCGTAGTGTTCGGTGAGCGAGATGGAATTGGACTCAGG
TTTATTCTCCGTTTTTTTCATGTTTTTGAATTTTAGAGAGAAAATAATGTTTGTCTGAATGGTTAGCAAACTAATTA
GTTTTTAAGTTATCAGGAACTCGAAGTATCTTCTTTTGCACTTCTTTAACCTTTTTCATCAAATTTTTTAACAGTAA
CAAGATTTTTTTGAGAATTTTCAAAATATTTTTGACTTCTGATGATATTTGATGAGAAAACCATCACTG
MARKER 48790 (SEQ ID NO: 79): A→C
AGAGTATTATTATACATGATGATGATGATGATGATGATGATGATGATGATGATGATATGATGATGATGATGATGATG
ATGATATGATGATGATGATGATAATGATAATGATGATGATGATGATTAATTGCTTATTTTTAATGATTGATAACTTT
AAAAGAAATCATTGAAATTTGATCGAATAAAAATTTTCTTAAAAAAAGCATTTGCTATTTATATAGTAAACCTATAA
AAAATTACTTATTTTTATTACTAATATTCATTTGATTGTATGAAAGAGAAGAGAAAAAAAACCTTTGCA
MARKER 49731 (SEQ ID NO: 80): T→A
TGGTATCACAGCACTGGGTTTAATTTCAACAATCGGTTGACGATCTTTTCGGGATATGCCTATACCCAGAAATGAAC
GTATGCCAAACGATGGTATGTTTGATGCAACAGACGACGTCAACTTAAAATGTGTTTTTTTTTCAAAAATTCAATAT
TTTTAGTTTAAAATTGCACGTCAGTAAAAATTAATTCATAATAAATCTCTTTGATTTCTTCGTTCTCCTTTTTTTTC
AGAAAAAATTGAAATTTTACATACCTGATTTCCAAGAGCATATAAAGCATCACTTAAAGCATTCTGCGA
MARKER 49824 (SEQ ID NO: 81): T→C
TCCTTTTCATGATTTGTAGCTAACCAATAAGATGTGTATATGTTCATATATTTACTCTCCCCTGACTCTTTTACACT
CTCATTCTCTCATTTGTTCATTTAGATAAGTAATATGCGCCTTTCTCTTCCTGATTCTCTCAATCTTTCATCCCTTC
ATCTCCTCAATCTTTCTCCCATTCTCTCAATCTTTCCTGCATTGCATTCATTGATGAAACACGATAGTATTAATAAG
CATAATTTGATAAATTGAAATAATTTTTTTTNNNNNNNNNNTCATTCTCTCAATCTTTCCTGCATTGCA
MARKER 49904 A (SEQ ID NO: 82): A→G
TTTGAATTAACAAAATATTAACAATTACAACTATTTCGGAATTTAATTTAAGAATAATTTAATTAATCAATTTCCTA
TTTTGTATTTTAAAAATTACCACAATAATTATGTAATTTTTGGGATATTTGAAACTTTGAAAAAAGTGGTATTGTAT
TTGAGAATAAATTAATTAATGTAATTCTTGCTGCTCATCGTTCCATAACTTACAAATATTTCTCGGTATTTTATTTG
AGATAATTCTTATCATTTCTTCCATAGCTTTCAATATATTTATAACTTATTTGTAATCACTCTTATCAC
MARKER 50378 (SEQ ID NO: 83): A→G
TTGAGATATCAAATCAAGCGTTGCATATTTATAGTACACTGGTGTAGCTGAAATCGCGAAGAGAACACGAAAATCAG
AGAAGTCAATGGTTCCTTTGTGTTGGATTTCACATGAAAGCATCCTTATGTTGTACATGCGTGATTACAATATGATA
CAAGATGTAAGCTAAAAATTGTTTTATCTTTGTCTATGAGATGTAGTTCATACTCTATAATAAAGTCCCAACCCTTA
ATTCTCATATTCACAACCGTATCAGAATCCAACACCAAACCATTATAAAGAATGTTCTTCGTCGAGGCG
MARKER 51565 (SEQ ID NO: 84): C→T
CCACTATCGCTTACACTTTCTTTATCCTGTTCTTCTTCATCTTTCGTTTTGGACTTTATTTTACTGTCAGGTGACAA
GCAAAGTAACGATGTTGGACTTTGCGAAGATGTGGATGGTACGCTAGAAAAAAAATGAGGATTGGTTAATATGTCTA
ATTATTACATCGCTTTTTTTTAAATCTTTTCTAAAATTAAACTGAATAATCAACTTATTTGCTATTCAGTTTATCTT
ATTTTTTATCAACAAAATTCGAGGAAACAAATCGCTTATCAGAATAATTGTTTTGATCAACAAATAAAG
MARKER 58162 A (SEQ ID NO: 85): G→A
CAATCCCACAAATTCAGTGTGTCGGCGGGTCAGCGAAGGGAAAGTTTGAACCGAGGGTATGTACAAATTGTGATAAT
TTTGTGATGACGTAGTAAATTTCATAGTTTTGCATGCTTTAATGTTGATAGTCGCACAATCCTACGTTGATTAAATT
TAGCTATTAGATATCCTACTAAATTATGTTGTTCATAATTTTTGTTTTTAAAATGCTCCACTTATATTTTCAGGTTG
TGCAGTGCTACAATAGGGGTTATGACGGCAATGATGTCCAATGGGAGTGTAAAGCGGAAATGAGCAATC
MARKER 58864 (SEQ ID NO: 86): T→C
TCAGATAAATTGTATTTGATGTTAATTCAAAGAAGAAAAAAATAATCAGTAGAATATGAATCGAATAATATTCATAC
AACCAGTTTATTCATTATTATTCACTTTTAACGTCTAAATGACGTAGCTACGCTTTTTTTCTCGCTTTCAAGCCTTT
ACTGACCAAGATTAATGTACATTCTGTTGAACAAGATTAATCGACATTCTATCGATCAAGATCAAGCTTTTACTGAT
CAAGATTAATAATGACATTCTTCTGTTGATCAAGATTAATCGACATTCCATTGATCAAGATTAATCGAC
MARKER 62666 A (SEQ ID NO: 87): G→A
CTCTCTAAAACCTATTGGTCACTAAACTTGCACTGACTAAAAACTATTGGTCATCAGACTTGTGATTCATTGAAAAG
ACCGTTAGCCGCTAAAATTATGATTCACTAAAAAAAATCTATTGATCATTAAATCTGTAATCATTGAGAAACTACAA
TCATTGGTCATTAAGTTTGTGCTCTCTAAAACCTATTGGTCATTAAACTGACTAAAAACTATTGGTCACTGAACCTA
GAGTCTATTAAAAAAAAAATCATTGTATCAATAAATTTATTGTTTACTATCAAATCCATTGATTACTGA
MARKER 62666 B (SEQ ID NO: 88): A→T
TCTAAAACCTATTGGTCACTAAACTTGCACTGACTAAAAACTATTGGTCATCAGACTTGTGATTCATTGAAAAGACC
GTTAGCCGCTAAAATTATGATTCACTAAAAAAAATCTATTGATCATTAAATCTGTAATCATTGAGAAACTGCATTCA
TTGGTCATTAAGTTTGTGCTCTCTAAAACCTATTGGTCATTAAACTGACTAAAAACTATTGGTCACTGAACCTAGAG
TCTATTAAAAAAAAAATCATTGTATCAATAAATTTATTGTTTACTATCAAATCCATTGATTACTGAATA
MARKER 7060 (SEQ ID NO: 89): G→A
AAAATGTATCAAATTCTTCGATGCCATAAATTATACAGACTTGATTGGCATTTTTTCTAACTTTCATCATGAACCAT
TCTATTTCTAAATTGATCCATTACAAAATCAACTTTGTGATATCATCAATCTCAGTCATAACGAGAAATAATGATAA
TATAAAGCGACTATCATTTGAATTTCCTGAATATTCAAGATGTAATTACATCTTTTTTTTAATGTAATCAAAATTTC
TTGCCATCAATAATTTTTCAACATATGCTTTCATCGACTGCCTTATGCAGATCGTAATGATGACAGCCA
MARKER 12056 (SEQ ID NO: 90): T→C
ATTGATTAAAAAGAATCAACATTAAATTTTTGATATAGTCGAGAAATCCTTCGTGATAATTCTTTTAGAACAATTCT
TTACACTAAACTTGTATTTACTTGCTTATTATTTGTCTAAAGATACTAACTATTTGTCAGTGGAATTTATGATCTTG
GCATTATTGCATATAACGCTTTCCTAAAATCTGAAATTTTTCAGTATTTTAAAAACTAAGACGATTATTAAATATTA
CTCAAAGCTTAGAACTTTGATTATACTAATCAAATCAAAAATTTCATCAGCGATTTTTGTTGTGTCATT
MARKER 16261 (SEQ ID NO: 91): T→C
ATTTTTTCCAGCAGAATTGTCATCAAAAATCCCATTTTTGATATCCTCTTCATCGAAACTTGCTCCTGAATCCAGAG
AACAACGAAGAATGTGTAAATCTATTTCAGTAGCCTGCTCATTGTGCAATTCAGCGACTTTATTTCTGTGCTTCAAG
CTAACTTCTTCATTATGCCACTCCTCTTCTCTCGCTATTTTTTCGCTATCTAATTCAAAATCTTCGTCTGAAACGGA
ATCAACTCCTGACGATGTACTCGACACTGATAATATTTTCATGCCGATTTTTCTCTCAAACGAATCTTT
MARKER 23195 (SEQ ID NO: 92): C→T
GAATGAAGAGCAAAAAAATAGTCACGACCACCTGCAATAAAAACAGCATCTCCGTAAAAATGATTGAATTGATTCCC
GAAATACGAGTTTATCAAATTGAGAATTATGCAAATTAATTATCAGCATGCAGATTTACTGATTTTATATCTCTCAT
ACCGAAATTAAGGTGATGTTTTCCATTTCTTTGTTTCCACAATGTCTTCTTTGTGAATCGTTTTGGATCAACTATTA
ATCCGATCGAATCAATCCTCCAAATATGAGTTTATTCAACGTAACAAAACATTGTCCGAGATAATCAAA
MARKER 28579 (SEQ ID NO: 93): T→C
TGGAAATTTCGAAATCGAAAGGATGAAGAAAAAGGATCCTTGATCTATACATTAAATATCACCATATCAACTAGCAT
GGCAAGTCAAAGTAATGTTATCATTTAAATAAAAAAGATGAATAGTAGGACTACAGGTTATATTGTTAAAAGTCGAC
AAATTTGGAGTAATTGACAGAGATCAACGATTAAATGTAATGGATGATCTTATCTTCTTTTTTCAACTACGCCAAAA
TGAAAATAACAATTGAATTTGTCGAATAAGAAACTAACATTTTGAAAATAAGATTGAACATTTATAAAT
MARKER 48869 (SEQ ID NO: 94): G→A
GGTTGGATCATTATCGACAGAACTTTAGAAGTTTCTTGATAAGGACGAAAAGAAGCAGCACCATTGCTGATCTAAAC
AAGGAAAAAAGACCTTTTTTGGAATATTGAAGTTTTTACTGATAGGTGCGTGCTGTGTACTGTGGGCATAAGTACAA
GCTTCATGCTCCGCAGCGTGAATACGTGCTGCATGCATACTATGCAGTAAAGGTGCGTGTCGTATTGCTCAATAAGT
GTATAAATTGCTGCTTTTCTTGCATAGTTAAATATTTTGTTTTCATTTTTTCCGCTATTCAAAATAAAT
MARKER 53021 (SEQ ID NO: 95): G→A
GTTGGGATTTCAGACTCTCACTCGGTGTCGTTTCACAGTGATATCTGAATCGAAGTCACAAGCAGGTATGAATGCAT
AACAACTAATATCCATTGCAGAAACAAGGCAAAACTGAGAAGCTCGAGCAATATAGCTATAGAAGCTGGTACCACAG
ATGACATTACATGGTATTTCCATTTCAGCTTCACAAACATTGTAAATAGCTTGCTTCGATGATTCAATATCTCGTTC
TACGATATTCTTAAAGTAATTTTTATTTATTTGAAGTATAGATTACATCCATGTTCTATCTATCATTTC
MARKER 7986 (SEQ ID NO: 96): G→A
TGTTCTGAACATCTCTTTTTGATTATCTTTTTTAATTCCTCCATTATTTTCGTTTTTTTCGTTGTGAATTAATATTG
TTTGTCTTTGATTCAGATGATATTTTCGGATCGTAAATAGATGGCATCGGCATAAGCGTATTGAGAAGCATTCAATG
GTGCACTCTTGCTTCTTTTTTTTTTGAAATCTTTCTCGATAATCAAATAAGTGCAGGATGCCAATCATTAACAATTT
CGTTCCACTTTTTCAGTTCTTATTCTTATAACACCACATCTCATTTGCAATTTTGTCGCCAATGATTTT
MARKER 48094 (SEQ ID NO: 97): C→T
TTTTTTCGAGGTCACTCTGGAAAAATAAATCATATTTTAAAAAGACATAAAATAAAAAATATGTATATATAAGAAAA
TTTTTACTCTGAATTTCTTAAGAAAATTCTCGATTCTGTTTTCCATAAATTCCGGAATATGTTGTCCCTGAATTAAG
AATTCGATTCCTTGCACACCATTATTTCGTCTAGTTCCTGTGTGAACAATGTAACCTGGAAATGAACACATAAACTG
TAATATTTTGAGCTTAAAATAATTATGAGGATGCGAAACTGAAGATATTCATAAATGTTTAAAAAAAAA
MARKER 6568 (SEQ ID NO: 98): T→C
GTCCATGCATTGCTTTTCGGAAGTTAGTGTAGATTCAGTGAATATTTAATACCAGTCTCTTTCTAATTCAAAAGAGC
CTCCCATTTCTTTTTTCAGTTTCAGTCTCTGAATCAGAGCGTGTAATCTACCACTCCATTGCCGAAAACAGCTCGAT
GTATTTCCTGCTACGTAGTGTTTAGAATTGGCGTATGCCACTTGCTCATTATTCGCGCATGAAGTGTAACTGTGAAT
AGAATGATACTACTGTTAGAAGAGAATGCGTTCACTTTATTTAACATTATACTGATTCATTTCTTCTTT
MARKER 17022 (SEQ ID NO: 99): C→T
AGTGAACGAGAAAAAACAGAAGAAGAGATAGCACATCAAGATCGTGAGAAATTAATTAGACAAGAAAAAGCTCGTCT
TACACAAATATATCAGGTTTTCTTTTTCTTGCTTTCGAAAGTTATTTGAATTATCTCATTTCTTTGAATTTTATAAG
AAATAATTTAATTTTTTTTTGAAATTTTGCCTATTGAGCTCTAAATTTTGTAAAAAGTTTTCTAGGATGATGTTAGC
AAAGCAAAAAAGAAATCCAAAAGTGATGGTAACAAACAGGAAGATTTTATAGTGAGGTACGATAATACG
MARKER 55751 A (SEQ ID NO: 100): A→G
TAGACAATATCATCCTTCCTTTTTTTTTGCTCAATTTCTCTGCTCATTGCTTTGATGATAATGGTAGGTGGTATAAT
GAAACGAATAGATAATTGATGTTCGCAAACATTTGCTGTTAAATTTCAGTAAAGAAATTGACCTTTTTGCTTTGTGT
TGGATGTTTAGCTTCATTTTCTTCTTGTTCATTGTCATATTCATTCTCTCAAAACTTCTTGCTTAGCGATGCTAATA
TAAATACTGGAAGAATGCCTTTGCTTTGTTTTAGTTGTAAATCATCACCAAGGTATTTTTTTGCAAAAT
MARKER 55751 B (SEQ ID NO: 101): A→G
AAGATGAAACTAAAAAAAATTATTTCGAAAAAAAGAAAATAAAATTAATGAAATAAAAGCAAAAATGAACAAACCGT
ATTAATTTTAAACAATAAACAATATCGAAATCGAAAAATGGACTATTATTGATGAACTATATTTTCAAAATGTGAAA
GGTCAAAGTTTGTTTCAATTATGATAAATACAATTTAAAATAAGATTAAGCTAACAAATAAGTTGAGCAAATTGATG
AAACAAACAAATCAGAATATATTACAGAAAATGATATAACATGAAAATATATTAGACCAATTATTTTTA
MARKER 15893 (SEQ ID NO: 102): T→C
TTGAAGTTTTCAGATAAACTTTGATAAAAAATTGTTCTATGAATTCTCAAATTTCAATTAGTGATACTTATTTCGAA
GGTAATTATGCCTGATTGAATCTTCAATATCAACAAAATGAAAATTTTAGTATGATTGTTAACTCATACACCTCTAA
TTAAAGGTATTTTCTTTATCCCATGAAATGAAAATTTATTAAGAACTTAGAAAGCTACGGTATGCCTTTGATGCAAA
AGAAAGATTCATTTTCATTAAATCATGTTTAAAAAAAAGAGCAAAGAGCAAAAGGTGATGAAAGTTTTT
MARKER 25462 (SEQ ID NO: 103): C→T
TTCTATACGAAATATTTGTCTGCCATAAATCTACTCAGGAACTCGATACATCAAAACATAAGTACGCTTGCTCTTTA
TTTTTCGTTTGAAAAATAAATAGATCATTTTCGCACTTACATTTCAATTTCAATTGCTTTATTCATATCTTTCTGTT
TTTACTTACTGGTATTTAACAGTCGTTGTTCACAATTTAATGATCTATGAAACACCATTTAATTGTATTTGGACTAA
CTTTTCGACAAGCAAAAGATTAAAATTGTCTTCAGATACAGTTATAAATTTACATTGAAGATAAATGAA
MARKER 33494 (SEQ ID NO: 104): A→C
TAACGATCTGTATATCAATGGAATAATATTCAGTTCATGTTGTACTCGATATGAGATAGAATTACAATTTTGGAACA
AGATAATCTCAACAGCTATTTTCAAGAATAGTTAAATTAGGATACCATTCAAAGAAACTTTAAAAAATGATTTCCAT
ACATTAATGCTTTTTGTGTTTTCGCTCTCGACCAGAATCCAGGAATTGTCCATTATCATCAATTTGATTAACTTTTA
TCTTTATTCTAATTCTTCAACATTTCTCTAATTGATATTAGTTTCAATATTTTAATAAGTAAAAATTTA
MARKER 17935 (SEQ ID NO: 105): T→C
ATAATGTGTTATTGATCAAAGGATTTTTAGTTACCTACCAGATGGAAAAAAAGCAAGTTTACGAAAACAGAAGTTAG
CATCAACTTTCATCCATGGTTACACCGTATATAATCCAATCGACTCATACTTTATGTTGATCTGATTTTATAGCAGA
TAACTAGTTACCTTGCTCAGCAGCAGCTAAATCCTTTCTATTTGCTTAATAACAGAAATATTTTTCATTAACAAAGA
AATTATACTCCGTGTTTGACATTTCATTTTAATTTCGTTCCAAAAATGAAAAAAGCTTCGTCCGGAAAT
MARKER 48561 (SEQ ID NO: 106): C→T
ATTATTTTGTAGTTTTTCATTTTTTAGTTCAATTTTCCTTTGCTTATTTTAAATATGCCATTCTTTATTCAGACTCA
TAGCGAATGCATATGTTCATTAATTTTTTTAGTTACAGTTACAAATTCTCAATTTCTCTTTAATCATTTTTTTTTCC
AAAAATAGTCTGAGCACTCAACCATTCATTCAACAATTGCAGCTTTTTTTATTGGAGCCTTGTCAAATTATCAATTC
GTTTCCATGTTTATTATTGAAATAATAAACGGTATTTAGGATAACGAAGTTCGCTTAGCTTCTTTGACT
MARKER 42003 (SEQ ID NO: 107): T→G
AAAAATTCAGGTAATGAGATCAGTAATTTTTTTTGGTCACTTTGCTGTTTCTTATCAGCTCATTGTTATCCATATCA
AATGAGCGAAAGTGTGTATCACATATTGGCAGAGTGTAATCTATGAAGATTTTGCGTATCAAAGTAATTATGAGAGA
ACTGATAATTTTATTTTAAAGTAGTAGAAAACTCGAATTAAGCTAATAAATAATCGGTTGATATCCATGAAATGAAT
TACTAATGAAATGGATAATTGAGTAATAACAAATGATATTCATGAAGAAAGGCAGGTTTTTTTTAATAG
MARKER 29566 (SEQ ID NO: 108): C→T
TATACTTAAAACAAGAAATACAATTAATGCCAATAGCAGAGTGAAACTTCTGAAAAATAATGAGTTGAAACTGGTAA
AATTAACATTTTATTAGAAATTTCAGAAACTTATGACTCCTCATGGCACTATCACAAAATGTTTGAAAAAAATTGAC
AGCTCGCGTCGATTGCAAAAATCATGATTCCTGATATTTAGTATCGAACATGTGACAAATAATATAAAGACCTAACC
ATAAAGCACTGAAACAACTCGCGGAAACAAAAAATTAATTTGCATAAACACGGAATACGATCAGAAAAT
MARKER 33868 (SEQ ID NO: 109): G→A
GAATTTTTTTAGAAGGCTTGAAGTCGAGAATATTAGAGACTATATCGAAGACTTAAATAATCCTGGTAATCTTCTGT
ATGAATCAAAATTACCTCGAACAGAACCATTCAGCACATCACGAGATAATTCATGGAATGAAACTAGCCAATCAGAG
CGTTGTAAAAGAAGAAAGTTATGAAATGACCTTAAAATCAATTTAAAGCATGTCCTCGCCATATAAGCGTTGAAAAG
TTAGGATAGAATCAATTATCAAAAAAATATGTTAACTAGATCTTATCAATCAAAACATCAGAAGGAAAA

In another example, genetic markers from D. immitis include the sequences below (SEQ ID NOs: 110-127), where the underlined nucleotides (i.e., the polymorphic sites) indicate the SNP nucleotide position within the fragment that correlates with resistance to MLs (i.e., the alternative nucleotide). Those markers were identified after genotype frequencies comparison between susceptible individuals and confirmed ML resistant individuals. In these sequences, the underlined nucleotide at the SNP position is generally different than the nucleotide found at this position in organisms that are susceptible to MLs (wild-type). In the sequences below, the nucleotide at the SNP position in the indicated sequence correlates with resistance to MLs. In the heading for each sequence, the nucleotide change from wild-type to the alternative nucleotide (alternative nucleotide correlates with ML resistance) at the polymorphic site is shown.

MARKER 31307 (SEQ ID NO: 110): A→G
ATATGATAATAGTGAAACAATTCCATCACAATAAATATTATCGATTAGG
AGATAAATTAACATTGATGCCTCAATTTTGGTCAACAATATATATTTGC
TATTAGCATTTTTATTAAATCGTTTTTATCTGACTTGACATAAATTGAA
ATAGAAAAAATTGAATCTGTTCCTTGTTAGATTTTCTTCTAAAAATTCT
TGAAATACAAATAATTTCTTAAATTTCAATATTTCTACATAATGTATTG
CGACAAAAATGCTAATGATTGGCTTATTATTATTTCGAATAATTTTTTA
ATCAAA
MARKER 26225 (SEQ ID NO: 111): A→G
AGCTCGAAGATCGGACAAAATTTGTTCAGCTTGTTGCCTTGAGGCTTTA
GTCTGAAAAGACACTTAAAAGTATAAACAAATTATATTCAAAAAATCTT
ATTTTGCATTTGCGTCTTAATTTTTGCTTTTTGCAAAGTTTTTTCCGAG
CAAGTTTTTCTATCTTCGAAAAGATTATATCAATTAAAATTTCAATTTA
AGCAATCATTGCCTCTTCGAGTTTCTGTTTCAGCAAATAAATATCACCA
CCACGACGCTGTCGGAAGAAAGAAACGCCTTTCCCAATTTCTCGTCTCA
ACTTTT
MARKER 47722 B (SEQ ID NO: 112): A→G
TAAGAAAGCTGGGAGATTTTCCAAAAACACTATTTCCCACGATTTGTTG
TTTTCTATGATCAATTCTTAATCAAACTCTGAAATTCTCAAATTTTCGA
TTTCTATCCAACTTCTACATATTTTTTTAGAAAATTCATATTTAGCAAA
GCTGAGTGTAGAAATAATTCATACTTGCAATTCATTTTTCTTAAATTTT
CGAATTTCTTAAAAAAGTATTTCAAATTACCTACCAATTTTGATTGGAA
AATTCGTGGATGCTAAAAATTCAAATCAAAATAGTTAAACAGTATTCCT
AATTGT
MARKER 58162 B (SEQ ID NO: 113): T→C
AATTTAAAAAACACATCGACATTTTGCGGTACGGTAATGATTGTTTACA
GTAACTAAATGTGTCCTACGGTAGTAATACTCGTGTACGTAATGAATGA
GTATAGTGACCGGATATTTCCTTCACTAGTAGGCAATATTAAGAAGTAT
TTTCATTTTCATATTCTATCTAAAATAAACCGATAAAATGGTTTTTGAA
TTATTACTTTTTGATTGTTATTTTTTGATCCTAAATTGTAAAATACTGT
AATAATTTAGCTAATTTCTATGATTCTATTCAATATGCTTAAATTAAAA
TTCTAA
MARKER 17709 (SEQ ID NO: 114): T→C
TCGTATTTGTTGTATGTAATATAGAAATATTGTTTAAATTCAATATGTA
GAAAAAATTTCTANNNNNNNNNNAATTAATTACATATTAACTCGTATTT
GTTGTATGTAATATAGAAATATTGTTTAAATTCAATATGTAGAAAAAAT
TTCCATAATAAAGACGAACAGCATTTATAATTATCAATGATAAGTTGAA
ATTAATTCATCAATGATAAGTTGAAATTAATTTATTTGAAATAATTTCT
TTGAAATTCGAATATAGACGAGAATTTTTTTTTTTTTGCTAATCGTTTA
TCAAAT
MARKER 47141 (SEQ ID NO: 115): T→C
TCTAGCAATATAAATTACAAGAATATGCCGTCCAAGTATTTCAGAATTT
ATTATTAATTTGGATAATAATACATTGTAAATACTGCGTATTCTGGATT
ATTATGCACTGCATAATAACATGCAATTTCGTCTACATATCGCGAATAA
ACGCCAAAAGATTTCTCGATAAAAGAAAATATAAGAATTCGTAAATGAA
TGTTGTGTCAGAGATATGTGTTAATTCATAAGTCAAGATGTTGTAAATC
GATCCATATTAGTAATCATATTTACGTGCTCGTAAATAAAAGCGGTGAT
TCTTGT
MARKER 48750 A (SEQ ID NO: 116): A→G
ATCGAAAAAAGATGATCTGATGACGGAAGGCGAAATGTCTGCAGAAGCT
AAGATGACGGAAGAAAAAAGTGAAGAAATGAAAGAAGAAGCTGGTAAAA
CTCAGAAGGAATGTAAAACTGGAGAATCGAAAAAAGATGATCTGATGAC
GGAGGGCGAAATGTCTAAAGAAGCTAAGATGTCGGAAGAAAAAAGTGAA
GAAATGAAAGAAGAAGCTGATAAAACTCAGAAGGAATGTAAAACGGAAG
AATCGAAAAAAGACGATCTGACGACAGAAGGCGAAAAATCTGAAGTAGA
TGAGCC
MARKER 63962 (SEQ ID NO: 117): A→G
ACTAATGATAAGAAACGGAGCCGACGATTTTAGGAAATGAATAATAACG
ACATTGACAACCATTGTTAGAAAATTGATAGTACTGATAATAAAAGCTA
GTTATAGAAAATTGATAATAATAATAAAATTGCTGGTAGCAAATGTCTA
GAAGTGATAATAAAATTAATGATAGCAAATGGATTAGCAATGATAATTA
AACTGATGATAGCGAATGGATTAGTAATGATAATAAAATTGATGATAGC
AAATGACTAATAATGGTAATAAAAGTTAATGCTAGTGATAACTTGTATT
TTAAGT
MARKER 6372 (SEQ ID NO: 118): A→G
ACAGTTTATAGTTACAATATTCTCCGGTGACTAACTGTATTTTACAACT
TATAATTATAGATTACAAAATATATTATAGTAGTTTTATAATTACAGTA
TTCTTAAGTGAATAACTATACTTTACAGCTTACAGTTACAGTAGTTTTC
TATGTTTTTGAATATTAATTTTACATGGTTTTTCCTAGTTTCAGTTTCA
AAATTTTCAGATATTTTATGTGTTAAAGCAAATTATATTCGAGATATAA
AAAGTACTGGTCATATCTTACAATTCTCATCCTTCTATATTGGAAAGAA
TTGAGT
MARKER 15611 (SEQ ID NO: 119): T→C
GTATTGGGACCGCGTATCGGGAAATCTGAAAGAAGTCTTTAACAGTATT
TTAAATGAATAATTCAAATCGTTACTTCTTAATATATTAATTTATGCGT
ATATATGCAGTACATAGCATTGCTTAAATTCTTATTTTTCCGCGGTTAA
AACCCTATGTAAGATAAGGGAGGTGATTGTATCTGCGCCGTACTCCTTG
TTTTAATCTACCTGCTTGTTGTATATCCTCCACATATTGTAACTGCAGC
TTCACATTTGCATATATAGTAAGGGCATCGTTGTCTCCAGAAGAGATAT
ATTATC
MARKER 46432 (SEQ ID NO: 120): T→A
GCTGCCCGAATGTTACAATTAGGACGAAAGTAAAAGTAGTTGACTGTAG
GTATGACGATAAAGGAAAAATTTGTATCTTAAGACTTTACAATTTCTAA
ATATTACGTGTTTTATCGTGCTAACATCACGAATTCCATATTCACAAAA
AAAATTTTGTAGAACTCCATCTGGTTTGGATGAATTTGCTACAGTTGAA
CTGGATGATGGAACGAAATTGCAAACATCTCTTATTGTTAGTATTTTCT
AAATTCTGTGAAATTTTGCAACGGCATTCATGTTTAATTATTAATTTGG
AGAAAG
MARKER 29594 (SEQ ID NO: 121): T→A
AAATAAGCAAATCCGAAAGTATTACATATACGGACTAAATATTGCCATT
CATTCGGGAGTATACCATTGCAACCATTGGTATTTCATTTGATCGAGAA
AACTAGTTTTTGTAGTTTGGGATAAAGAGAAATGGAGAGAGGAACTTTC
ATGATCAATTTCTTTACGTACTGAAATTCATTTCTATGGATGTTCTTTT
TCTATTTCATTCTCCTCAGCAAATACAGTCCGAACAGTCATCAAATAAG
TCTAAAAGGCATGAATAATATAAACATCAGCAACTTTTTAAATGAATGC
TTATTA
MARKER 26784 (SEQ ID NO: 122): G→C
ATTTCTATAAACATCTCTTGCATTGATTAATTTAACATGTTGCAATAAA
TATTTCTTACTTTTGAATGTATCATTTACTAGAAAAAACTTCAATCGAG
GAAATAAGTTTTAAAATAAATTCATATTTGAATTCATGTCAGTTCAAAA
ATTCTATTACTATAATACATGTCTCTTGGTTGTATCTTTTTTTCTTTTG
AAATAATACAATCAAACGGTTTCCTAAATTTTCATAGACATCATATTTT
AAAAAAAAATGCATTTGAAAATTTTCGAAAATCAATGAACTTAATTGAT
GAAAAA
MARKER 51661 (SEQ ID NO: 123): C→G
GCATGTGTATGTAGTATTTCTTTGTAAACAACATATCTAATCTGTCTGT
CCCTTTAACATTATAGAATAGTCAGTTAGTCCGCTATTTATTTTAATAA
CAAAATATCTCACTTAACTTCCATTTCTTTCCTAAATAATTTTGTTTCG
CTAGATCTTTCCTATAATTTTCAAATTTTCAAAAATGAATTAATCTTTT
ATTTATATATGTGTATGTATGTGTATGTATGTATGTGTACGTTGCATAT
ATGTATATGTATGTGTGTATGTGTGTATATGTATATGTATATGTGTGTA
TGTGTG
MARKER 7819 (SEQ ID NO: 124): G→C
TATGCATAATGTGCGACCAGCCAATAATGTCTTCAAACCATAATTATGC
AGAAATAAATTTTTTCCAGAAATAATTTTTTTTTTTTTACATATACTTC
CGATCTGTGAGAAAATACATTTGAAGTGAAGTGTGAAGCAATGCTACTT
TTTCAAACAACATTGTGAAAATGGATTAAAACGCACCAATGGAGCAAGA
GATCGTAAGTTTCGTTCCGCATGTCCTGTGGCAACGTGTAAACCATCCG
TTAACGATATATGATGTAAAAGCCGACACACCCAAATTAAAATCCATTA
TAAACA
MARKER 26704 (SEQ ID NO: 125): G→C
AAATGGATCGTATTCACTTCGTAAGAACTTAGTGAACGAAAAATCAAAC
CATCACAATAACTTTACTTTTTTTCTTTTTTTACTAAACACACTATCCT
ATGAAAACAAAATGTCCAAATAGATTCATATGATAATGAACTGTGAAGT
TATCCAATCTATCAGTTCTCGAAGAGGGAATAAATAAAAACATTAAGCA
ACCCACCGATCTTCGCTGACCATCTCCTTCTTCATTAGCAAGAAGCAAA
TCTTGTGGTGATATTTCTGCAACCATCTGCAAAATAAAGCACGAAAAAT
TAAGGA
MARKER 14329 (SEQ ID NO: 126): C→A
TTTGATATGCAATCAACTAACCAAATCAGAATTCAATGCATTCTGATAA
ATTTCTTCAATATCGTGCATCAATTCGACATCATATTTTGACAGTGATG
CTACCTTTTTAGCCGTATTTCGGAAAAATATGAATTCAACCAGCTGCGT
CCCAAAATTTAAGGCTGTAGCAAGTCCAGCAACAACCAGCCCTACAACT
GAAAATTCTAAAAACTGGTTCACGTGCTTATCATTAATAATTTCAACAC
TATCACTATCTCCACATGAACTTGATCGATTATAATTTAGTAGAACTGA
AAAAAA
MARKER 56169 (SEQ ID NO: 127): T→G
ACAAATTCGTTTTAATATTGGATTACATTGAAATTGCTGAAATAAAGTG
GAAATATTGAAAAGCATTTTACAATATTTGTTAACAACATTATATTTAA
AGAATATACACCTTGGTTTAAATGGTAAAATAATCTCAAGAATTTTCAT
TAGGTTAATTTTTTTTTATTTATTTATATTCACAAAAAATTGTAAAAGA
AAACAAAAACAACAATAATAACGGTGACAACAACAACAATAATAATAAC
AAAACTATTTGTTGTGATTTTGCAGCATTGATGTAGTGGGGATCTTTTG
GAGCGA

The genotype frequencies for each SNP (SEQ ID NOs: 110-127) at the polymorphic sites are shown in FIG. 29 (Table 1). In one analysis, genotype differences of susceptible individuals were compared with confirmed resistant individuals. In a second analysis, genotype differences of susceptible individuals were compared with grouped confirmed resistant and LOE individuals.

### Kits and Methods

In embodiments of the invention, probes of the invention may be provided to a user as a kit. A kit of the invention may contain one or more probes of the invention. For example, a kit may comprise a probe capable of determining the genotype of a nematode at a SNP position in one of the fragments disclosed herein. The kit may further comprise one or more reagents, buffers, packaging materials, instructions for using the kit and containers for holding the components of the kit.

A probe of the invention may be one or more molecules that are capable of binding to, or associating with, the nucleic acid sample to determine the genotype of the nematode at one or more specific positions (e.g., polymorphic site) in the fragments disclosed herein. For example, probes may be used to determine whether a wild-type or alternative nucleotide is present at the SNP position of one or more of the fragments disclosed herein. An example probe may be a nucleic acid molecule or oligonucleotide. Example probes may contain a label or labels. Example labels may include radioactive labels, enzymatic labels and/or fluorescent labels.

An oligonucleotide used as a probe or primer may comprise any size, shape and composition that is suitable for use in the context of the invention. Preferably, an oligonucleotide of the invention may comprise DNA, RNA, synthetic nucleotides, non-natural nucleotides, altered nucleotides, or combinations of one or more thereof. In one embodiment, an oligonucleotide of the invention may comprise locked nucleic acids and/or peptide nucleic acids.

In embodiments of the invention, an oligonucleotide may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, or more nucleotides.

In embodiments of the invention, an oligonucleotide may encompass, without limitation, a primer or more than one primer, e.g. a primer pair, such as a forward primer and a reverse primer.

A primer may be an oligonucleotide that may be used to initiate DNA replication. Typically, a primer is a short oligonucleotide that may be about 10, about 15, about 20, about 25, about 30, about 35, about 40, about 45, about 50, about 55, about 60, about 65, about 70, about 75, about 80, about 85, about 90, about 95, about 100 or more nucleotides.

A primer may be used as part of an approach to detect the genotype of a nematode at a specific location of a gene. For example, a primer may be useful in amplifying DNA such as by PCR, RT-PCR and qRT PCR, for subsequent analysis, such as by Southern blot, sequencing, HRM (high resolution melt) or SSCP (single strand conformational polymorphism).

As used herein, an “aptamer” may be a nucleic acid or a peptide molecule that binds to a specific molecular target. For example, in solution, a chain of nucleotides may form intramolecular interactions that fold the aptamer into a complex three-dimensional shape. The shape of that aptamer allows it to bind tightly against the surface of its target molecule. Because of the diversity of molecular shapes that exists for nucleotide and amino acid sequences, aptamers may be obtained for a wide array of molecular targets, including, but not limited to, nucleic acid molecules, enzymes, membrane proteins, viral proteins, cytokines, growth factors, and immunoglobulins.

A probe of the invention may be prepared according to standard techniques known to a skilled person. For example, a probe may be produced synthetically, recombinantly or may be isolated from a natural source. In one embodiment, the source may be a biological source, for example, from a microorganism (e.g. a bacteria or a virus), an animal (e.g. a mouse, a rat, a rabbit, a goat, or a human), or a plant.

In the context of the invention, “a probe” may mean one probe or more than one probe. One or more types of probes may be simultaneously used in methods of the invention. Probe design and production are known in the art. Generally, a probe may be produced recombinantly, synthetically, or isolated from a natural source, e.g. from a cell, an animal or a plant. However, a skilled person would appreciate that probe production may depend on the type of probe at issue. A preferred probe may be a nucleic acid molecule (e.g. a primer), with or without a fluoroflor or dye. A probe may be linear or in the form of a hairpin, with a fluoroflor, with or without a quencher or another fluoroflor (e.g. for FRET analysis). It could also be an antibody that specifically recognizes the DNA (or protein) sequence. Another probe could be based on a RNA molecule. What would be preferred may depend on technical considerations, stability, cost, ease of use, etc.

In embodiments of the invention, probes of the invention may be provided to a user as a kit. A kit of the invention may contain one or more probes of the invention.

### Uses of the Methods and the Kits

Methods of the invention and kits to carry out the methods may have research, medical and industrial applications. The invention finds broad application in the management of heartworms in infected animals and in detecting ML resistant D. immitis nematodes in an area. Representative, non-limiting applications of the invention may include the detection, quantification and/or diagnosis of the existence of individuals or populations of D. immitis that are not susceptible to normal doses of ML for prophylaxis or therapy. In one embodiment, the ability to detect and quantify nucleic acid molecules of the invention is valuable insofar as it will instruct a practicing veterinarian to alter chemotherapeutic regimens for animals infected with D. immitis nematodes that have decreased responsiveness to MLs. Identification of ML resistant D. immitis nematodes may instruct a veterinarian to switch from ML therapy alone to therapy that may include an alternative agent or alternative agents, such as an adulticide (e.g. arsenic based drugs), diethylcarbamazine, antibiotics such as tetracycline, and combinations of one or more thereof in order to achieve cure and/or to minimize the spread of the resistant strain. Alternatively, a veterinarian may adjust the dosage of a ML and/or treatment regimen using a ML in the treatment of an animal infected with a ML resistant nematode. Typical recommended dose rates for ML preventatives include, for example, 6 μg/kg for ivermectin; 500 mg/kg for milbemycin oxime; 3 μg/kg (monthly) moxidectin; and 6 mg/kg for selamectin. A veterinarian may also combine one or more of the treatment approaches and therapies noted above in any combination suitable to treat an animal infected with a Diro.filaria spp. nematode, e.g. a ML resistant D. immitis nematode. For example, a veterinarian may treat such an animal with an adulticide, such as an arsenic based drug, and then follow up with a microfilaricide, such as a ML or diethylcarbamazine.

In one instance, an arsenic based drug may be used to treat an animal infected with a ML resistant D. immitis nematode. An arsenic based drug may include, but is not limited to, melarsomine dihydrochloride. Melarsomine dihydrochloride may be used, for example, at a dose of 2.5 mg/kg, twice, 24 hours apart. This may be repeated in 4 months depending on the response to the first treatment and the condition, age, and use of the animal. However, a skilled person would understand that the dosage may vary depending on the severity of the infection. For example, an infected animal such as a dog with severe (class 3) disease may receive one dose and allowed to recover for a few months before receiving the complete set of 2 doses.

In another instance, diethylcarbamazine may be used to treat an animal infected with a ML resistant D. immitis nematode. Diethylcarbamazine may be used, for example, at a dose of 25 to 50 mg per pound of an animal. The duration of administration may depend on the condition being treated, response to the medication and the development of any adverse effects.

In another instance, an antibiotic may be used to treat an animal infected with a ML resistant D. immitis nematode. Said antibiotic may include, but is not limited to, tetracycline. A tetracycline, such as doxycycline, which targets the Wolbachia endosymbionts in D. immitis may be used, for example, at a dose of 10 mg/kg/day for 40 days.

In a further instance, another anthelminthic agent may be used. Such other anthelminthic agent may include, but is not limited to, acaciasides. An acaciaside may be used, for example, at a dose of 10 mg/kg/day for 7 days.

In another embodiment, the detection of D. immitis nematode populations with the above mentioned genotypes may instruct the use of alternative agents, such as diethylcarbamazine as a prophylactic to protect susceptible animals, e.g. dogs.

In one instance, diethylcarbamazine may be used to prevent an animal from becoming infected with a ML resistant D. immitis nematode. In this regard, diethylcarbamazine may be used, for example, at a dose of 3 mg per pound of an animal once daily.

In another embodiment, a kit of the invention may be useful in as a commercial product in the detection of ML resistant D. immitis nematodes. Such a product may be suitable for use by, without limitation, a veterinarian, a physician, a pet owner, a farmer, a zoo keeper, an epidemiologist, or another consumer in need thereof.

### EXAMPLES

The examples are tor the purpose of illustrating an example and are not to be construed as illustrating limitations.

### Example 1—Susceptible and LOE Populations of D. immitis Parasites Used in the Studies

The various susceptible and LOE populations of D. immitis used in these studies are described below.
a. Susceptible isolates from Missouri. USA. Thirty five (35) D. immitis adult specimens were obtained from two dogs originating from an animal pound in Missouri. The history of the dogs prior to the animal pound is not known. The dogs were not subsequently treated. The D. immitis isolates were believed to be susceptible to ML heartworm preventatives.
b. Susceptible isolates from Grand Canary, Spain. Seventy-one (71) D. immitis adult specimens were obtained from 12 dogs originating from a shelter on Grand Canary. The dogs were never exposed to ML heartworm preventatives and heartworm prevention is not practiced in this region of Grand Canary.
c. Susceptible isolates from Grenada. WI. Ten (10) D. immitis adult specimens were obtained from 2 dogs originating from Grenada. The dogs were recruited from poor, remote areas of the island where ML heartworm prevention is not practiced.
d. Susceptible isolates from Italy, Six (6) D. immitis adult specimens were obtained from the Po Basin in northern Italy. D. immitis seroprevalence in dogs from this area is reported to be approximately 60-70%. ML heartworm preventatives are commonly given to dogs in this area. But, there are no reports of LOE (loss of efficacy) in Italy.
e. Loss of efficacy (LOE) isolate case 1. Microfilariae (mt) were isolated from a dog that was previously described (see Bourguinat et al.; W0201 1/120165). The dog was a male neutered Labrador mix, born in February, 2006, that weighed approximately 31 kg. He was a rescue dog from New Orleans, Louisiana, U.S.A., collected by the Boudreaux Rescue Crew, New Orleans, and subsequently transferred to Canada where he was adopted in January, 2008.

The dog was brought to the Main West Animal Hospital (MWAH) in Welland, Ontario on Jun. 6, 2008 (day 1) for a check-up. Blood collected from the dog tested positive with a heartworm antigen test (PETCHEK® PF; IDEXX Laboratories, Westbrook, Maine) and contained microfilariae of D. immitis. On Jun. 11, 2008 (day 6), initial work-up (bloodwork, thoracic radiographs, physical exam, urinalysis) was performed. Auscultation revealed a mild increase in bronchovesicular sounds in the lungs and a grade III-IV/VI heart murmur. The remainder of the physical exam was unremarkable. Thoracic radiography revealed moderate right-sided heart enlargement and an interstitial lung pattern in the caudodorsal lung field. These examinations indicated a diagnosis of class 2 heartworm disease.

Adulticide treatment was initiated on Jun. 11, 2008 (day 6) with 2.5 mg/kg intramuscular melarsomine dihydrochloride (IMMITICIDE®; Merial Inc.). The treatment was followed by two intramuscular treatments with 2.5 mg/kg melarsomine dihydrochloride on July 9 and July 10 (days 34, 35). Over the following 90 days, in order to eliminate circulating mf, the dog was treated on one occasion with milbemycin oxime (MO) and on two occasions with IVM (see Table 2). On days 159 and 160, four months after the last dose of adulticide, the dog was again treated with 2.5 mg/kg melarsomine dihydrochloride intramuscularly. The subsequent diagnostic testing and microfilaricidal treatments are summarized in Table 2. During the treatment of the dog, several heartworm antigen tests were conducted, including DIROCHEK® (Synbiotics Corporation, San Diego, California) and PETCHEK® (IDEXX Laboratories, Westbrook, Maine), which are microwell ELISA tests, and SNAP® PF (IDEXX Laboratories, Westbrook, Maine, a membrane format test designed for rapid in-clinic use (see Table 2).

To perform the Knott's test, 9 ml of 2% formalin and 1 ml blood (collected in EDTA) were mixed in a centrifuge tube. Centrifugation was performed in a LW Scientific EZ Swing SK centrifuge at 3000 rpm (604 m/s2) for 5 min. The supernatant fluid was discarded. A drop of 0.1% methylene blue solution was added to the pellet at the bottom of the centrifuge tube, mixed, and a drop of stained mixture examined under the microscope for D. immitis microfilariae. Table 2 indicates when this test was carried out and, when determined, the level of microfilaremia.

The dog was treated as follows. Two days after the last of three doses of melarsomine dihydrochloride in July 2008 (i.e., on day 37), the dog showed transitory signs consistent with death of adult heartworms (elevated rectal temperature, lethargy, cough, increased lung sounds). Beginning on day 41, these signs were managed with prednisone (Apo-Prednisone; Apotex, Toronto, ON, Canada), 1.3 mg/kg bid for 6 days. Following the administration of milbemycin oxime (MO) per os at 0.74 mg/kg on day 74, IVM per os at 50 ug/kg on day 95, and IVM per os at 200 ug/kg (4× the normal microfilaricidal dose rate) on day 125, the dog remained continually microfilaremic. On day 207, six weeks after the second treatment regimen of melarsomine dihydrochloride, on days 159 and 160, a Knott's test was still positive, so the dog was again treated with 200 μ/kg IVM per os. One month later, on day 242, a D. immitis antigen test was negative, which confirmed that the dog was free of adult worms. However, the dog was still microfilaremic. Thus, beginning on day 243, the dog was given MO per os at 0.74 mg/kg every 2 weeks on four occasions (see Table 2). Despite this, the dog remained microfilaremic on day 298. It was therefore administered MO per os at 1.1 mg/kg on days 298, 312, 326, 340 and 354. On day 356, blood was collected from the dog and examined: microfilariae were still present, and a D. immitis antigen test was still negative. On day 375, a blood sample was sent to Animal Health Laboratory, University of Guelph (AHLUG): microfilaremia was 6530 mf/ml, and an antigen test was still negative (see Table 2). As a result, beginning on day 384, the dog was administered MO per os at 2.0 mg/kg once daily for 7 days. On day 420, the dog had a microfilaraemia of 355 mf/ml. On day 420, the dog was again treated with MO per os at 2.0 mg/kg, and this was continued once daily for 8 days. Despite this second high-dose regimen, on day 480, while still testing negative with a heartworm antigen test, the dog had a microfilaremia of 1810 mf/ml.

Blood was collected from the dog on day 706 and DNA was isolated from pooled microfilariae.

TABLE 2
Diagnostic testing and treatment history for dog between 2008 and 2009

| | Antigen test | Adulticide | Microfilariae | Microfilaricide | |
|---|---|---|---|---|---|
| | Name-result | (melarsomine)* | concentration in | drug dosage | |
| Date (day) | (+ve or −ve) | dosage | blood (mf/ml) | (PO) | Comments |
| 2008 | | | | | |
| June 6 (1) | PetChek+ve^{a} | | Knott's test +ve^{a} | | |
| June 11 (6) | | 2.5 mg/kg | | | Classified as |
| | | | | | Class 2 |
| | | | | | heartworm |
| | | | | | disease |
| July 9 (34) | | 2.5 mg/kg | | | |
| July 10 (35) | | 2.5 mg/kg | | | |
| August 18 | | | | MO, 0.74 mg/kg | |
| (74) | | | | | |
| September 3 | | | Knott's test +ve^{a} | | |
| (90) | | | | | |
| September 8 | | | | IVM, 50 μg/kg | |
| (95) | | | | | |
| October 6 | | | Knott's test +ve^{a} | | |
| (123) | | | | | |
| October 8 | | | | IVM, 200 μg/kg | |
| (125) | | | | | |
| November 10 | | | Knott's test +ve^{a} | | |
| (158) | | | | | |
| November 11 | | 2.5 mg/kg | | | |
| (159) | | | | | |
| November 12 | | 2.5 mg/kg | | | |
| (160) | | | | | |
| December 12 | | | | MO, 0.74 mg/kg | |
| (190) | | | | | |
| December 29 | | | Knott's test +ve^{a} | | |
| (207) | | | | | |
| December 30 | | | | IVM, 200 μg/kg | |
| (208) | | | | | |
| 2009 | | | | | |
| February 2 | SNAP −ve^{a} | | Knott's test +ve^{a} | | Interpretation: |
| (242) | | | ≥100^{b} | | no adult |
| | | | | | heartworms |
| February 3 | | | | MO, 0.74 mg/kg | |
| (243) | | | | | |
| February 17 | | | | MO, 0.74 mg/kg | |
| (257) | | | | | |
| March 3 | | | Knott's test +ve^{a} | MO, 0.74 mg/kg | |
| (271) | | | ≥100^{b} | | |
| March 17 | | | | MO, 0.74 mg/kg | |
| (285) | | | | | |
| March 30 | | | Knott's test +ve^{a} | MO, 1.1 mg/kg | |
| (298) | | | ≥100^{b} | | |
| April 13 | | | | MO, 1.1 mg/kg | |
| (312) | | | | | |
| April 27 | | | | MO, 1.1 mg/kg | |
| (326) | | | | | |
| April 28 | | | Knott's test +ve^{a} | | |
| (327) | | | | | |
| May 11 (340) | | | | MO, 1.1 mg/kg | |
| May 25 (354) | | | | MO, 1.1 mg/kg | |
| May 27 (356) | SNAP −ve^{a} | | Knott's test +ve^{a} | | no adult |
| | | | | | heartworm |
| June 8 (368) | | | | MO, 1.1 mg/kg | |
| June 15 (375) | DiroChek −ve^{c} | | Knott's test +ve^{c} | | no adult |
| | | | 6530 | | heartworm |
| June 24 (384) | | | | MO, 2.0 mg/kg | |
| | | | | daily for 7 days | |
| July 30 (420) | | | Knott's test +ve^{c} | MO, 2.0 mg/kg | |
| | | | 355 | daily for 8 days | |
| September 28 | PetChek −ve^{a} | | Knott's test +ve^{c} | | |
| (480) | | | 1810 | | |
| 2010 | | | | | |
| May 12 (706) | | | | | Microfilariae |
| | | | | | collected for |
| | | | | | DNA isolation |

MO = milbemycin oxime (INTERCEPTOR ®);
IVM = ivermectin (IVOMEC ® Injection for cattle, sheep and swine, Merial Inc.);
*Adulticide = IMIMITICIDE ®;
^{a}= Main West Animal Hospital (i.e. test carried out in house);
^{b}= ldexx Laboratories;
^{c}= Animal Health Laboratory, University of Guelph.

f. LOE isolate case 2. Approximately 9000 pooled mfwere obtained from a dog from Mechanicsville, Virginia, that had been treated with INTERCEPTOR® from 2004 to 2008. In May 2008, the dog was heartworm antigen positive and was placed on HEARTGARD® Plus (IVM/PYR) for slow kill treatment. In 2008, the dog was still positive for heartworm antigen and was still microfilaremic. From Dr Blagburn's (Auburn University) in vitro assay: LD9s concentration for susceptible mf produced only a 10.5% kill, and 2× LD9s produced a 13.6% kill of mf.
g. LOE isolate case 3. Pooled mfwere obtained from low responder mf from an in vitro ivermectin susceptibility assay. The dog was a naturally infected client-owned animal, from Monroe, Louisiana, selected because it had been on ML heartworm preventative treatment. The veterinarian was convinced that compliance was not an issue. Patient records indicated that proper amounts of product had been provided to the client, based on numbers and weights of target animals in the household. The dog was microfilaremic despite the fact that it had been under ML heartworm prophylaxis.
h. LOE isolate case 4. Pooled mfwere obtained from a dog that had the history as described below. This stray dog originated from Haywood County, Tennessee, USA, and presented as heartworm antigen positive to a local clinic on Jan. 21, 2011. The dog was neutered on Jan. 26, 2011. On Feb. 1, 2011, doxycycline (200 mg orally twice per day) and prednisone (1 5 mg tablet orally every other day) therapy was initiated and continued for 30 days. On February 2, March 3 and Mar. 4, 2011, an injection of melarsomine dihydrochloride (IMMITICIDE®) (2.5 mg/kg) were given. On February 2, March 3 and Apr. 1, 2011, an oral dose of milbemycin oxime (INTERCEPTOR®) (11.5 mg/tablet) was given. On Apr. 5, 2011, a Knott's test was performed and was positive; ivermectin was administered subcutaneously at a dose of 0.26 mg/kg. On Apr. 11, 2011, Knott's test was again positive; ivermectin was administered subcutaneously at a dose of 0.39 mg/kg. Knott's tests were again performed on both April 19 and 26, 2011 and were both positive. On May 2, 2011, Knott's test was again positive and a blood smear showed microfilariae; ADVANTAGE MULTI® (2.5% imidacloprid, 10% moxidectin) was administered to the dog. On May 5, 2011, a blood smear was positive for microfilariae; at this time, microfilariae were collected. The repeated adulticide treatment led to the assumption they the dog was free of adult parasites. On Jun. 11, 2011, 200 mg of diethylcarbamazine was administered to the dog. No side effects of the treatment were noted. Within 7 days, the blood smear showed no mf. The dog was adopted on Aug. 18, 2011 and moved to Massachusetts.
i. LOE isolate case 5. Pooled mfwere obtained from a dog originating from West Monroe, Louisiana, USA. This was a veterinarian's dog. The medical history implied compliant use of milbemycin oxime and there were several negative heartworm antigen tests at annual check-ups, until a positive heartworm antigen test and presence of mf in the blood on Sep. 25, 2008. An in vitro microfilaria sensitivity assay was performed (B. Blagburn laboratory, Auburn University, Alabama) on Nov. 19, 2008. The results of the assay indicated drug-resistant organisms. Mosquitoes were fed on infected blood samples from this original dog. L3 larvae were used to infect a second dog. At the time of infection, the second dog had been under treatment with ivermectin. Thereafter, at weekly intervals, the second dog received 1 dose of 3 μg ivermectin/kg, followed by 11 doses of 6 μg ivermectin/kg, followed by 4 doses of 12 μg ivermectin/kg, followed by 8 doses of 24 μg ivermectin/kg (interrupted for one week after the 4th dose). During the entire period of weekly dosing with ivermectin, the dog was remained positive for mf. Microfilariae were collected at 1 and 2 weeks after the last treatment were used in the analysis.
j. LOE isolate case 6. The samples correspond to the second passage of parasite that came from a dog originally from Earle, Arkansas, USA. The original isolate LOE-6 dog received milbemycin oxime in 2004 and 2005, ivermectin/pyrantel in 2006 and 2007, and ivermectin/praziquantel/pyrantel (IVERHART MAX™) in January 2008 and at the beginning of July 2008. The owner stated that she had been consistent with prophylaxis. This dog tested negative for heartworm antigen at annual check-ups in 2005, 2006 and 2007. This dog was positive for heartworm antigen and microfilaremic at the annual exam on Nov. 4, 2008. Results of the in vitro microfilaria assay (B. Blagburn laboratory, Auburn University, AL) on this dog suggested resistance. Dog-LOE-6, was experimentally infected on Nov. 16, 2009 with L3 larvae derived from mosquitoes fed with blood from the first passage. The first passage dog was experimentally infected on Feb. 24, 2009 with L3 larvae derived from mosquitos fed with blood from a naturally infected dog (the original isolate LOE-6 dog).

### Example 2—DNA Isolation from Parasites Used in the Studies

Genomic DNA for the individual adult worms was extracted with DNEASY™ kit from Qiagen (Qiagen Inc, Mississauga, Canada). The genomic DNA extraction of individual mfwas extracted using QTAAMP® DNA Micro kit from Qiagen. To obtain enough DNA for analysis, the mfDNA was amplified using a REPLI-G® kit from Qiagen which allow amplifying the full genome from a very small amount of DNA. Mfwere isolated by filtration through polycarbonate membrane filters from freshly drawn blood.

### Example 3—DNA Sequencing, Analysis and Identification of SNPs

The goal was to identify genetic changes (e.g., nucleotide variations) present in LOE heartworm populations that were not present in the susceptible heartworm populations. Nucleotide variations in any of the LOE populations, as compared to a reference genome obtained from the susceptible isolates, would indicate potential SNP markers.

Initially, the genomes from the heartworm populations identified in lettered paragraphs a-h of Example 2 above (susceptible isolates from Missouri, Grand Canary Island, Grenada and Italy; LOE isolates cases 1-4) were sequenced using the HISEQ™2000 system from ILLUMINA®. Table 3 shows the number of reads and the number of bases that were sequenced for each population. Not included in Table 3 is information from heartworm populations identified in paragraphs i and j (resistant isolates from LOE cases 5 and 6).

TABLE 3
Read information on isolates used for whole genome sequencing

| Isolates | Number of reads | Number of bases |
|---|---|---|
| 1 - susceptible | 85,097,000 | 17,019,400,000 |
| 2 - susceptible | 78,242,862 | 15,648,572,400 |
| 3 - susceptible | 80,687,895 | 16,137,579,000 |
| 4 - susceptible | 75,515,617 | 15,103,123,400 |
| 5 - LOE-1 | 82,417,743 | 16,483,548,600 |
| 6 - LOE-2 | 74,261,369 | 14,852,273,800 |
| 7 - LOE-3 | 79,894,844 | 15,978,968,800 |
| 8 - LOE-4 | 75,477,318 | 15,095,463,600 |

The data generated from the ML susceptible samples (susceptible isolates from Missouri, Grand Canary Island, Grenada and Italy) were used to assemble the genome which was then used as the reference genome for the project. All of the individual fragments from the 4 susceptible populations were pooled together. Velvet aligner software (European Bioinformatics Institute) was used to assemble the genome. Reads were filtered by having the adaptor sequences removed/clipped, if found. Reads were trimmed at Q30 length 32 base pairs. A length of 32 base pairs is the Aligner seed default value and the number of reads was consistent with the default value. Table 4 describes the assembly of the reference genome used for the study.

TABLE 4
Information about the D. immitis genome assembly

| Number of contigs | 22 966 | |
|---|---|---|
| 50% of the contigs are longer than | 28 928 | bp |
| Length of longest contig | 250 211 | bp |
| Total bases in contigs | 94 611 006 | (94 Mb) |
| Number of contigs >1 kb | 6654 | |
| Total bases in contigs >I kb | 90 045 376 | bp (90 Mb) |

Once the reference heartworm genome was obtained from sequences of the susceptible isolates/populations, then the genomes from the LOE populations were compared to the reference genome, to identify differences and possible SNPs. As part of this analysis, genetic loci containing the potential SNPs were shown not to be significantly different between the individual susceptible populations (i.e., between the susceptible isolates from Missouri, Grand Canary Island, Grenada and Italy), as well as not to be significantly different between the individual LOE populations (LOE 1-4), but were significantly different between the susceptible populations and the LOE populations. To perform this analysis, the software program called PoPoolation2 (Kofler et al. Bioinformatics 27: 3435-3436, 2011) was used. The program required the use of other programs, such as Perl, R, bwa, and Samtools. First, a synchronized file was generated, which contained the nucleotide frequencies for every population at every base in the reference genome, after filtering for base quality, in a concise format. The synchronized file generated with the PoPoolation2 program contained detailed nucleotide count information on loci for each of the populations. P-values were generated with Fisher's exact test for all the possible comparisons between populations. To identify loci associated with ML resistance, p-values needed to be simultaneously not statistically significant(>0.05) within all susceptible samples and within all the LOE samples, and statistically significant(<0.05) between all susceptible versus all LOE samples. Three hundred thirty eight loci met these criteria, including 12 that had a p-value of 10-5, Flanking regions of 1000 bp including each locus that was statistically different between the susceptible and LOE samples were analyzed by Blast (BlastN and BlastX) in NCBI and in the Broad Institute filarial genome database to remove loci located in mitochondrial, Wolhachia or C. lupus familiaris DNA. Loci located in reads with very high polymorphism(>2 nucleotides and/or indels) or low coverage(<10×) were removed from further analysis. Nucleotide counts for each locus of interest were analyzed individually for the pooled populations to ensure that the increase or decrease in nucleotide frequency was in the same direction for all the susceptible samples or for all the LOE samples. The loci that best met the criteria were retained for further genotype analysis on individual parasites to assess actual allele frequencies in populations that had been characterized in terms of ML response.

From these analyses, 186 loci were found to be significantly different between the susceptible and LOE samples. As this approach was based on reads and nucleotide frequencies of pooled samples, these loci were further studied (SNP genotyping) using individual (not pooled) populations. For this purpose, SEQUENOM® SNP frequency analysis was used. Table 5, below, shows the origins of the DNA used in this analysis.

TABLE 5
Description of isolates used for SEQUENOM ® analysis

| | State and/or | # Individual | # Individual | From |
|---|---|---|---|---|
| | country of origin | adult worm | microfilaria | # dogs |
| Susceptible | | | | |
| samples = 181 | | | | |
| isolates | | | | |
| Sus1-Missouri | Missouri isolate, | | 49 | 1 |
| | USA | | | |
| Sus2-Missouri | Missouri isolate, | | 45 | 1 |
| | USA | | | |
| Grand Canary | Grand Canary, | 71 | | 11 |
| | Spain | | | |
| Grenada | Grenada, WI | 10 | | 2 |
| Italy | Northern Italy | 6 | | |
| Low responder | | | | |
| samples = 244 | | | | |
| Isolates | | | | |
| LOE-1 | New Orleans, | | 56 | 1 |
| | LA, USA, moved | | | |
| | to Ontario, | | | |
| | Canada | | | |
| LOE-2 | Mechanicsville, | | 35 | 1 |
| | VA, USA | | | |
| LOE-3 | Monroe, LA, | | 51 | 1 |
| | USA | | | |
| LOE-5 | West Monroe, | | 54 | 1 |
| | LA, USA | | | |
| LOE-6 | Earle, AR, USA | | 48 | 1 |

SEQUENOM® analysis is based on multiplex PCR and MALDI-TOF mass spectrometry. The SEQUENOM® analysis was used to evaluate the 186 loci using 425 individual samples (5 panels with 36-38 SNPs in each panel). Primer design for each SNP marker was based on a requirement that elongation primers be located in a non-polymorphic region 15 base pairs before or after the SNP of interest. All the genome calls were performed blinded (i.e., the sample origin and dog treatment history was not known during the analysis). A total of 79050 genotypes were analyzed. From the 186 potential loci, 109 were observed to have technical advantages to predict for ML loss of efficacy. The susceptible population carried more than 90% of the wild-type genotype while the LOE population had a significant lower genotype frequency of the wild-type genotype. These 109 loci are disclosed herein as SEQ ID NOs: 1-109.

### Example 4—Additional SNPs from Confirmed Resistant Organisms

LOE samples, as described in Example 1, were presumed to be resistant to MLs because of the history of treatment of the dogs with MLs and the continued presence of heartworm organisms. However, despite the history of treatment, an alternative explanation to true ML-resistance of the parasites is owner non-compliance of ML treatment. Therefore, a study was performed under controlled ML treatment conditions, to eliminate the possibility of owner non-compliance in ML treatment, as a possible reason for presence of heartworm organisms in dogs.

Heartworm organisms used in the efficacy studies were derived from one identified as Jd2009 from Earle, Arkansas, USA. Jd2009 received monthly MO in 2004 and 2005, IVM/pyrantel in 2006 and 2007, and IVM/praziquantel/pyrantel in January 2008 until early July 2008. Jd2009 tested negative for HW antigen in 2005, 2006, and 2007. This dog was heartworm antigen positive and microfilaremic on Apr. 11, 2008 despite a history of compliance with HW preventatives. Mf were obtained from the dog at this time with the consent of the owner and were sent to Auburn University, where the mfwere examined for sensitivity to IVM in an in vitro concentration-response assay measuring migration (Blagburn, B., American Heartworm Society-13th Triennial State of the Heartworm Symposium, 2010). These mfwere significantly less sensitive to IVM than mf obtained from a dog infected with a laboratory strain of D. immitis that was fully susceptible to the drug. The mf were used at Auburn University to infect mosquitoes to produce L3 that were used to infect dog Jd2009-1, which developed a patent infection. Mf from this dog were shown to be as resistant to ML as mf from Jd2009 in the in vitro migration assay.

L3s derived from mf harvested from Jd2009-1 were used at Auburn University to infect a second dog, Jd2009-2 and the dog was treated monthly with HEARTGARD PLUS® (0.006-0.013 mg/kg IVM) 9 consecutive times. Adult worms were recovered indicating that the Jd2009-2 isolate was resistant to IVM prophylaxis. In a second study, dogs were challenged with Jd2009-2 L3 on day O and treated monthly for 5 consecutive months with HEARTGARD PLUS® (0.007-0.009 mg/kg IVM; Study 1b). At necropsy on day 188, efficacy was 71.3%, confirming resistance to IVM prophylaxis in the Jd2009-2 isolate.

In another study, dogs were challenged with L3 on day 180 after PROHEART6® injection. At necropsy on day 150 after infection, efficacy was 21.6%, indicating that the Jd2009-2 was also resistant to the PROHEART6@long acting formulation of MOX, which has a claim for 100% protection for 180 days after treatment.

In another study, the confirmed IVM-resistant isolate Jd2009-2 was used to determine whether the resistance extended to other ML heartworm preventatives. None of the other ML heartworm preventatives (MOX, MO and SEL), given as monthly chemoprophylaxis as recommended, was fully effective, i.e., at least one dog in groups of four to six dogs on these heartworm preventatives became infected with D. immitis following treatment with each of these MLs used as recommended. DNA from individual organisms from two Jd2009 isolates were used. DNA from individuals from one group, called RES-1, came from 4 dogs from the PROHEART6® study, described above. DNA from individuals from another group, called RES-2, came from 6 dogs from the HEARTGARD PLUS® study, described above.

DNA was isolated from 115 adult worms and 79 mf from the RES-1 and RES-2 populations, as described in Example 2, and were analyzed using SEQUENOM® SNP frequency analysis, as described in Example 3. From this analysis, 18 additional loci (out of the initial 186 loci) were significantly different between the susceptible and RES samples. These loci are disclosed herein as SEQ ID NOs: 110-127.

While example compositions, methods, and so on have been illustrated by description, and while the descriptions are in considerable detail, it is not the intention of the applicants to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the disclosure is not limited to the specific details, the representative apparatus, and illustrative examples shown and described. Thus, this application is intended to embrace alterations, modifications, and variations that fall within the scope of the application. Furthermore, the preceding description is not meant to limit the scope of the invention. Rather, the scope of the invention is to be determined by the appended claims and their equivalents.

1-12. (canceled)

13. An isolated nucleic acid molecule comprising one or more of SEQ ID NOs: 1-127 and containing an alternative nucleotide at the polymorphic site that correlates with resistance to a macrocylic lactone.

14. The isolated nucleic acid molecule of claim 13, wherein the nucleic acid molecule possesses at least 80% sequence identity to one or more of SEQ ID NOs: 1-127 and contains an alternative nucleotide at the polymorphic site.

15. The isolated nucleic acid molecule of claim 13, wherein the nucleic acid molecule possesses at least 90% sequence identity to one or more of SEQ ID NOs: 1-127 and contains an alternative nucleotide at the polymorphic site.

16. The isolated nucleic acid molecule of claim 13, wherein the nucleic acid molecule possesses at least 95% sequence identity to one or more of SEQ ID NOs: 1-127 and contains an alternative nucleotide at the polymorphic site.

17-24. (canceled)

25. A method of treating an animal infected with a Dirofilaria spp. nematode, the method comprising determining the genotype of the nematode at a polymorphic site in a nucleic acid molecule that includes one or more of SEQ ID NOs: 1-127, wherein if the polymorphic site correlates with resistance to a macrocylic lactone, then the animal is treated with an alternative agent, and wherein if the polymorphic site does not correlate with resistance to a macrocylic lactone, then the animal is treated with a macrocyclic lactone.

26. The method of claim 25, wherein the nucleic acid molecule possesses at least 80% sequence identity to one or more of SEQ ID NOs: 1-127.

27. The method of claim 25, wherein the nucleic acid molecule possesses at least 90% sequence identity to one or more of SEQ ID NOs: 1-127.

28. The method of claim 25, wherein the nucleic acid molecule possesses at least 95% sequence identity to one or more of SEQ ID NOs: 1-127.

29. The method of claim 25, wherein the nucleic acid molecule includes a fragment having a length of at least 100 nucleotides of one or more of SEQ ID NOs: 1-127 and includes the polymorphic site.

30. The method of claim 25, wherein the nucleic acid molecule includes a fragment having a length of at least 50 nucleotides of one or more of SEQ ID NOs: 1-127 and includes the polymorphic site.

31. The method of claim 25, wherein the nucleic acid molecule includes a fragment having a length of at least 100 nucleotides that possesses at least 95% sequence identity to one or more of SEQ ID NOs: 1-127 and includes the polymorphic site.

32. The method of claim 25, including treating the animal with one or more alternative agents when an alternative nucleotide is found at the polymorphic site.

33. The method of claim 32, where alternative agents include one or more of an arsenic-based therapy, diethylcarbamazine, and antibiotics.

34. The method of claim 25, wherein the Dirofilaria spp. nematode is Dirofilaria immitis.

35. The method of claim 25, including isolating the nucleic acid molecule from the nematode, and optionally purifying the nucleic acids prior to determining the genotype of the nematode.

36. The method of claim 25, wherein the genotype of the nematode is determined by DNA sequencing, hybridization-based methods including with allele specific oligonucleotides, microarray analysis, enzyme-based methods, single strand conformational polymorphism (SSCP), high resolution melt (HRM) or approaches based on PCR, RT-PCR, qRT-PCR.

37. The method of claim 25, wherein the nucleic acid molecule comprises 25-300 nucleotides of SEQ ID NO: 118, or a reverse complement thereof.

38. A method of treating an animal infected with a Dirofilaria spp. nematode, the method comprising determining the genotype of the nematode at a polymorphic site in a nucleic acid molecule that includes SEQ ID NO: 118, wherein the polymorphic site is at position 151, wherein if position 151 is G, then the animal is treated with an alternative agent, and wherein if position 151 is not G, then the animal is treated with a macrocyclic lactone.

39. The method of claim 38, where alternative agents include one or more of an arsenic-based therapy, diethylcarbamazine, and antibiotics.

40. The method of claim 39, wherein the arsenic-based therapy is melarsomine dihydrochloride.
